(19) United States
(12) Patent Application Publication
(10) Pub. No.: US 2024/0287123 A1
(43) Pub. Date: Aug. 29, 2024

(54) ANTIVIRAL NUCLEOSIDE ANALOGUES

(71) Applicants: Victoria Link Limited, Wellington (NZ); Albert Einstein College of Medicine, Bronx, NY (US)

(72) Inventors: Steven C. Almo, Bronx, NY (US); Tyler Grove, Bronx, NY (US); Gary Brian Evans, Wellington (NZ); Lawrence Daniel Harris, Wellington (NZ); Rinu Shrestha, Wellington (NZ); James Michael Wood, Wellington (NZ)

(21) Appl. No.: 18/568,968

(22) PCT Filed: May 31, 2022
(86) PCT No.: PCT/NZ2022/050061

§ 371 Date: Dec. 11, 2023

Related U.S. Application Data
(60) Provisional application No. 63/209,542, filed on Jun. 11, 2021.

(51) Int. Cl. C07H 19/06; A61K 31/506; A61K 31/7068; A61P 31/18; A61P 31/22; C07D 405/04; C07H 19/10

(52) U.S. Cl. CPC C07H 19/06; A61K 31/506; A61K 31/7068; A61P 31/18; A61P 31/22; C07D 405/04; C07H 19/10

(57) ABSTRACT

Compounds of the Formula (I), pharmaceutical compositions comprising compounds of the Formula (I), and their use for treating or preventing an infection caused by Human Cytomegalovirus (HCMV), Epstein-Barr virus (EBV), or Human Immunodeficiency Virus (HIV):

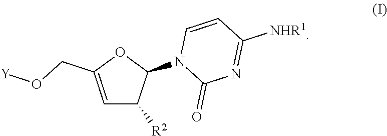

### TECHNICAL FIELD

The invention relates generally to new compounds useful for the treatment or prevention of viral infections. In particular, the invention relates to derivatives of 3′-deoxy-3′,4′-didehydro-cytidine that are active against Human Cytomegalovirus (HCMV), Epstein-Barr Virus (EBV) and Human Immunodeficiency Virus (HIV).

### BACKGROUND OF THE INVENTION

### Human Cytomegalovirus

Human Cytomegalovirus (HCMV, HHV-5), a double-stranded DNA virus of the Herpesviridae family, has a high prevalence (55-100%) within the human population.¹ Primary HCMV infection is often asymptomatic and typically results in benign latent infection. Despite the low impact of this virus in healthy hosts, neonatal infection is the leading infectious cause of congenital abnormalities in the Western world. Furthermore, primary infection or reactivation of a latent infection in immunocompromised individuals, such as organ transplant recipients or AIDS patients, can cause potentially fatal disease.

HCMV infection in immunocompromised patients is currently treated by a range of antiviral therapies and combinations of them. Five antiviral drugs are currently licensed for the treatment of HCMV infections: ganciclovir (GCV), valganciclovir (VGCV), foscarnet (FOS), cidofovir (CDV) and letermovir. The HCMV DNA polymerase, encoded for by the UL54 gene, is a common target of the antivirals foscarnet, cidofovir, ganciclovir and its prodrug valganciclovir. Foscarnet is a pyrophosphate mimic, and acts by stalling the DNA polymerase.² GCV and CDV undergo conversion to their triphosphate and diphosphate forms, respectively, which act as chain terminators downstream of their incorporation into elongating DNA.^{3,4} GCV utilises the HCMV UL97 gene-encoded kinase for the first of three phosphorylations required to generate the active triphosphate. Mutations in this gene confer resistance against GCV.⁵ CDV possesses a phosphonate group and bypasses this first phosphorylation step. However, its clinical use is limited due to its toxicity.⁶ A prodrug of CDV with reduced nephrotoxicity, brancidofovir, is currently in development, but failed to meet targets in a phase III trial.⁷ Letermovir, a viral terminase inhibitor, was approved in 2017 as a first-in-class HCMV prophylactic for bone marrow transplant recipients. It is highly selective for HCMV and is well-tolerated. However, a single mutation at UL56 C325 has been shown to confer drug resistance.⁸ Maribavir and cyclopropavir are currently in clinical trials, while other treatment strategies, such as sirtuin modulators and monoclonal antibodies, are being investigated.⁹ Despite the large number of approved small molecule drugs for HCMV, all suffer from off-target toxicity or susceptibility to drug resistance. There is thus a need for new, safer antiviral agents to prevent or treat HCMV infections.

### Epstein-Barr Virus

Epstein-Barr Virus (EBV, HHV-4) is a DNA virus of the y-herpesvirus family infecting up to 95% of the worldwide human population. For EBV to establish a successful infection it must evade or counteract the activation of type I interferons (IFN-I).¹⁰ Following primary infection, EBV establishes lifelong latency. While a functional immune system keeps latent EBV infection under control, it can cause infectious mononucleosis (glandular fever), and can lead to severe diseases such as Burkitt lymphoma (BL), diffuse large B cell lymphoma (DLBCL), and post-transplant lymphoproliferative disease (PTLD), and can be fatal in immunosuppressed and immunocompromised individuals.¹¹

Several antiviral nucleosides have been used to treat EBV associated malignancies, with limited success, but none have been approved by the FDA for use against EBV highlighting the need for the development of novel drugs for EBV.

A high level of EBV DNA in the blood of solid organ transplant recipients has shown to predict PTLD, suggesting that antiviral agents have a role in preventing PTLD. The prophylactic administration of ganciclovir, valganciclovir or acyclovir have resulted in reduced incidence of PTLD and suppression of EBV replication.^{12,13,14} Cidofovir has also been used off-label but has major limitations in terms of toxicity and pharmacokinetic liability.^{15,16} There is thus a need for new, safer anti-viral agents to prevent or treat EBV infections.

### Human Immunodeficiency Virus (HIV)

As of 2020, there were nearly 38 million people with HIV (PWH) worldwide. Over 28 million PWH have access to antiretroviral therapy (ART) drugs. These ART drugs effectively suppress HIV replication within infected cells, such as T cells, monocytes, and macrophages, but cannot eliminate the virus. Therefore, PWH take ART drugs for the rest of their lives to maintain viral suppression. The first ART drug developed in 1987 was zidovudine, a nucleoside reverse transcriptase inhibitor (NRTI). Since then, several NRTIs have been developed with greater antiviral efficacy and improved side effect profiles. Prescription of two NRTI drugs is a mainstay of both HIV treatment and pre-exposure prophylaxis (PrEP) for prevention.

Widely prescribed NRTIs, such as tenofovir and emtricitabine, can have off-target effects unrelated to viral suppression. This underscores that current NRTIs can dysregulate the vital functions of host cells by mechanisms that do not involve suppressing HIV replication. These off-target effects include liver damage, kidney toxicity, and reduced bone density. Certain NRTIs are also implicated in the development of HIV-associated comorbidities including metabolic syndromes, increased cardiovascular events, and neurocognitive impairment. Inhibition of Pol- [symbol] , which replicates mitochondrial DNA, may mediate these toxicities.

Interferons are produced early during the host response to viral infections including in HIV-infected cells. CMPK2 and RSAD2 (viperin) are major transcriptional targets that are upregulated downstream of interferon receptor signaling. These enzymes mediate the synthesis of 3′-deoxy-3′,4′-didehydro-cytidine triphosphate (ddhCTP), a chain terminating compound that inhibits several distinct viral RNA polymerases. Viral polymerases belonging to the flavivirus family and hepatitis C virus, among others, are known targets of ddhCTP. The natural intracellular abundance of ddhCTP when cells are infected with several different viruses lends itself to the development of ddhC-based antiviral therapies.

NRTI drugs based on the naturally occurring ddhCTP—that maintains potent antiviral activity with an intrinsically low toxicity at high concentrations—may overcome many issues related to the aforementioned toxicities associated with currently prescribed NRTIs and are therefore highly desirable.

### Viperin and 3′-Deoxy-3′,4′-didehydro-cytidine triphosphate (ddhCTP)

The protein viperin was first identified as an HCMV-inducible gene in fibroblasts, and was thus named virus inhibitory protein, endoplasmic reticulum (ER) associated, interferon inducible.¹⁷ This protein contains three domains: an N-terminal domain which is the least conserved region and is composed of an amphipathic a-helix and a leucine zipper domain, a highly conserved central domain and a C-terminal domain.¹⁸

The function of the central domain as a radical S-adenosyl-L-methionine (SAM) enzyme was first identified in 2018.^{19,20} Viperin is therefore a member of a super-family of enzymes that reductively cleaves SAM to generate a radical as a critical intermediate.^{20,21} Mammalian cells expressing viperin and macrophages stimulated with interferon-a produce substantial quantities of the natural antiviral ddhCTP, derived from the radically driven formal dehydration of cytidine triphosphate (CTP).^{19,20} Viperin's N-terminal amphipathic domain (consisting of a polar cytosolic-exposed side and a hydrophobic side that dips into the hydrophobic phase of cellular membranes) is postulated to contribute to the antiviral properties of viperin by intracellular localisation of viperin during viral infection. The C-terminal domain of viperin is considered critical for its antiviral function against the hepatitis C virus (HCV) and dengue virus (DENV), both members of the Flaviviridae. Likewise, the radical SAM domain contributes to the antiviral activity that viperin exhibits against HIV and Bunyamwera virus, positive and negative-sense, single-stranded enveloped RNA viruses, respectively. Viperin binds to iron sulfur clusters and reduces SAM to 5′-deoxyadenosine, a characteristic function of radical SAM enzymes.

Viperin is known to act via: (a) inhibition of viral RNA replication, (b) perturbation of the secretory pathway, (c) direct binding to viral proteins, and (d) dysregulation of lipid raft formation by altering lipid metabolism.^{18,22} In contrast, two DNA viruses, HCMV and Kaposi's sarcoma-associated herpesvirus (KSHV), have been postulated to evade the antiviral properties of viperin to their own benefit during viral replication.²²

3′-Deoxy-3′,4′-didehydro-cytidine (ddhC) is the nucleoside of the nucleotide ddhCTP, the naturally occurring metabolite produced by formal dehydration of CTP by the radical SAM enzyme viperin.^{19,20}

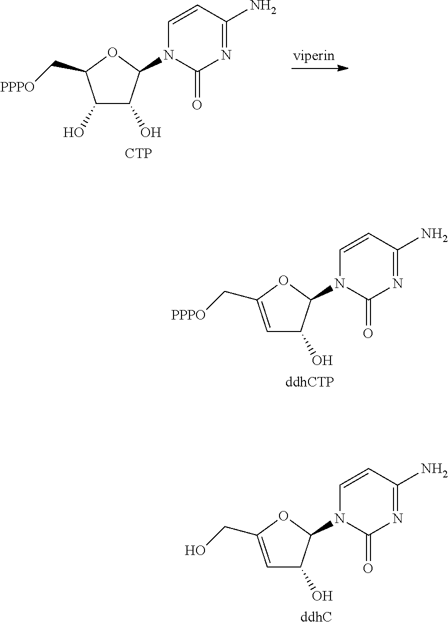

Upon its discovery, ddhCTP was found to inhibit RNA-dependent RNA polymerases (RdRps) in Flaviviridae viruses (Dengue virus, West Nile virus, Zika virus and Hepatitis C virus) by a chain termination mechanism, but at a relatively high dose, e.g. treatment of Vero cells with 1 mM ddhC was shown to reduce Zika virus proliferation. Evidently, ddhC is able to cross the cell membrane and undergo conversion to ddhCTP. Its low potency suggests it could be subject to a number of possible limitations, including innate instability,^{23,24} limited entry into cells, poor enzymatic conversion to the active antiviral ddhCTP, or poor competition of ddhCTP against CTP for incorporation by the RdRp.

While the 2′-hydroxylated ddhCTP might be considered as a candidate RdRp inhibitor, it did not seem likely that it would exhibit chain terminating activity in DNA polymerases, in having a hydroxy group where the DNA polymerase would be expected to specifically accommodate a deoxygenated methylene. Most DNA polymerases possess a steric gate to prevent ribonucleotide incorporation.²⁵

### 3′-Deoxy-3′,4′-didehydro-nucleosides

The application of certain phosphate ester derivatives of 3′-deoxy-3′,4′-didehydrogenised nucleoside compounds as antiviral medications for treating infections from HIVs, HCVs (Hepatitis C Viruses), HBVs (Hepatitis B Viruses), rabies viruses and Zika viruses has been reported.²⁶

A derivative of a compound comprising a 3′,4-didehydroribose for treating viral infections and reducing viral multiplication, including flaviviruses, has also been reported.²⁷ Reduction of viral multiplication in cells required treatment with high concentrations of ddhC.

It has been shown that a phosphoramidate derivative of ddhC undergoes more efficient conversion to ddhCTP than ddhC in Huh7 cells (a human liver cell line).²⁸ Treatment of Zika virus-infected Huh7 cells or West Nile virus-infected Huh7 cells with high concentrations of this phosphoramidate derivative resulted in reduced viral levels, though little antiviral effect was observed in Vero cells (African green monkey kidney epithelial cells) suggesting that the phosphoramidate is not efficiently converted to ddhCTP in this cell type.

The application of certain derivatives of 3′-deoxy-3′,4′-didehydro- and 3′,4′-didehydro-2′,3′-dideoxy- compounds as antiviral medications has been reported.²⁹ Some phosphate and phosphoramidate derivatives of 3′-deoxy-3′,4′-didehydro- and 3′,4′-didehydro-2′3′-dideoxy-compounds showed moderate activity against HCMV, EBV, John Cunningham virus (JCV), BK virus and Severe acute respiratory syndrome coronavirus 2 (SARS-COV-2), though cytotoxicity for these compounds was also observed.

The applicant has now surprisingly found that certain derivatives of ddhC are highly active against HCMV, moderately active against EBV and HIV, and have a high selectivity index (i.e. they are non-toxic to mammalian cells), and are therefore potential new antiviral drugs for the treatment or prevention of HCMV, EBV or HIV infections.

It is therefore an object of the invention to provide novel compounds useful for the treatment or prevention of HCMV, EBV or HIV infections, or to at least provide an alternative to known therapeutic treatments.

### SUMMARY OF THE INVENTION

In one aspect the invention provides a compound of the Formula (I):

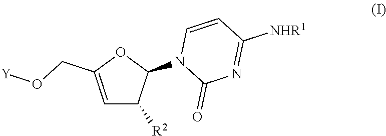

wherein:
R¹ is H, OH, a C₁₋₆ alkyl group or a cyclopropyl group;
R² is H or OH;
Y is R³, R⁴CO, PO(OR⁵)₂, PO(OR⁶)(OH), or PO(OR⁷)(X);
R³ is a pivaloyl-, isobutyroyl- or isopropyloxycarbonyloxy-(C1-3 alkyl)methyl group;
R⁴ is C₁₋₂₀ alkyl, C₆₋₁₂ aryl, C₁₋₆ alkyl-C6-aryl,

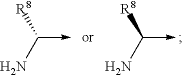

R⁵ is a S-pivaloyl-2-thioethyl, pivaloyloxymethyl or isopropyloxycarbonyloxymethyl group;
R⁶ is a 3-(C₁₂₋₁₈ alkoxy)propyl group;
R⁷ is C₆₋₁₂ aryl;
X is

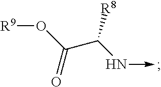

R⁸ is the side group of a natural amino acid; and
R⁹ is C₁₋₅ alkyl or C₆₋₁₂ aryl;
or a pharmaceutically acceptable salt thereof.

In some embodiments of the invention R¹ is OH, a C₁₋₆ alkyl group or a cyclopropyl group.

In some embodiments of the invention Y is R³, R⁴CO, PO(OR⁶)(OH), or PO(OR⁷)(X).

In some embodiments of the invention R⁵ is is a pivaloyloxymethyl or isopropyloxycarbonyloxymethyl group.

In some embodiments of the invention R¹ is OH, a C₁₋₆ alkyl group or a cyclopropyl group and Y is R³, R⁴CO, PO(OR⁶)(OH), or PO(OR⁷)(X).

In some embodiments of the invention R¹ is OH, a C₁₋₆ alkyl group or a cyclopropyl group and R⁵ is a pivaloyloxymethyl or isopropyloxycarbonyloxymethyl group.

In some embodiments of the invention Y is R³, RACO, PO(OR⁶)(OH), or PO(OR⁷)(X) and R⁵ is a pivaloyloxymethyl or isopropyloxycarbonyloxymethyl group.

In some embodiments of the invention R¹ is OH, a C₁₋₆ alkyl group or a cyclopropyl group, Y is R³, RACO, PO(OR⁶)(OH), or PO(OR⁷)(X), and R⁵ is a pivaloyloxymethyl or isopropyloxycarbonyloxymethyl group.

In some embodiments of the invention R¹ is H. In other embodiments R¹ is OH.

In some embodiments of the invention R² is OH.

In some embodiments of the invention R¹ is H and R² is OH.

In some embodiments of the invention Y is R³. In other embodiments Y is R⁴CO or PO(OR⁵)₂. In some embodiments of the Y contains an isopropyl group or a t-butyl group. In yet other embodiments of the invention Y is PO(OR⁵)₂, PO(OR⁶)(OH), or PO(OR⁷)(X).

Preferred compounds of the invention include, but are not limited to, the following compounds:

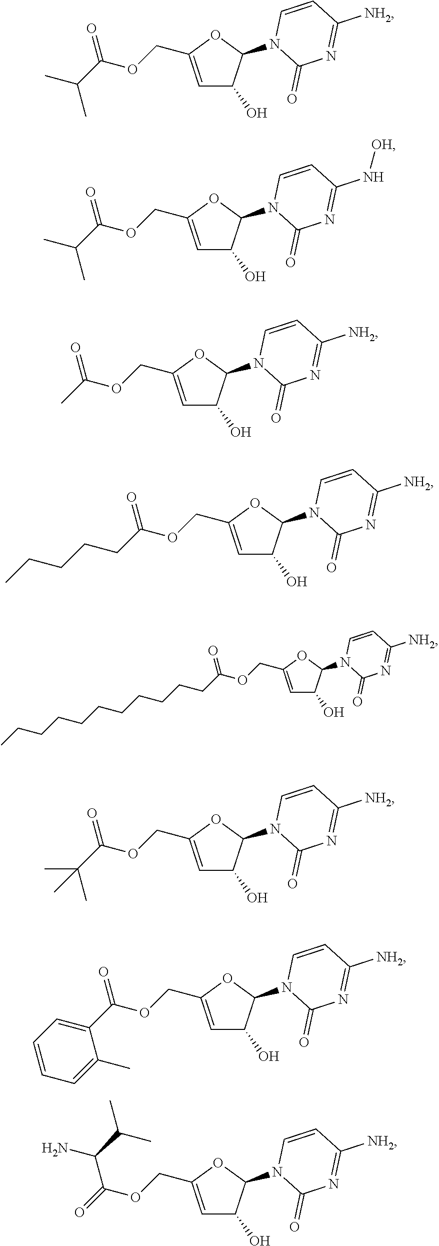

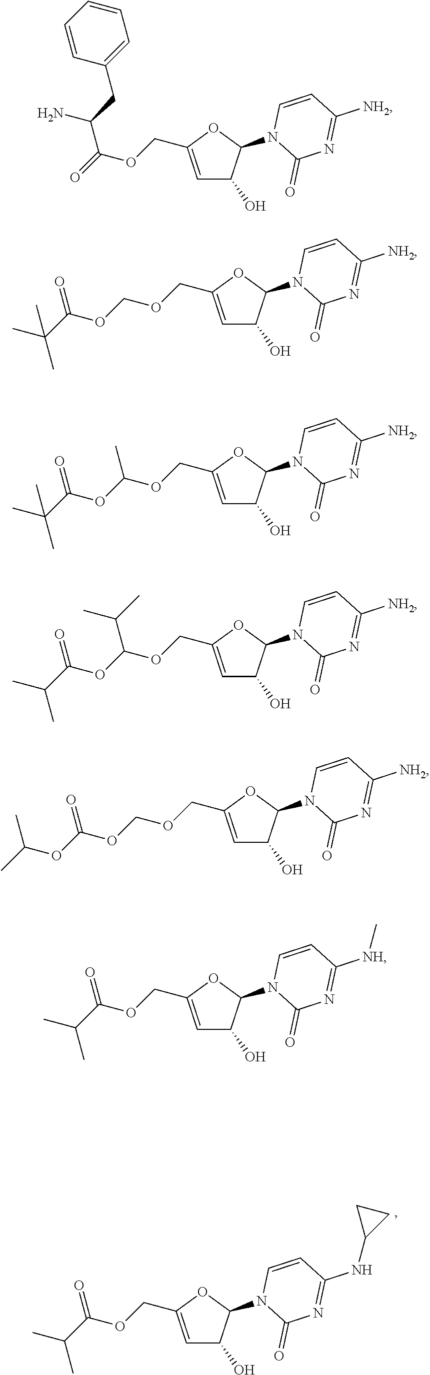

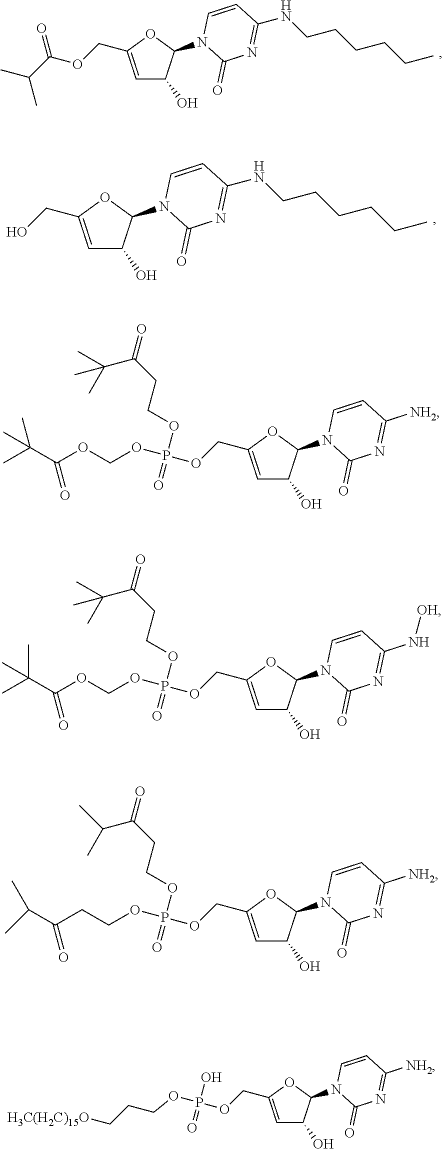

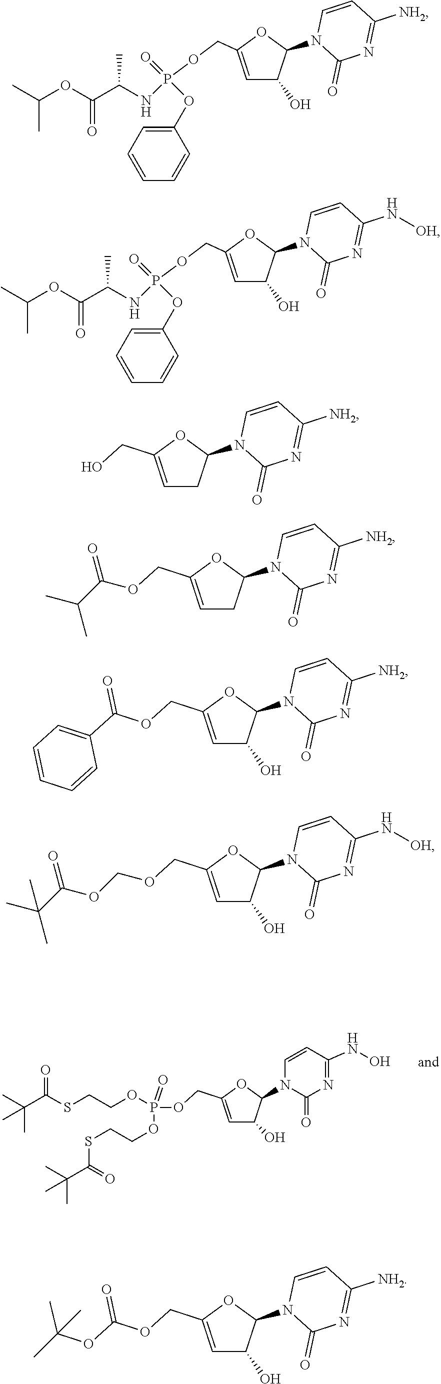

In a second aspect of the invention there is provided a pharmaceutical composition comprising a compound of the invention and a pharmaceutically acceptable carrier.

In a third aspect of the invention there is provided a method of treating or preventing an infection caused by Human Cytomegalovirus (HCMV), Epstein-Barr virus (EBV), or Human Immunodeficiency Virus (HIV) comprising administering to a human in need an effective amount of a compound of the invention.

In a further aspect of the invention there is provided the use of a compound of the invention in the manufacture of a medicament for treating or preventing an infection caused by Human Cytomegalovirus (HCMV), Epstein-Barr virus (EBV), or Human Immunodeficiency Virus (HIV).

In another aspect of the invention there is provided a composition comprising a compound of the invention for use in treating or preventing an infection caused by Human Cytomegalovirus (HCMV), Epstein-Barr virus (EBV), or Human Immunodeficiency Virus (HIV).

### BRIEF DESCRIPTION OF THE FIGURES

FIG. 1 shows the treatment of infected MDM with 100 μM ddhC.

FIG. 2 shows the treatment of infected MDM with 100 μM compound 8.

FIG. 3 shows the treatment of infected MDM with 100 μM compound 21.

FIG. 4 shows the treatment of infected MDM with DMSO control.

FIG. 5 shows the treatment of infected MDM with varying concentrations of emtricitabine and DMSO controls.

FIG. 6 is a combination of FIGS. 1-4.

FIG. 7 shows HIV p24 level fold-changes vs DMSO for 100 μM compound 8 (n=7: ** p<0.01, *** p<0.001, **** p<0.0001, one-sample t test or Wilcoxon signed rank test, relative to DMSO indicated by the dotted line at 1.0, shapes represent individual experiments).

FIG. 8 shows HIV p24 level fold-changes vs DMSO for 100 μM compound 21 (n=3: *p<0.05, ** p<0.01, one-sample t test, relative to DMSO indicated by the dotted line at 1.0, shapes represent individual experiments).

FIG. 9 shows HIV p24 levels at various doses of compound 8: (0.1-100 μM).

FIG. 10 shows HIV p24 levels at various doses of compound 21: (1-100 μM).

FIG. 11 shows HIV p24 Level fold-changes vs DMSO comparison of compounds 8 and 21 (n=3: ** p<0.01, *** p<0.001, **** p<0.0001, one-sample t test, relative to DMSO indicated by the dotted line at 1.0).

FIG. 12 shows LDH toxicity assays compared with DMSO-treated uninfected MDM.

FIG. 13 shows LDH toxicity assays compared with DMSO-treated HIV-infected MDM.

### DETAILED DESCRIPTION

### Definitions

Unless defined otherwise, all technical and scientific terms used herein have the same meaning as commonly understood to one of ordinary skill in the art to which the inventions belong. Although any assays, methods, devices and materials similar or equivalent to those described herein can be used in the practice or testing of the invention, various assays, methods, devices and materials are now described.

It is intended that reference to a range of numbers disclosed herein (for example 1 to 10) also incorporates reference to all related numbers within that range (for example, 1, 1.1, 2, 3, 3.9, 4, 5, 6, 6.5, 7, 8, 9 and 10) and also any range of rational numbers within that range (for example 2 to 8, 1.5 to 5.5 and 3.1 to 4.7) and, therefore, all sub-ranges of all ranges expressly disclosed herein are expressly disclosed. These are only examples of what is specifically intended and all possible combinations of numerical values between the lowest value and the highest value enumerated are to be considered to be expressly stated in this application in a similar manner.

As used in this specification, the words “comprises”, “comprising”, and similar words, are not to be interpreted in an exclusive or exhaustive sense. In other words, they are intended to mean “including, but not limited to”.

The term “alkyl” means any saturated hydrocarbon radical and is intended to include both straight- and branched-chain alkyl groups. The term “C₁-C₆ alkyl” means any saturated hydrocarbon radical having up to 6 carbon atoms. Examples include, but are not limited to methyl, ethyl, n-propyl, iso-propyl, n-butyl, iso-butyl, sec-butyl, t-butyl, n-pentyl, 1,1-dimethylpropyl, 1,2-dimethylpropyl, 2,2-dimethylpropyl, 1-ethylpropyl, 2-ethylpropyl, n-hexyl and 1-methyl-2-ethylpropyl.

The term “alkenyl” means any hydrocarbon radical having at least one double bond and is intended to include both straight- and branched-chain alkenyl groups. Examples of alkenyl groups include, but are not limited to, ethenyl, n-propenyl, iso-propenyl, n-butenyl, iso-butenyl, sec-butenyl, n-pentenyl, 1,1-dimethylpropenyl, 1,2-dimethylpropenyl, 1-ethylpropenyl, 2-ethylpropenyl, n-hexenyl and 1-methyl-2-ethylpropenyl.

The term “alkynyl” means any hydrocarbon radical having at least one carbon-carbon triple bond and is intended to include both straight- and branched-chain alkynyl groups. Examples of alkynyl groups include, but are not limited to, ethynyl, n-propynyl and n-butynyl.

The term “aryl” means an aromatic radical having 6 to 12 carbon atoms and includes optionally substituted aryl radicals. Examples include monocyclic groups, as well as fused groups such as bicyclic groups and tricyclic groups. Examples include, but are not limited to, phenyl, 1-naphthyl, 2-naphthyl and biphenyl. Optional substituents for aryl radicals include, but are not limited to, halide substituents such as bromine, chlorine, fluorine, and iodine; alkyl substituents such as methyl, ethyl, and propyl; and alkoxy substituents such as methoxy and ethoxy.

The term “alkoxy” means an OR group, where R is alkyl as defined above.

The term “acyl” means a —(C═O)R group, where R is alkyl, alkenyl, alkynyl, aryl as defined above.

The term “acyloxy” means an —O(C═O)R group, where R is alkyl, alkenyl, alkynyl, aryl as defined above.

The term “aryloxy” means an OR group, where R is aryl as defined above.

The term “alkylene” means a diradical corresponding to an alkyl group and is intended to include straight chain alkyl groups. Examples of alkylene groups include, but are not limited to, methylene and ethylene.

The term “protecting group” means a group that selectively protects an organic functional group, temporarily masking the chemistry of that functional group and allowing other sites in the molecule to be manipulated without affecting the functional group. Suitable protecting groups are known to those skilled in the art and are described, for example, in Protective Groups in Organic Synthesis (3^{rd} Ed.), T. W. Greene and P. G. M. Wuts, John Wiley & Sons Inc (1999). Examples of protecting groups include, but are not limited to, O-benzyl, O-benzhydryl, O-trityl, O-t-butyldimethylsilyl, O-t-butyldiphenylsilyl, O-4-methylbenzyl, O-acetyl, O-chloroacetyl, O-methoxyacetyl, O-benzoyl, 0-4-bromobenzoyl, O-4-methylbenzoyl, O-fluorenylmethoxycarbonyl and O-levulinoyl.

The term “side group of a natural amino acid” means the 2-substituent of a naturally occurring amino acid. Examples are a methyl (alanine), propan-2-yl (valine), propan-1-yl (norvaline), 2-methylpropan-1-yl(leucine), 1-methylpropan-1-yl (isoleucine), butan-1-yl (norleucine), tert-butyl (2-tert-butylglycine), phenyl (2-phenylglycine), benzyl (phenylalanine), p-hydroxybenzyl (tyrosine), indol-3-ylmethyl (tryptophan), imidazol-4-ylmethyl (histidine), hydroxymethyl (serine), 2-hydroxyethyl (homoserine), 1-hydroxyethyl (threonine), mercaptomethyl (cysteine), methylthiomethyl (S-methylcysteine), 2-mercaptoethyl (homocysteine), 2-methylthioethyl (methionine), carbamoylmethyl (asparagine), 2-carbamoylethyl (glutamine), carboxymethyl (aspartic acid), 2-carboxyethyl (glutamic acid), 4-aminobutan-1-yl (lysine), 4-amino-3-hydroxybutan1-yl (hydroxylysine), 3-aminopropan-1-yl(ornithine), 3-guanidinopropan-1-yl (arginine), 3-ureidopropan-1-yl (citrulline). Preferred-amino acid side groups are methyl (alanine), propan-2-yl (valine), 2-methylpropan-1-yl (leucine), benzyl (phenylalanine), imidazol-4-ylmethyl (histidine), hydroxymethyl (serine), 1-hydroxyethyl (threonine), 4-aminobutan-1-yl(lysine), 3-aminopropan-1-yl (ornithine), 2-aminoethyl (2,4-diaminobutyric acid), aminomethyl (2,3-diaminopropionic acid), 3-guanidinopropan-1-yl (arginine).

The term “silyl ether protecting group” is a protecting group such as O-t-butyldimethylsilyl and O-t-butyldiphenylsilyl.

The term “pharmaceutical composition” means a mixture of one or more of the compounds of the invention, or pharmaceutically acceptable salts, or hydrates thereof, with other chemical components, such as physiologically acceptable carriers and excipients. The purpose of a pharmaceutical composition is to facilitate administration of a compound to an organism.

The term “carrier” refers to a diluent, adjuvant, excipient, or vehicle with which a compound is administered. Non-limiting examples of such pharmaceutical carriers include liquids, such as water and oils, including those of petroleum, animal, vegetable or synthetic origin, such as peanut oil, soybean oil, mineral oil, sesame oil and the like. The pharmaceutical carrier may also be saline, gum acacia, gelatin, starch paste, talc, keratin, colloidal silica, urea, and the like. In addition, auxiliary, stabilizing, thickening, lubricating, and colouring agents may be used. Other examples of suitable pharmaceutical carriers are described in “Remington's Pharmaceutical Sciences” by E. W. Martin, herein incorporated by reference.

The term “pharmaceutically acceptable salt” refers to any salt of a compound provided herein which retains its biological properties and which is not toxic or otherwise undesirable for pharmaceutical use and is intended to include salts derived from inorganic or organic acids including, for example hydrochloric, hydrobromic, sulfuric, nitric, perchloric, phosphoric, formic, acetic, lactic, maleic, fumaric, succinic, tartaric, glycolic, salicylic, citric, methanesulfonic, benzenesulfonic, benzoic, malonic, trifluoroacetic, trichloroacetic, naphthalene-2 sulfonic and other acids. Pharmaceutically acceptable salt forms may also include forms wherein the ratio of molecules comprising the salt is not 1:1. For example, the salt may comprise more than one inorganic or organic acid molecule per molecule of base, such as two hydrochloric acid molecules per molecule of compound of formula (I).

The terms “treatment”, “treating” and the like include the alleviation of one or more symptoms, or improvement of a state associated with the disease or disorder.

The terms “preventing”, “prevention” and the like include the prevention of one or more symptoms associated with the disease or disorder.

### Synthesis of Compounds

The compounds of the invention may be prepared by any suitable method. One suitable method involves modifying a commercially available nucleoside or nucleoside derivative.

For example, Scheme 1 below outlines the synthesis of 5′-O-acyl esters of pyrimidine 3′-deoxy-3′,4′-didehydronucleosides from the commercially available N-benzoyl-cytidine (A-1) via intermediate A-6. Regioselective silylation of the 2′- and 5′-hydroxy groups enables 3′-selective Appel reaction on A-2, to generate an iodide A-3 in the D-xylo-configuration. Regioselective 5′-O-silyl deprotection to A-4 can be achieved with TFA-H₂O in THF, which on treatment with a tertiary alkylamine base, such as DABCO, enables elimination of HI to give a 3′,4′-didehydro-compound A-5. The N-4 protecting group cleavage can be performed using ammonia in methanol to deliver A-6.

Introduction of a 5′-O-acyl ester to give A-7 can be achieved by treatment of A-6 with an acid anhydride, tertiary amine base and DMAP catalyst. Alternatively, if the acid anhydride is not readily available, the acyl ester can be introduced by treatment with the appropriate carboxylic acid and a coupling agent such as EDC, or under Mitsunobu conditions using a diazodicarboxylate and triphenylphosphine.

5′-O-(Amino acid) esters of pyrimidine 3′-deoxy-3′,4′-didehydronucleosides can be prepared by this latter method, using an Fmoc-protecting strategy for the α-amino group. This fluorenylmethyl carbamate can be cleaved after esterification using an alkylamine base.

After acylation to give A-7, substituted 4-amino group can be introduced on the cytidine moiety by transamination to give A-9. Thus, introduction of hydroxylamine at this position can be achieved by treatment of A-7 with hydroxylamine and mild acid.³⁰ Alternatively, an alkylamine can be introduced by treatment with the appropriate alkylamine and bisulfite,³¹ or alternatively by reductive amination of A-7 with the appropriate aldehyde or dialkylacetal.^{32,33} Cleavage of the 2′-O-silyl ether can be achieved using a fluoride source such as 3HF·Et₃N to yield compounds of the invention A-8 or A-10.

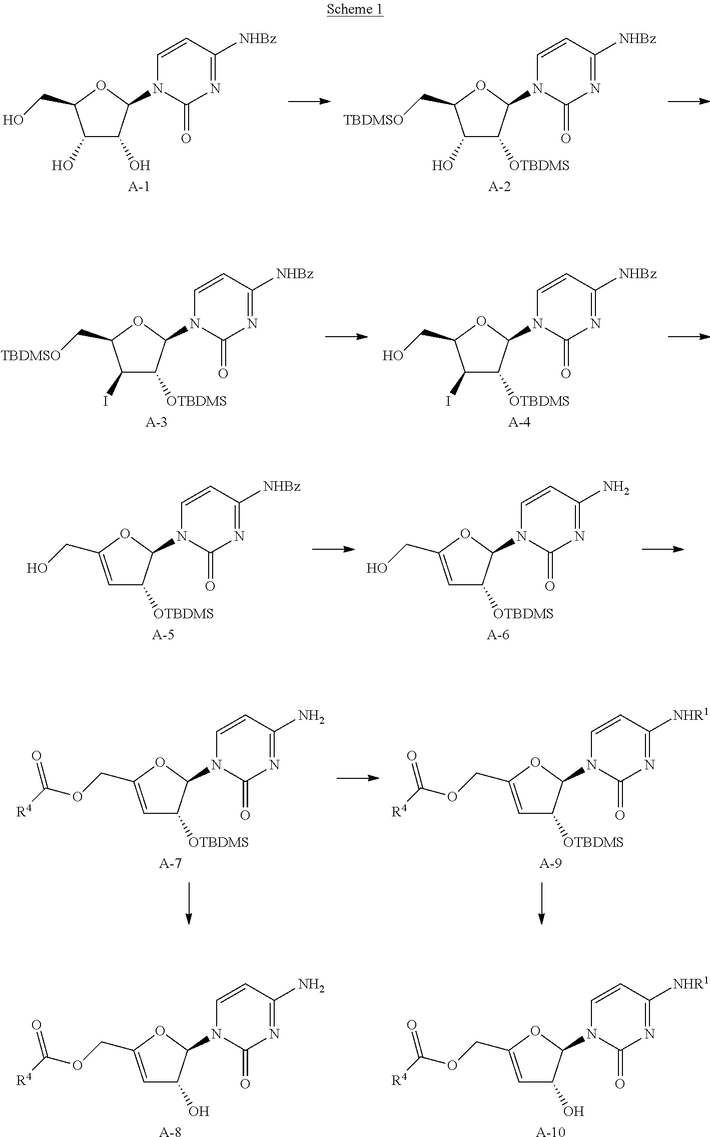

Pyrimidine 3′-deoxy-3′,4′-didehydronucleosides with substitution at N-4 can also be prepared from uridine (A-11) as shown in Scheme 2. Regioselective silylation of the 2′- and 5′-hydroxy groups enables 3′-selective Appel reaction, then elimination of HI to generate 3′-deoxy-3′,4′-didehydrouridine (A-14). Activation of the uracil ring at C-4, then treatment with the appropriate amine nucleophile provides the 4-N-modified cytidine derivative A-15. Methods of activation include sulfonylation with a hindered arylsulfonyl chloride, such as 2,4,6-triisopropylbenzenesulfonyl chloride, or generation of a C4-triazol-1-yl intermediate by treatment with phosphoryl chloride and 1,2,4-triazole. Global deprotection to yield A-16 can be achieved with an appropriate fluoride source such as 3HF·Et₃N.

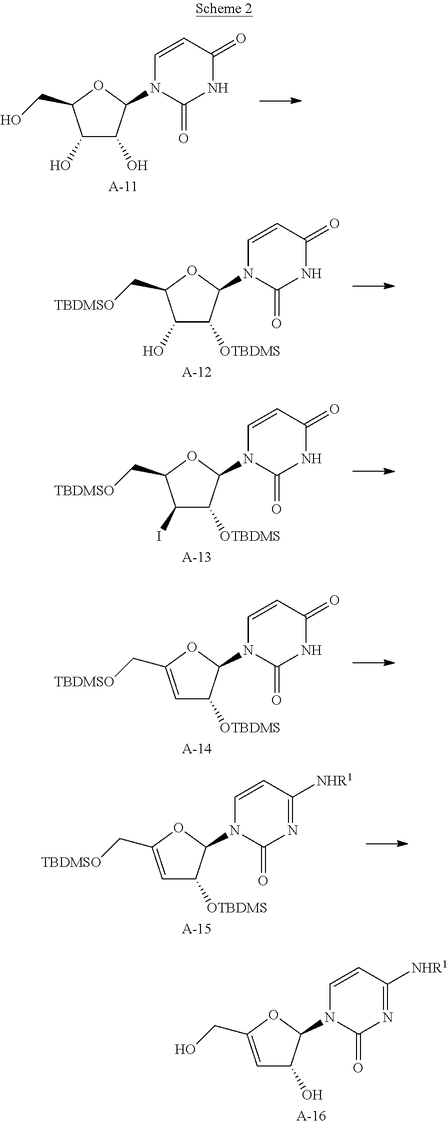

Phosphate and phosphoramidate esters of ddhC, can be prepared from intermediate A-6 by nucleophilic substitution of an appropriate phosphorochloridate, or phosphoramidochloridate, respectively, in the presence of base (Scheme 3). Alternatively, phosphorus(V) reagents bearing a different leaving group, such as pentafluorophenol, can be employed. Phosphate esters of ddhC may also be prepared from intermediate A-6 by treatment with an appropriate phosphoramidite and 1H-tetrazole, followed by oxidation of the resultant phosphite with t-BuOOH. Phosphate monoesters of ddhC (A-19) can be prepared from intermediate A-6 by treatment with an appropriate O-cyanoethyl phosphoramidite and 1H-tetrazole, followed by oxidation of the resultant phosphite with t-BuOOH, and elimination of the cyanoethyl group with base. Following introduction of the phosphorus moiety, the 2′-O-silyl ether can be cleaved by either a fluoride source or aqueous acetic acid to yield the desired 5′-O-phosphorus esters A-17, A-18 or A-19, respectively.

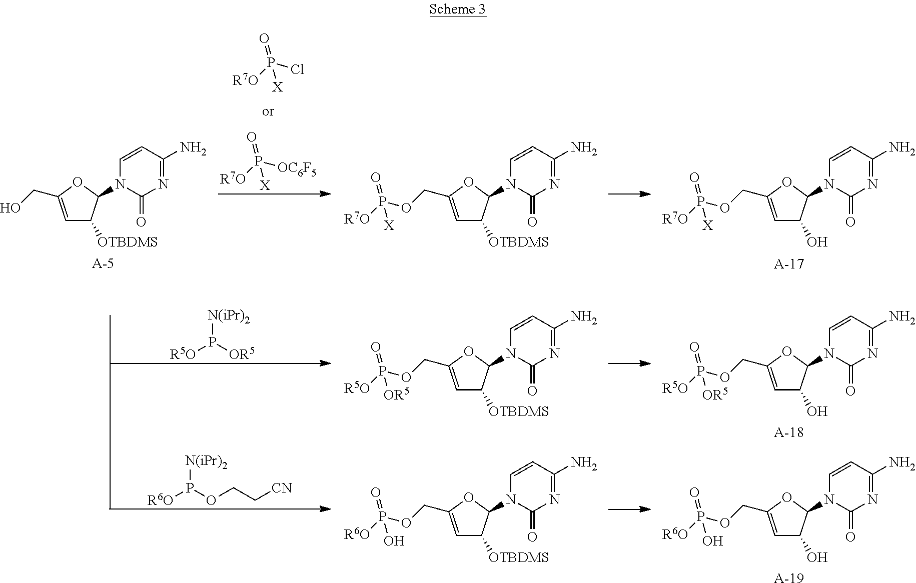

Phosphate esters of pyrimidine 3′-deoxy-3′,4′-didehydronucleosides with substitution at N-4 can also be prepared from 3′-deoxy-3′-iodouridine intermediate A-13 as shown in Scheme 4. Regioselective 5′-O-silyl deprotection to A-20 can be achieved with TFA-H₂O in THF, which on treatment with a tertiary alkylamine base, such as DABCO, enables elimination of HI to give a 3′,4′-didehydro-compound A-21. Treatment with an appropriate phosphoramidite and 1H-tetrazole, followed by oxidation of the resultant phosphite with t-BuOOH affords a phosphate ester A-22. Activation of the uracil ring at C-4 by sulfonylation with a hindered arylsulfonyl chloride, then treatment with the appropriate amine nucleophile provides the 4-N-modified cytidine phosphate ester derivative A-23. Silyl deprotection to yield A-24 can be achieved with an appropriate fluoride source such as 3HF·Et₃N. Similarly, the phosphoramidate esters or phosphate monoesters of pyrimidine 3′-deoxy-3′,4′-didehydronucleosides with substitution at N-4 can be prepared from compound A-21 following the general methodologies outlined in Scheme 4.

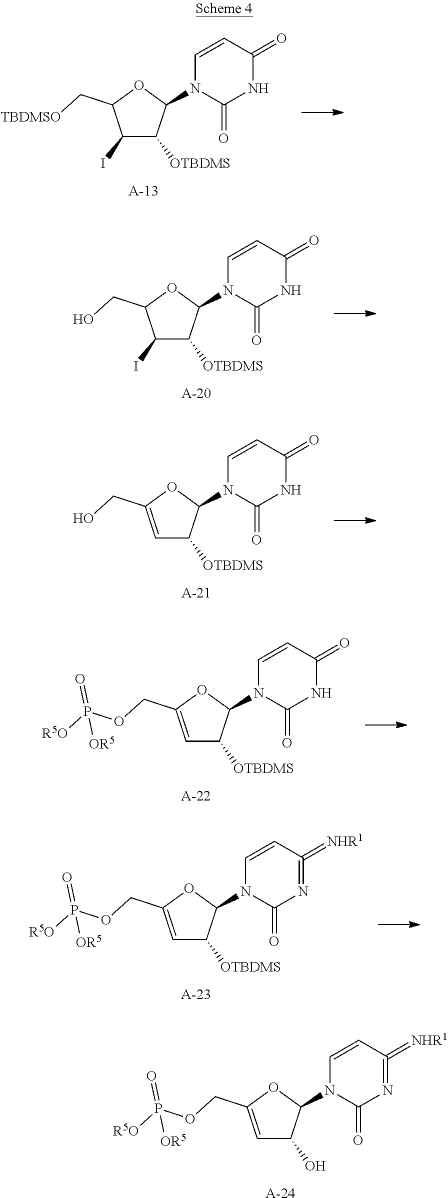

3′,4′-Didehydro-2′,3′-dideoxycytidine (A-30) and it's 5-O-acyl esters can be prepared from commercially available 2′-deoxycytidine (A-25) as shown in Scheme 5. Regioselective silylation of the 5′-hydroxy group, followed by benzoylation affords benzoate A-27. 5′-O-silyl deprotection to A-28 can be achieved using TFA-H₂O, which on Moffatt-Pfitzner oxidation to a 5′-aldehyde and treatment with Et₃N, enables benzoic acid elimination to afford 3′,4′-didehydro aldehyde A-29. Reduction of aldehyde A-29 with NaBH4 and Zemplén 4-N-benzoyl cleavage affords 3′,4′-didehydro-2′,3′-dideoxycytidine (A-30). Introduction of a 5′-O-acyl ester to give A-31 can be achieved by treatment of alcohol A-30 with an acid anhydride, tertiary amine base and DMAP catalyst. Similarly, phosphate diesters, phosphoramidate esters or phosphate monoesters of 3′,4′-didehydro-2′,3′-dideoxycytidine (A-30) can be prepared directly from A-30 following the general methodologies outlined in Scheme 5.

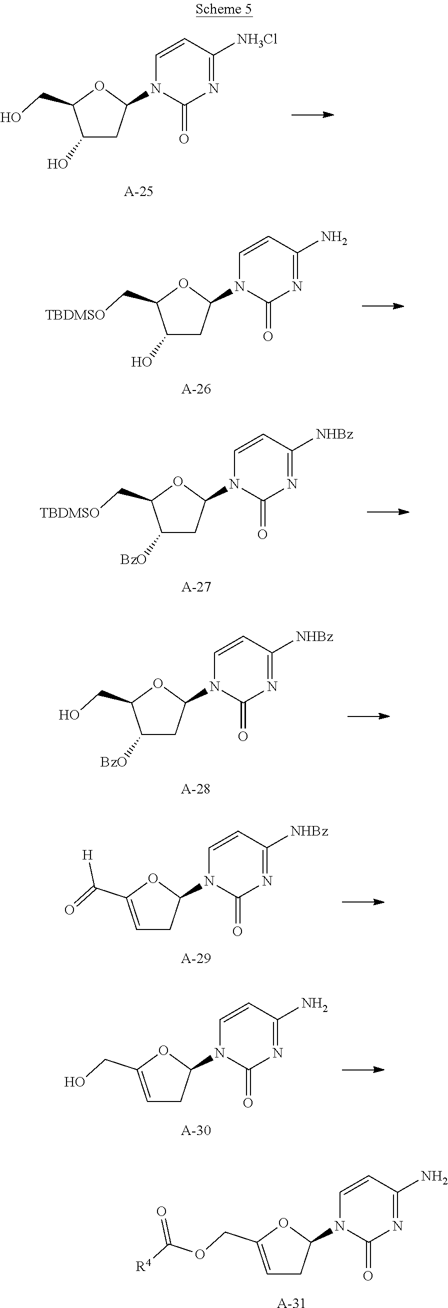

### Antiviral Activity

The antiviral activities of ddhC (prepared according to the method of Petrová)³⁴ and compounds 8, 10 and 21 were assessed against HCMV, EBV and HIV. See Example 16.

Compounds 8 and 10 displayed strong activity against HCMV, with EC₅₀ values of approximately 20 nM and 30 nM, respectively. All of the compounds (ddhC, 8, 10 and 21) displayed moderate activity with ECso values between 7.6-38.7 μM against EBV. Compound 8 was the most active compound at 7.6 μM and is more potent than the control cidofovir (CDV) which had an EC₅₀ of 8.25 μM.

The promiscuous HCMV UL97 kinase is a known potent activator of ganciclovir to generate ganciclovir monophosphate, and likely efficiently processes the ddhC formed in cells to the monophosphate. Without being bound by theory, the applicants consider that the monophosphate is probably converted by cellular kinases to the triphosphate which likely acts as a DNA chain terminator when acted upon by the viral DNA polymerase thereby effecting the observed antiviral activity.

EBV encodes a thymidine kinase (EBV-TK), and a protein kinase (EBV-PK) which is a homologue of the protein encoded by the UL97 gene of HCMV. Without wishing to be bound by theory, EBV-PK likely phosphorylates the ddhC formed in cells, following esterase-mediated cleavage of the isobutyroyl-group of 8 or 10. Surprisingly, ddhC does not display antiviral activity against HCMV and is five-fold less active than compound 8 against EBV, suggesting that the more lipophilic compound 8 accumulates in cells far more rapidly than ddhC. This likely leads to concentrations of ddhCTP necessary for effective DNA chain termination when acted upon by the viral DNA polymerases of HCMV and EBV. Compound 10 probably exerts its antiviral activity by a similar mechanism. Moreover, these compounds displayed no toxicity to the cells in which they were tested, at a concentration of 50 μM.

Compound 8 performed most favourably compared with the cidofovir control to inhibit EBV replication. Other 5′-acyl-ddhC derivatives 23, 25 and 30 were also comparably active, apart from compound 27 which was inactive (at a concentration of below 50 μM in this this cell-based assay—the highest concentration tested in primary assays for EBV).

ddhC only weakly inhibited EBV. This combined with the data showing inhibition for more lipophilic compounds, for example compounds 8, 23 and 25, suggests that ddhC does not effectively enter cells.

The inclusion of the N-hydroxy moiety in compound 10 slightly diminishes the activity compared with compound 8. Unlike the 5′-acyl-ddhC derivative 23, the phosphoramidate derivative 21 is more weakly inhibiting. In contrast, phosphoramidate derivative 32 is not active (at a concentration of below 50 μM—the highest concentration tested in primary assays for EBV). Similarly, the pivaloyloxymethyl-derivatised phosphate ester 35 is also inactive (at a concentration of below 50 μM—the highest concentration tested in primary assays for EBV). The 2′-deoxy-ddhC derivative A-30 and its isobutyroyl derivative 39 both displayed activity comparable to their 2′-hydroxylated analogues (ddhC and 8).

The applicant has demonstrated the anti-HIV properties of compounds 8 and 21 in primary human macrophages, which are significant tissue targets for HIV. Monocyte derived macrophages (MDMs) are natural targets for HIV infection and can serve as long-lived reservoirs for HIV that exist in tissues including lymph nodes and the brain.

Primary human MDMs were cultured from peripheral blood mononuclear cells and pre-treated for 24 h with ddhC (100 μM), compound 8 (100 μM), compound 21 (100 μM), emtricitabine control (15 μM, 10 μM, 1 μM, 0.1 μM), or the drug vehicle, DMSO. MDM were then infected with HIV_{ADA} in the presence of these compounds and treated daily (with compound), while culture supernatants were collected to quantify levels of HIV p24, a viral capsid protein, on days 2-7 of infection.

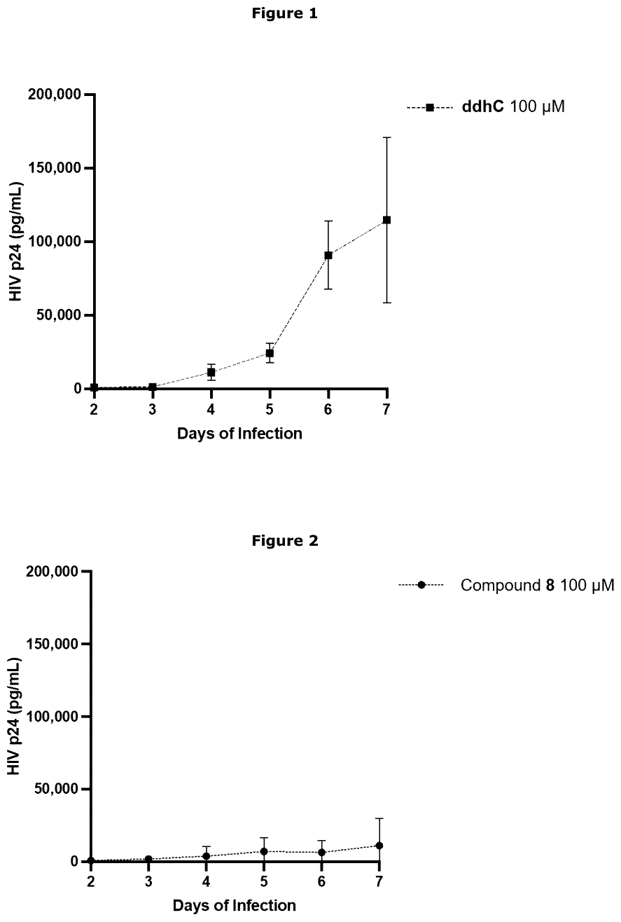

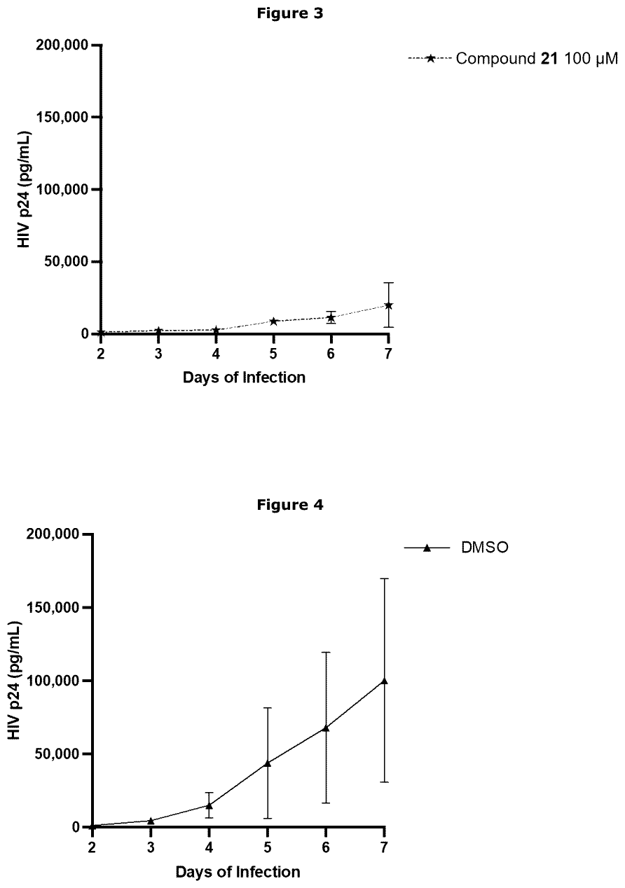

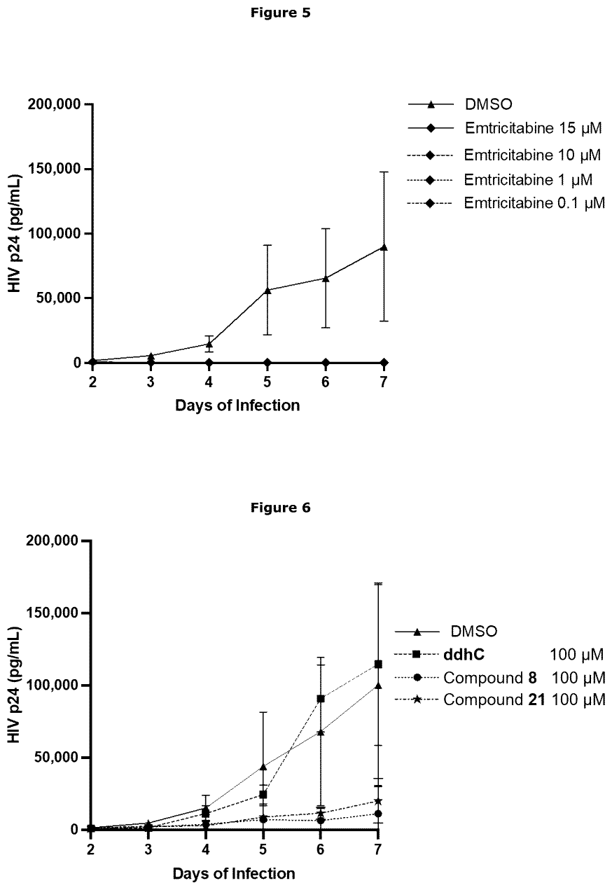

Compounds 8 and 21 both significantly reduce the levels of p24 viral capsid protein (FIGS. 2 and 3, respectively), compared with ddhC (FIG. 1) or DMSO control (FIG. 4), as the infection progresses. The observed reductions in the levels of p24 viral capsid protein when MDMs are treated with compounds 8 or 21 are similar in effect to what is observed with the commonly prescribed nucleoside reverse transcriptase inhibitor (NRTI) emtricitabine (FIG. 5). FIG. 6 is an overlay of FIGS. 1-4 showing the comparison of the reduction of p24 levels by ddhC (100 μM), compound 8 (100 μM), compound 21 (100 μM), or the drug vehicle, DMSO.

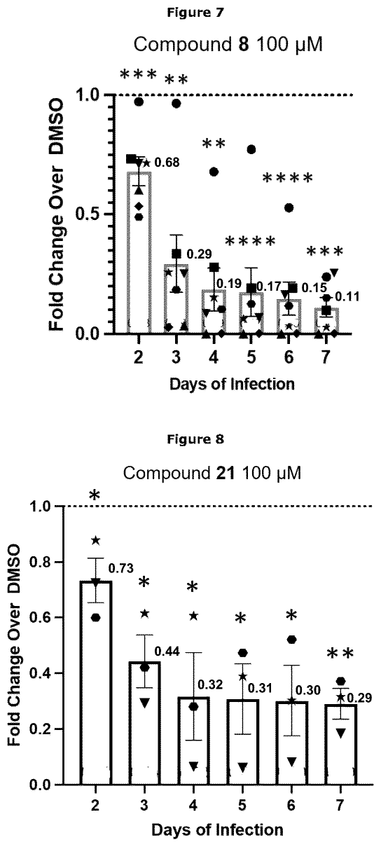

Due to variability in baseline levels of HIV infection in primary human samples, raw p24 values (FIGS. 1-5) were converted to normalized fold-change values (set to 1.0 in each experiment) relative to DMSO controls. Treatment with either compound 8 (100 μM) (FIG. 7) or compound 21 (100 μM) (FIG. 8) significantly decreased levels of HIV p24, while no reduction was observed for ddhC compared with DMSO control. This indicated potent inhibition of viral replication by either compound 8 or compound 21.

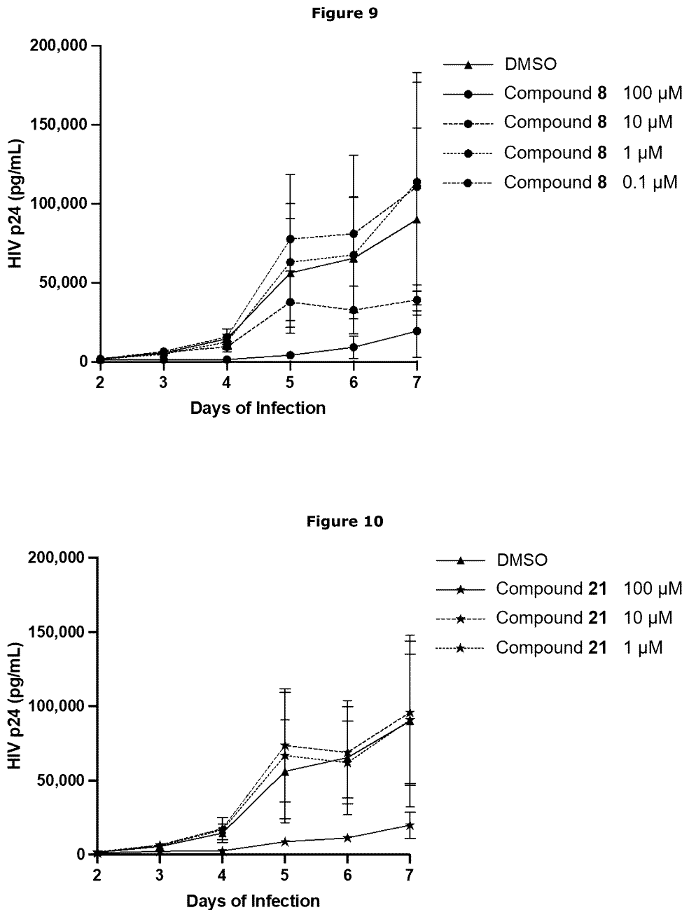

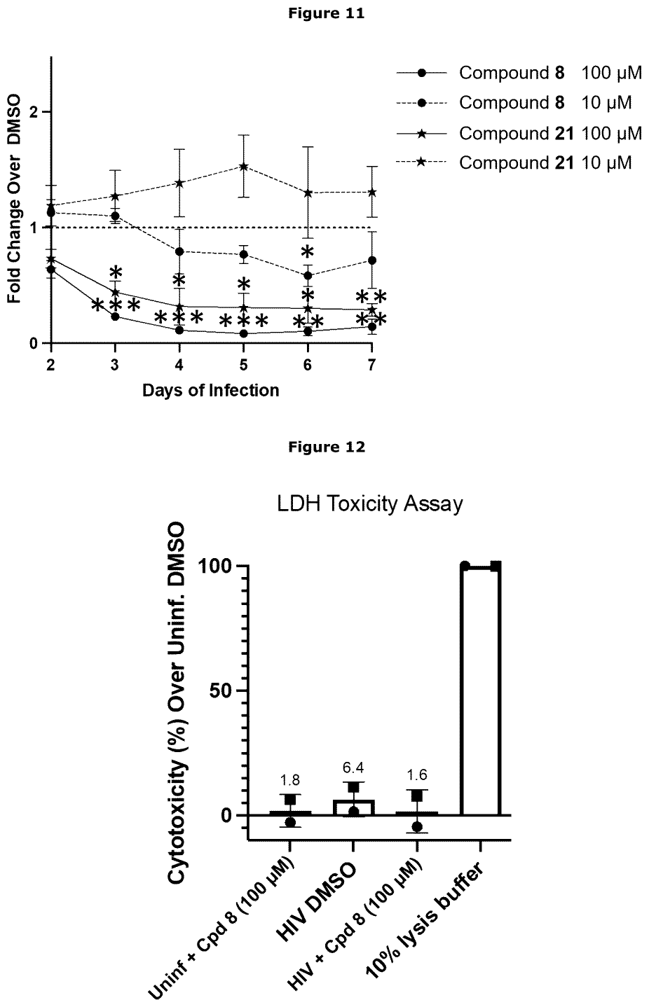

The impact of various doses of compound 8 and compound 21 on HIV p24 levels at days 2-7 of infection was also examined (FIGS. 9-11). Compound 8 (100 μM) and compound 21 (100 μM) significantly reduced p24 levels as described earlier, and compound 8 (10 μM) reduced p24 levels ˜50% by day 6 of infection, which was statistically significant (FIG. 11). Compound 8 (0.1-1 M) and 1-10 μM compound 21 (0.1-1 μM) both had minimal impact on HIV replication relative to the DMSO control.

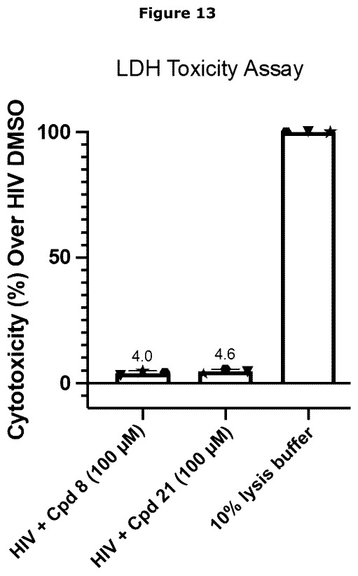

Both compound 8 and compound 21 were determined to be non-toxic to MDMs at 100 uM by both visual inspection by light microscopy and colorimetric lactate dehydrogenase (LDH) assays. Light microscopy was used to image MDMs on day 7 of infection. Highly infected MDM fuse together to form syncytia which were evident in the DMSO treated control. Conversely, cells treated at 100 μM of compound 8 or compound 21 looked like uninfected MDM with normal size and confluency. Colorimetric LDH assays were also performed on culture supernatants from these same cells on day 4 of infection to assess cell death. There was no significant difference in LDH activity in uninfected or HIV-infected MDMs that had been treated with 100 μM compound 8 (100 μM), relative to uninfected DMSO controls (FIG. 12). LDH activity was also nearly identical between HIV-infected MDMs treated with 100 μM of compounds 8 and 21 MDM relative to DMSO controls (FIG. 13). Thus, daily treatment of primary human MDM with these drugs was non-toxic.

### Formulations and Administration

The compounds of the invention may be administered to a patient by a variety of routes, including orally, parenterally, by inhalation spray, topically, rectally, nasally, buccally or via an implanted reservoir. For parenteral administration, injections may be given intravenously, intra-arterially, intramuscularly or subcutaneously.

The amount of a compound of the invention to be administered to a patient will vary widely according to the nature of the patient and the nature and extent of the disorder to be treated. Typically, the dosage for an adult human will be in the range of about 0.01 μg/kg to about 1 g/kg, and preferably about 0.01 mg/kg to about 100 mg/kg. The specific dosage required for any particular patient will depend upon a variety of factors, such as the patient's age, body weight, general health, gender and diet. Optimal doses will depend on other factors such as mode of administration and level of progression of the disease or disorder. Doses may be given once daily, or two or more doses may be required per day. For example, a dosage regime for a malaria patient might require one dose in the morning and one in the evening. Alternatively, a dosage regime for such a patient might require four hourly doses.

For oral administration the compounds can be formulated into solid or liquid preparations, for example tablets, capsules, granules, powders, solutions, suspensions, syrups, elixirs and dispersions. Such preparations are well known in the art as are other oral dosage regimes not listed here.

For parenteral administration, compounds of the invention can be formulated into sterile solutions, emulsions and suspensions.

Compounds of the invention may be mixed with suitable vehicle and then compressed into the desired shape and size. The compounds may be tableted with conventional tablet bases such as lactose, sucrose and corn starch, together with a binder, a disintegration agent and a lubricant. The binder may be, for example, corn starch or gelatin, the disintegrating agent may be potato starch or alginic acid, and the lubricant may be magnesium stearate. For oral administration in the form of capsules, diluents such as lactose and dried cornstarch may be employed. Other components such as colourings, sweeteners or flavourings may be added. Tablets, capsules or powders for oral administration may contain up to about 99% of a compound of the invention.

When liquid preparations are required for oral use, a compound of the invention may be combined with a pharmaceutically acceptable carriers such as water, an organic solvent such as ethanol, or a mixture of both, and optionally other additives such as emulsifying agents, suspending agents, buffers, preservatives, and/or surfactants may be used. Colourings, sweeteners or flavourings may also be added.

The compounds may also be administered by injection in a pharmaceutically acceptable diluent such as water or saline. The diluent may comprise one or more other ingredients such as ethanol, propylene glycol, an oil or a pharmaceutically acceptable surfactant.

The compounds of the invention may also be administered topically. Carriers for topical administration of the compounds include mineral oil, liquid petrolatum, white petrolatum, propylene glycol, polyoxyethylene, polyoxypropylene compound, emulsifying wax and water. The compounds may be present as ingredients in lotions or creams, for topical administration to skin or mucous membranes. Such creams may contain the active compounds suspended or dissolved in one or more pharmaceutically acceptable carriers. Suitable carriers include mineral oil, sorbitan monostearate, polysorbate 60, cetyl ester wax, cetearyl alcohol, 2-octyldodecanol, benzyl alcohol, and water.

The compounds of the invention may further be administered by means of sustained release systems. For example, they may be incorporated into a slowly dissolving tablet or capsule.

Any reference to prior art documents in this specification is not to be considered an admission that such prior art is widely known or forms part of the common general knowledge in the field.

The invention is further described with reference to the following Examples. It will be appreciated that the invention as claimed is not intended to be limited in any way by these Examples.

### EXAMPLES

### Example 1: Preparation of 2′-O-(tert-Butyldimethylsilyl)-3′-deoxy-3′,4′-didehydrocytidine (6)

### Example 1.1 4-N-Benzoyl-2′,5′-bis-O-(tert-butyldimethylsilyl) cytidine (2)

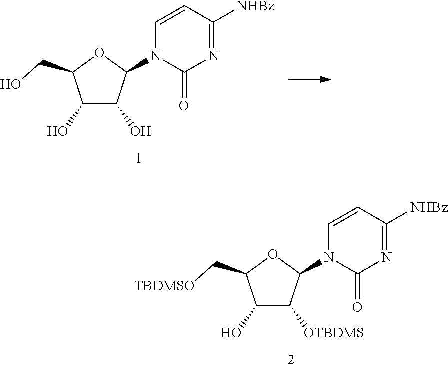

The title compound 2 was prepared according to a literature procedure.³⁵ To a suspension of 4-N-benzoylcytidine (1) (10.0 g, 28.8 mmol) in anhydrous pyridine (58 mL) at room temperature was added tert-butyldimethylsilyl chloride (13.4 g, 86.2 mmol). The reaction mixture was stirred for 3 d, then concentrated in vacuo. The resultant oil was dissolved in CHCl₃ (150 mL) and washed with 0.5 M aq. HCl (50 mL). The aqueous layer was back-extracted with CHCl₃ (50 mL), then the combined organic layers were washed with water (40 mL), brine (40 mL), dried over anhydrous MgSO₄, filtered and concentrated in vacuo. Purification by flash column chromatography (9×15 cm silica gel, 20%-60% EtOAc-Hex) afforded the title compound 2 (11.5 g, 69% yield) as a colourless foam. ¹H NMR (500 MHZ, CDCl₃) δ 8.67-8.57 (m, 1H), 8.53 (d, J=4.4 Hz, 1H), 7.90 (d, J=5.4 Hz, 2H), 7.63-7.59 (m, 1H), 7.54-7.49 (m, 2H), 5.98 (d, J=2.2 Hz, 1H), 4.24 (dd, J=4.6, 2.2 Hz, 1H), 4.19-4.05 (m, 3H), 3.91-3.87 (m, 1H), 2.45 (d, J=8.4 Hz, 1H), 0.97 (s, 9H), 0.94 (s, 9H), 0.28 (s, 3H), 0.17 (s, 3H), 0.16 (s, 3H), 0.16 (s, 3H).

Example 1.2 1-(2′,5′-Bis-O-(tert-butyldimethylsilyl)-3′-iodo-ß-D-threo-pentofuranosyl)-4-N-benzoylcytosine (3)

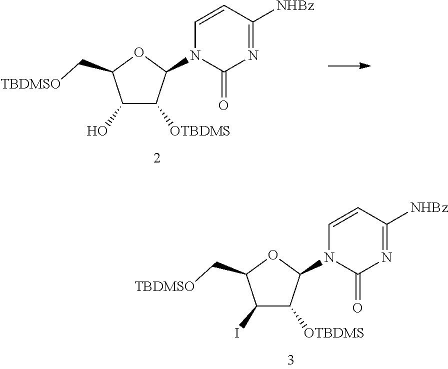

A flask was charged with methyltriphenoxyphosphonium iodide (6.41 g, 11.3 mmol) under argon, followed by alcohol 2 (4.35 g, 7.55 mmol). The solids were dissolved in DMF (50 mL), then pyridine (1.25 mL, 15.4 mmol) was added and the reaction mixture was stirred at room temperature for 18 h. The reaction mixture was quenched by addition of Et₃N (5 mL) and MeOH (5 mL), then stirred for 10 min. The reaction mixture was partitioned between water (500 mL) and EtOAc (50 mL), then the aqueous layer was extracted with EtOAc (2×50 mL). The combined organic layers were washed with brine (25 mL), dried over anhydrous MgSO₄, filtered and concentrated in vacuo. The crude oil was purified by flash column chromatography (silica gel, 10-30% EtOAc-Hex) to afford the title compound 3 (3.38 g, 65%) as an off-white solid. ¹H NMR (500 MHz, CDCl₃) δ 8.23 (d, J=7.5 Hz, 1H), 7.89 (d, J=7.8 Hz, 2H), 7.60 (t, J=7.4 Hz, 1H), 7.50 (t, J=7.7 Hz, 3H), 5.75 (d, J=1.7 Hz, 1H), 4.79 (t, J=1.8 Hz, 1H), 4.13 (dd, J=4.1, 1.8 Hz, 1H), 4.07-3.99 (m, 2H), 3.82 (dd, J=10.3, 5.1 Hz, 1H), 0.94 (s, 9H), 0.92 (s, 9H), 0.20 (s, 3H), 0.16 (s, 6H), 0.14 (s, 3H); 13C NMR (126 MHz, CDCl₃) δ 166.3, 162.4, 154.6, 145.3, 133.3, 129.2, 127.7, 95.7, 93.7, 83.8, 81.9, 68.0, 30.5, 26.0, 25.8, 18.5, 18.0, −4.6, −4.8, −5.0, −5.1; HRMS (ESI+): Calculated for C₂₈H₄₅N₃O₅Si₂I: 686.1937. Found [M+H]⁺: 686.1950.

### Example 1.3 1-(2′-O-(tert-Butyldimethylsilyl)-3′-iodo-B-D-threo-pentofuranosyl)-4-N-benzoylcytosine (4)

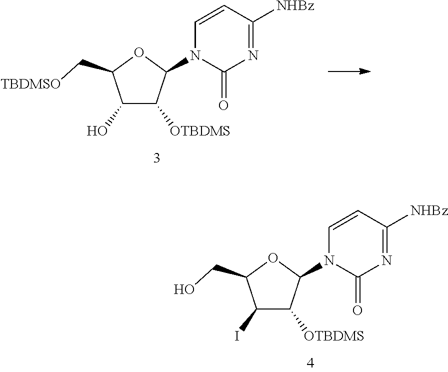

To a solution of silyl ether 3 (5.64 g, 8.23 mmol) in THF (24 mL) at δ ° C.was added a mixture of TFA-H₂O (1:1, 7.2 mL) dropwise over 3 min. The reaction mixture was warmed to room temperature and stirred for 3.5 h. The reaction was neutralized by addition of sat. aq NaHCO₃ (50 mL), then extracted with EtOAc (3×50 mL). The combined organic layers were washed with sat. aq. NaHCO₃ (20 mL), brine (20 mL), dried over anhydrous MgSO₄, filtered and concentrated in vacuo. The crude oil was purified by flash chromatography (silica gel, 10%-100% EtOAc-Hex) to afford the title compound 4 (4.17 g, 89%) as a colourless solid. [α]_{D}²⁰+13.7 (0.088, CHCl₃); ¹H NMR (500 MHZ, CDCl₃) δ 8.92 (s, 1H), 8.20 (d, J=7.6 Hz, 1H), 7.89 (d, J=8.7 Hz, 2H), 7.62-7.47 (m, 4H), 5.67 (d, J=1.5 Hz, 1H), 4.88 (t, J=1.7 Hz, 1H), 4.18-4.04 (m, 3H), 3.88 (dd, J=11.7, 4.3 Hz, 1H), 0.91 (s, 9H), 0.20 (s, 3H), 0.16 (s, 3H); ¹³C NMR (126 MHZ, CDCl₃) δ 166.6, 162.7, 155.1, 145.7, 133.3, 129.2, 127.7, 96.0, 94.6, 83.7, 82.0, 68.0, 29.3, 25.8, 18.0, −4.6, −4.8; HRMS (ESI+): Calculated for C₂₂H₃₁N₃O₅SiI: 572.1073. Found [M+H]⁺: 572.1084.

### Example 1.4 4-N-Benzoyl-2′-O-(tert-butyldimethylsilyl)-3′-deoxy-3′,4′-didehydrocytidine (5)

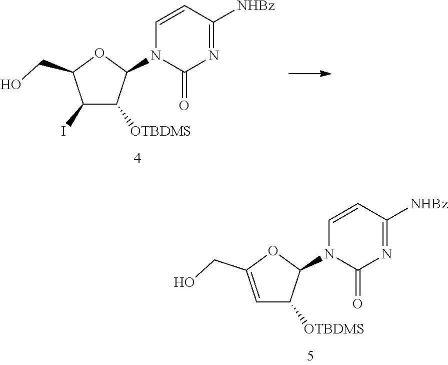

To a suspension of iodide 4 (2.75 g, 4.81 mmol) in PhMe (96 mL) was added DABCO (1.91 g, 16.8 mmol), then the resulting suspension was heated to 75° C. and stirred for 18 h. The reaction mixture was cooled to room temperature and filtered to remove precipitated salts, then the filtrate was concentrated in vacuo. The crude product was purified by flash column chromatography (80 g silica gel, 40%-100% EtOAc-Hex) to afford the title compound 5 (2.00 g, 94%) as a colourless solid. [α]_{D}²⁰−60.0 (0.25, CHCl₃); ¹H NMR (500 MHZ, CDCl₃) δ 8.72 (s, 1H), 7.94-7.81 (m, 2H), 7.68-7.57 (m, 2H), 7.56-7.42 (m, 3H), 6.36 (d, J=1.5 Hz, 1H), 5.21 (d, J=2.4 Hz, 1H), 4.86 (br s, 1H), 4.41-4.30 (m, 2H), 0.90 (s, 9H,), 0.16 (s, 3H), 0.11 (s, 3H); ¹³C NMR (126 MHZ, CDCl₃) δ 166.9, 162.8, 161.4, 154.9, 143.8, 133.2, 133.0, 129.0, 127.8, 101.5, 97.3, 94.3, 80.8, 57.4, 25.8, 18.2, −4.5, −4.7; HRMS (ESI+): Calculated for C₂₂H₃₀N₃O₅Si: 444.1949. Found [M+H]⁺: 444.1953.

### Example 1.5 2′-O-(tert-Butyldimethylsilyl)-3′-deoxy-3′,4′-didehydrocytidine (6)

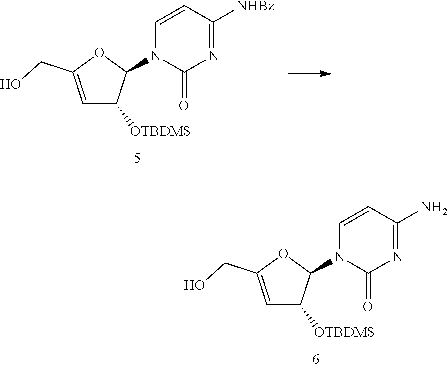

Benzamide 5 (500 mg, 1.13 mmol) was dissolved in a solution of NH₃ (7 M in MeOH, 11.3 mL, 79.1 mmol) and the resulting mixture was stirred at room temperature for 3 h. The reaction mixture was concentrated in vacuo and the crude oil triturated with a small quantity of EtOAc. The precipitate was collected by vacuum filtration, washing with a small quantity of Et20 to afford the title compound 6 (297 mg, 78%) as a colourless solid. ¹H NMR (500 MHZ, DMSO-d6) δ 7.27 (br s, 1H), 7.25 (d, J=7.4 Hz, 1H), 7.19 (br s, 1H), 6.18 (d, J=2.2 Hz, 1H), 5.73 (d, J=7.4 Hz, 1H), 5.28 (t, J=5.8 Hz, 1H), 5.12-5.09 (m, 1H), 4.88-4.85 (m, 1H), 4.04 (d, J=4.9 Hz, 2H), 0.84 (s, 9H), 0.06 (s, 3H), 0.04 (s, 3H); ¹³C NMR (126 MHz, DMSO-d6) δ 165.6, 162.3, 154.6, 140.3, 99.6, 94.9, 93.2, 79.9, 56.1, 25.7, 17.7, −4.7, −4.8; HRMS (ESI+): Calculated for C15H25N304NaSi: 362.1507. Found [M+Na]⁺: 362.1507.

### Example 2: Preparation of 3′-deoxy-3′,4′-didehydro-5′-0-isobutyroyl-cytidine (8)

### Example 2.1 2′-O-(tert-Butyldimethylsilyl)-3′-deoxy-3′,4′-didehydro-5′-O-isobutyroyl-cytidine (7)

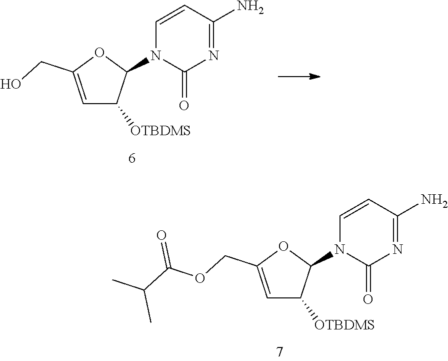 [SMILES: CC(C)(C)[Si](C)(C)O[C@@H]1C=C(CO)O[C@H]1n1ccc(N)nc1=O ; CC(C)C(=O)OCC1=C[C@@H](O[Si](C)(C)C(C)(C)C)[C@H](n2ccc(N)nc2=O)O1]

To a solution of alcohol 6 (109 mg, 0.321 mmol) in anhydrous MeCN (1 mL) was added DMAP (10 mg, 79 μmol), followed by DBU (110 μL, 0.715 mmol). The mixture was stirred for 5 min, then treated dropwise with isobutyric anhydride (65 L, 0.38 mmol), and stirred at room temperature for 18 h. The reaction mixture was concentrated in vacuo and purified by flash column chromatography (silica gel, 0-5% MeOH-CH₂Cl₂, then isocratic elution with 5% MeOH-CH₂Cl₂) to afford the title compound 7 (109 mg, 83%) as a colorless oil. ¹H NMR (500 MHZ, CDCl3) δ 7.14-7.07 (m, 1H), 6.35-6.31 (m, 1H), 5.82-5.75 (m, 1H), 5.19-5.15 (m, 1H), 4.89-4.84 (m, 1H), 4.73-4.68 (m, 2H), 2.61 (hept, J=7.0 Hz, 1H), 1.18 (d, J=7.0 Hz, 6H), 0.88 (s, 9H), 0.11 (s, 3H), 0.07 (s, 3H); ¹³C NMR (126 MHZ, CDCl₃) δ 176.4, 166.0, 157.0, 155.2, 140.3, 103.0, 95.5, 94.4, 80.5, 58.5, 34.0, 25.9, 19.0, 18.2, −4.3, -4.6; HRMS (ESI+): Calculated for C19H31N₃O₅NaSi: 432.1925. Found [M+Na]⁺: 432.1935.

### Example 2.2 3′-Deoxy-3′,4′-didehydro-5′-O-isobutyroyl-cytidine (8)

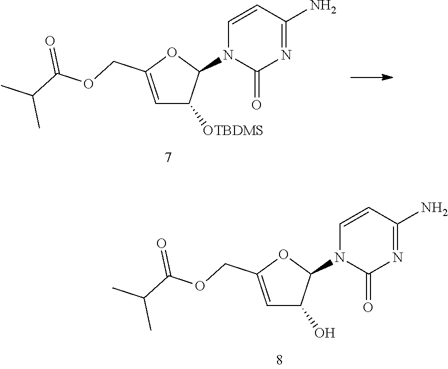 [SMILES: CC(C)C(=O)OCC1=C[C@@H](O[Si](C)(C)C(C)(C)C)[C@H](n2ccc(N)nc2=O)O1 ; CC(C)C(=O)OCC1=C[C@@H](O)[C@H](n2ccc(N)nc2=O)O1]

To a solution of silyl ether 7 (40 mg, 98 μmol) in anhydrous THF (1 mL) at room temperature was added triethylamine trihydrofluoride (20 μL, 0.12 mmol). The reaction was stirred for 24 h, then concentrated in vacuo and purified by flash column chromatography (silica gel, 0-20% EtOH-CH₂Cl₂, then isocratic elution with 20% EtOH-CH₂Cl₂) to afford the title compound 8 (21 mg, 73%) as a colorless oil. ¹H NMR (500 MHZ, MeOD) δ 7.32 (d, J=7.5 Hz, 1H), 6.32 (d, J=2.0 Hz, 1H), 5.89 (d, J=7.5 Hz, 1H), 5.32 (d, J=2.5 Hz, 1H), 4.83-4.81 (m, 1H), 4.77 (s, 2H), 2.62 (hept, J=7.0 Hz, 1H), 1.17 (d, J=7.0 Hz, 6H); ¹³C NMR (126 MHz, MeOD) δ 177.8, 167.7, 158.6, 157.8, 141.5, 103.5, 96.7, 95.3, 80.2, 59.3, 35.1, 19.3; HRMS (ESI+): Calculated for C₁₃H₁₈N₃O₅: 296.1241. Found [M +H]+: 296.1247.

### Example 3: Preparation of 3′-deoxy-3′,4′-didehydro-4-N-hydroxy-5′-O-isobutyroyl-cytidine (10)

### Example 3.1 2′-O-(tert-Butyldimethylsilyl)-3′-deoxy-3′,4′-didehydro-4-N-hydroxy-5′-0-isobutyroyl-cytidine (9)

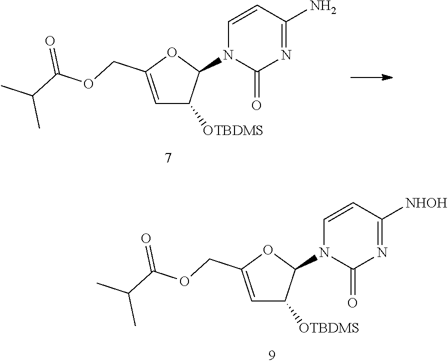

To a solution of amine 7 (43.0 mg, 0.105 mmol) in i-PrOH-H₂O (2:1, 4.3 mL) was added hydroxylammonium sulfate (56 mg, 0.34 mmol) and the resultant mixture was stirred at 78 ° ° C.for 20 h. The reaction mixture was concentrated in vacuo and purified by (silica gel, 0-10% EtOH-CH₂Cl₂, then isocratic elution with 10% EtOH-CH₂Cl₂) to afford the title compound 9 (26 mg, 58%) as a white powder. ¹H NMR (500 MHZ, CDCl₃) δ 8.56 (s, 1H), 7.79 (s, 1H), 6.40 (d, J=8.2 Hz, 1H), 6.31 (d, J=2.4 Hz, 1H), 5.63 (d, J=8.2 Hz, 1H), 5.17 (d, J=2.5 Hz, 1H), 4.92-4.85 (m, 1H), 4.69 (s, 2H), 2.60 (hept, J=7.0 Hz, 1H), 1.19 (d, J=2.0 Hz, 3H), 1.18 (d, J=1.9 Hz, 3H), 0.89 (s, 9H), 0.10 (s, 3H), 0.08 (s, 3H); ¹³C NMR (126 MHz, CDCl₃) δ 176.5, 157.5, 149.1, 145.1, 129.9, 102.6, 99.8, 92.9, 80.1, 58.5, 34.0, 25.9, 19.0, 18.2, −4.45, −4.49; HRMS (ESI+): Calculated for C₁₉H₃₂N₃O₆Si: 426.2055. Found [M+H]⁺: 426.2061.

### Example 3.2 3′-Deoxy-3′,4′-didehydro-4-N-hydroxy-5′-O-isobutyroyl-cytidine (10)

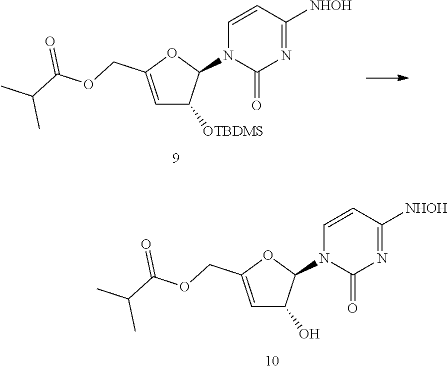

To a solution of silyl ether 9 (26 mg, 61 μmol) in anhydrous THF (0.3 mL) at room temperature was added triethylamine trihydrofluoride (13 μL, 78 μmol). The reaction mixture was stirred for 24 h, then concentrated in vacuo. Purification by flash column chromatography 10 (silica gel, 0-5% MeOH-CH₂Cl₂, then isocratic elution with 5% MeOH-CH₂Cl₂) afforded the title compound (17 mg, 89%) as a white solid. ¹H NMR (500 MHz, MeOD) δ 6.54 (d, J=8.2 Hz, 1H), 6.27 (d, J=2.5 Hz, 1H), 5.61 (d, J=8.2 Hz, 1H), 5.29 (d, J=2.6 Hz, 1H), 4.90-4.85 (m, 1H), 4.72 (s, 2H), 2.61 (hept, J=7.0 Hz, 1H), 1.17 (d, J=7.0 Hz, 6H); ¹³C NMR (126 MHZ, MeOD) δ 177.8, 158.9, 151.0, 145.8, 130.9, 103.1, 100.6, 93.9, 59.3, 35.0, 19.2. HRMS (ESI−): Calculated for C₁₃H₁₆N₃O₆: 310.1045. Found [M−H]⁻: 310.1048.

### Example 4: Preparation of 3′-deoxy-3′,4′-didehydro-4-N-hydroxycytidine (16)

### Example 4.1 2′,5′-Bis-O-(tert-butyldimethylsilyl)uridine (12)

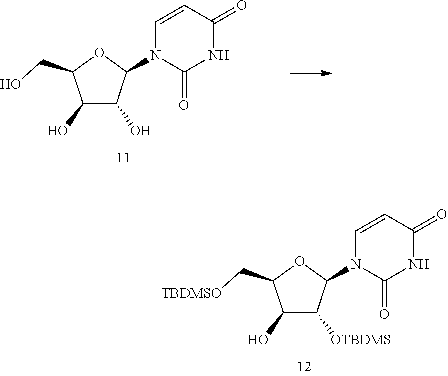 [SMILES: O=c1ccn([C@@H]2O[C@H](CO)[C@H](O)[C@H]2O)c(=O)[nH]1 ; CC(C)(C)[Si](C)(C)OC[C@H]1O[C@@H](n2ccc(=O)[nH]c2=O)[C@H](O[Si](C)(C)C(C)(C)C)[C@H]1O]

The title compound was prepared according to a literature procedure.32 To a solution of uridine (11) (20.0 g, 8.19 mmol) in anhydrous pyridine (16.4 mL) at room temperature was added tert-butyldimethylsilyl chloride (3.82 g, 24.6 mmol). The reaction mixture was stirred for 3 d, then concentrated in vacuo. The resultant oil was dissolved in CHCl₃ (50 mL) and washed with 0.5 M aq HCl (100 mL). The aqueous layer was back-extracted with CHCl₃ (2×50 mL), then the combined organic layers were washed with brine (40 mL), dried over anhydrous MgSO₄, filtered and concentrated in vacuo. Purification by flash column chromatography (silica gel, 5%-60% EtOAc-Hex) afforded the title compound 12 (2.47 g, 63% yield) as a colourless foam. ¹H NMR (500 MHZ, CDCl₃) δ 8.42 (s, 1H), 7.98 (d, J=8.2 Hz, 1H), 5.97 (d, J=4.3 Hz, 1H), 5.69 (dd, J=8.1, 2.3 Hz, 1H), 4.20 (t, J=4.4 Hz, 1H), 4.16-4.07 (m, 2H), 3.99 (dd, J=11.6, 1.8 Hz, 1H), 3.82 (dd, J=11.6, 1.7 Hz, 1H), 2.60 (d, J=5.0 Hz, 1H), 0.94 (s, 9H), 0.91 (s, 9H), 0.14-0.11 (m, 9H), 0.09 (s, 3H).

### Example 4.2 1-(2,5-Bis-O-(tert-butyldimethylsilyl)-3-iodo-ß-D-threo-pentofuranosyl)-uracil (13)

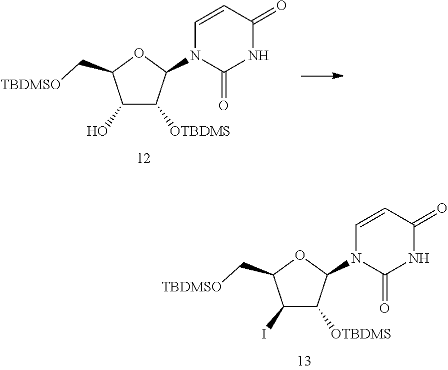 [SMILES: CC(C)(C)[Si](C)(C)OC[C@H]1O[C@@H](n2ccc(=O)[nH]c2=O)[C@H](O[Si](C)(C)C(C)(C)C)[C@@H]1O ; CC(C)(C)[Si](C)(C)OC[C@H]1O[C@@H](n2ccc(=O)[nH]c2=O)[C@H](O[Si](C)(C)C(C)(C)C)[C@H]1I]

To a solution of triphenylphosphine (113 mg, 0.422 mmol), imidazole (58 mg, 0.843 mmol) and I2 (108 mg, 0.426 mmol) in THF (3.0 mL) at room temperature was added a solution of alcohol 12 (100 mg, 0.212 mmol) in THF (2.1 mL). The reaction mixture was heated to 60° C. and stirred for 4 h, then cooled to room temperature. The reaction mixture was diluted with EtOAc (15 mL), then silica gel (750 mg) was added to form a slurry. The slurry was concentrated in vacuo to give a free-flowing solid mixture for purification by dry load; flash column chromatography (25 g silica gel, 5-50% EtOAc-Hex) afforded the title compound 13 (99.8 mg, 81% yield) as a colourless solid. ¹H NMR (500 MHZ, CDCl₃) δ 8.98 (s, 1H), 7.86 (d, J=8.2 Hz, 1H), 5.77 (d, J=3.6 Hz, 1H), 5.74 (d, J=8.2 Hz, 1H), 4.61 (t, J=4.2 Hz, 1H), 4.23 (t, J=5.1 Hz, 1H), 4.06-3.98 (m, 2H), 3.89-3.83 (m, 1H), 0.94 (s, 9H), 0.89 (s, 9H), 0.16 (s, 3H), 0.14 (s, 3H), 0.14 (s, 3H), 0.07 (s, 3H); ¹³C NMR (126 MHz, CDCl₃) δ 163.3, 150.4, 140.5, 102.1, 90.5, 82.6, 80.5, 67.6, 28.7, 26.1, 25.7, 18.5, 17.9, −4.3, −4.7, −5.0, −5.3; HRMS (ESI+): Calculated for C₂₁H₄₀N₂O₅Si₂I: 583.1515. Found [M +H]+: 583.1517.

### Example 4.3 2′,5′-Bis-O-(tert-butyldimethylsilyl)-3′-deoxy-3′,4′-didehydro-uridine (14)

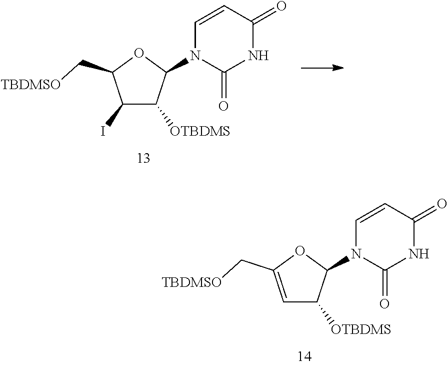 [SMILES: CC(C)(C)[Si](C)(C)OC[C@H]1O[C@@H](n2ccc(=O)[nH]c2=O)[C@H](O[Si](C)(C)C(C)(C)C)[C@H]1I ; CC(C)(C)[Si](C)(C)OCC1=C[C@@H](O[Si](C)(C)C(C)(C)C)[C@H](n2ccc(=O)[nH]c2=O)O1]

To a solution of iodide 13 (250 mg, 0.429 mmol) in PhMe (3 mL) was added DABCO (150 mg, 1.27 mmol), then the reaction mixture was heated to 80° C. and stirred for 18 h. The reaction mixture was cooled down to room temperature and washed with 1 M aq Na2S2O3 solution. The organic layer was then dried over anhydrous MgSO₄, filtered and concentrated in vacuo. Purification by flash column chromatography (silica gel, 0-30% EtOAc/Hex, then isocratic elution with 30% EtOAc/Hex) afforded the title compound 14 (167 mg, 86%) as an off-white crystalline solid. ¹H NMR (500 MHZ, CDCl₃) δ 9.26 (s, 1H), 7.18 (d, J=5.7 Hz, 1H), 6.29-6.25 (m, 1H), 5.69 (d, J=6.4 Hz, 1H), 5.18-5.14 (m, 1H), 4.82-4.78 (m, 1H), 4.26 (s, 2H), 0.91 (s, 9 H), 0.89 (s, 9H), 0.12 (s, 3 H), 0.10 (s, 6 H), 0.08 (s, 3H); ¹³C NMR (126 MHZ, CDCl₃) δ 163.3, 161.8, 150.0, 139.2, 102.9, 101.2, 93.0, 80.5, 58.4, 25.9, 25.8, 18.4, 18.2, −4.5, −4.6, −5.25, −5.31; HRMS (ESI+): Calculated for C₂₁H₃₈N₂O₅NaSi₂: 477.2211. Found [M+Na]⁺: 477.2225.

### Example 4.4 2′,5′-Bis-O-(tert-butyldimethylsilyl)-3′-deoxy-3′,4′-didehydro-4-N-hydroxycytidine (15)

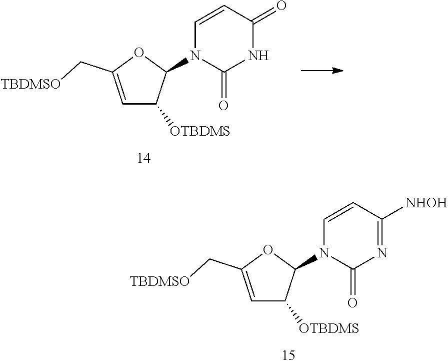

To a solution of uridine 14 (97 mg, 0.21 mmol) in anhydrous CH₂Cl₂ (2.5 mL) at 0° C. was added DMAP (5.0 mg, 41 μmol) followed by DIPEA (200 μL, 1.14 mmol). A solution of 2,4,6-triisopropylbenzenesulfonyl chloride (165 mg, 0.534 mmol) in anhydrous CH₂Cl₂ (2.5 mL) was added dropwise, then the reaction mixture was stirred at room temperature for 18 h. The reaction mixture was cooled down to 0° C. , then DIPEA (160 μL, 0.916 mmol) and hydroxylamine hydrochloride (65 mg, 0.93 mmol) were added sequentially. The reaction was allowed to warm to room temperature and stirred for a further 3 h, then quenched by addition of water and extracted with CH₂Cl₂. The organic extract was washed with brine, dried over anhydrous MgSO₄, filtered and concentrated in vacuo. Purification by flash column chromatography (silica gel, 0-40% EtOAc/Hex, then isocratic elution with 40% EtOAc-Hex) afforded the title compound 15 (71 mg, 71%) as an off-white crystalline solid. ¹H NMR (500 MHz, CDCl₃) δ 8.51 (s, 1H), 7.67 (s, 1H), 6.48 (d, J=8.2 Hz, 1H), 6.28 (d, J=2.1 Hz, 1H), 5.60 (d, J=8.2 Hz, 1H), 5.14-5.09 (m, 1H), 4.87-4.82 (m, 1H), 4.24 (s, 2H), 0.91 (s, 9H), 0.89 (s, 9H), 0.12-0.09 (3s, 9H), 0.08 (s, 3H); ¹³C NMR (126 MHz, CDCI₃) δ 162.2, 149.0, 145.3, 130.1, 100.4, 99.3, 92.8, 80.2, 58.6, 25.9, −4.5, −5.3; HRMS (ESI+): Calculated for C₂₁H₄₀N₃O₅Si₂: 470.2501. Found [M+H]⁺: 470.2511.

### Example 4.5 3′-Deoxy-3′,4′-didehydro-4-N-hydroxycytidine (16)

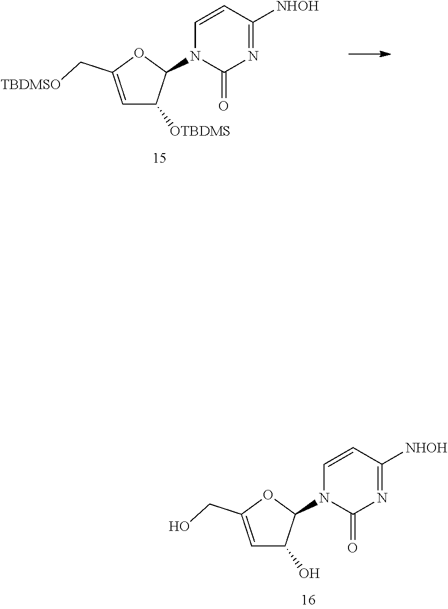

To a solution of silyl ether 15 (50.0 mg, 0.11 mmol) in anhydrous THF (0.5 mL) at room temperature was added triethylamine trihydrofluoride (35 μL, 0.21 mmol). The reaction mixture was stirred for 24 h, then concentrated in vacuo. Purification by flash column chromatography (silica gel, 0-15% MeOH/CH₂Cl₂, then isocratic elution with 15% MeOH/CH₂Cl₂) afforded the title compound 16 (22 mg, 86%) as a white solid. ¹H NMR (500 MHZ, MeOD) δ 6.59 (d, J=8.2 Hz, 1H), 6.26 (d, J=2.4 Hz, 1H), 5.61 (d, J=8.2 Hz, 1H), 5.21 (d, J=2.4 Hz, 1H), 4.88-4.83 (m, 1H), 4.15 (s, 2H); ¹³C NMR (126 MHZ, MeOD) δ 163.9, 151.1, 146.0, 131.0, 100.6, 100.5, 93.9, 79.6, 58.0; HRMS (ESI+): Calculated for C₉H₁₂N₃O₅: 242.0771. Found [M+H]⁺: 242.0781.

### Example 5: Preparation of the S-acyl-2-thioethyl (SATE) phosphotriester derivatives (18)

### Example 5.1 2′-O-(tert-Butyldimethylsilyl)-3′-deoxy-3′,4′-didehydrocytidine-5′-[bis(S-pivaloyl-2-thioethyl)phosphate] (17)

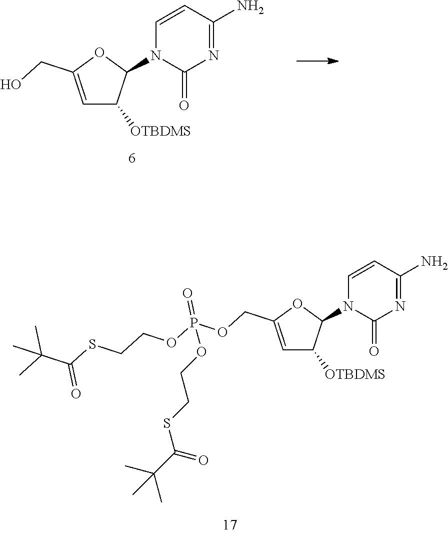 [SMILES: CC(C)(C)[Si](C)(C)O[C@@H]1C=C(CO)O[C@H]1n1ccc(N)nc1=O ; CC(C)(C)C(=O)SCCOP(=O)(OCCSC(=O)C(C)(C)C)OCC1=C[C@@H](O[Si](C)(C)C(C)(C)C)[C@H](n2ccc(N)nc2=O)O1]

To a suspension of alcohol 6 (76.5 mg, 0.225 mmol) and bis(S-pivaloyl-2-thioethyl)-N,N-diisopropylphosphoramidite (129 mg, 0.284 mmol) in MeCN (1.2 mL) at room temperature was added 1H-tetrazole (0.45 M in MeCN, 1.0 mL, 0.45 mmol). The reaction mixture was stirred for 1 h, then cooled to 0° C. and treated dropwise with t-BuOOH (70% w/w in H₂O, 70 μL, 0.51 mmol). The reaction mixture was allowed to warm to room temperature and stirred for 90 min. The reaction mixture was then diluted with EtOAc (20 mL) and washed sequentially with 5% w/w aq Na₂S₂O₃ (5 mL), sat aq NaHCO₃ (10 mL) and brine (5 mL), dried over anhydrous MgSO₄, filtered and concentrated in vacuo. The crude residue was purified by flash column chromatography (silica gel, 60-100% EtOAc-Hex, then 0-10% MeOH-EtOAc) to afford the title compound 17 (161 mg, quant) as a colourless oil. ¹H NMR (500 MHZ, CDCl₃) δ 7.21 (d, J=7.4 Hz, 1H), 6.32 (d, J=2.1 Hz, 1H), 5.80 (d, J=7.4 Hz, 1H), 5.29 (d, J=2.4 Hz, 1H), 4.91 (t, J=2.3 Hz, 1H), 4.71-4.62 (m, 2H), 4.12 (dq, J=8.1, 6.6 Hz, 4H), 3.14 (td, J=6.7, 4.0 Hz, 4H), 1.229 (s, 9H), 1.226 (s, 9H), 0.88 (s, 9H), 0.12 (s, 3H), 0.07 (s, 3H); ¹³C NMR (126 MHZ, CDCl₃) δ 205.9, 205.8, 165.9, 156.2 (d, J=7.1 Hz), 155.3, 140.7, 104.2, 95.3, 94.9, 80.5, 66.6 (d, J=5.4 Hz), 61.9 (d, J=5.2 Hz), 46.7, 28.7 (d, J=7.5 Hz), 28.6 (d, J=7.4 Hz), 27.5, 25.9, 18.2, −4.4, −4.6; 31P NMR (202 MHZ, CDCl₃) δ -1.8; HRMS (ESI+): Calculated for C₂₉H₅₁N₃O₉SiPS₂: 708.2568. Found [M+H]⁺: 708.2565.

### Example 5.2 3′-Deoxy-3′,4′-didehydrocytidine-5′-[bis(S-pivaloyl-2-thioethyl)phosphate] (18)

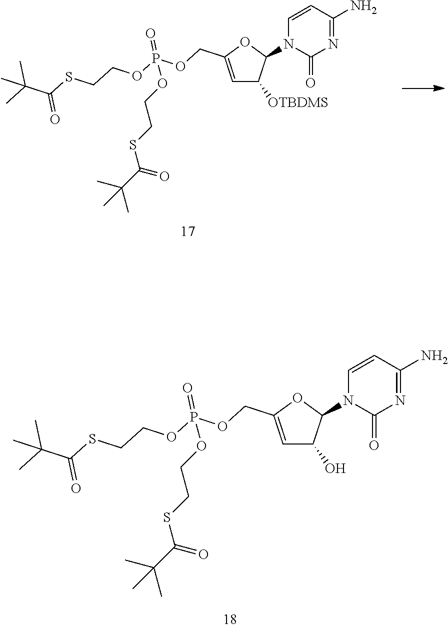 [SMILES: CC(C)(C)C(=O)SCCOP(=O)(OCCSC(=O)C(C)(C)C)OCC1=C[C@@H](O[Si](C)(C)C(C)(C)C)[C@H](n2ccc(N)nc2=O)O1 ; CC(C)(C)C(=O)SCCOP(=O)(OCCSC(=O)C(C)(C)C)OCC1=C[C@@H](O)[C@H](n2ccc(N)nc2=O)O1]

Silyl ether 17 (125 mg, 0.177 mmol) was dissolved in AcOH-H₂O (1:1, 4 mL) and the resultant solution was stirred at room temperature for 42 h, then heated to 40° C. and stirred for a further 3 h. The reaction mixture was then concentrated in vacuo, co-evaporating three times from MeCN to aid removal of AcOH and H₂O. The residue obtained was purified by flash column chromatography (silica gel, 0-40% MeOH-EtOAc) to afford the title compound 18 (105 mg, quant) as a colourless foam. ¹H NMR (500 MHZ, CDCl₃) δ 7.31 (d, J=7.4 Hz, 1H), 6.30 (d, J=2.4 Hz, 1H), 5.91 (d, J=7.5 Hz, 1H), 5.38 (d, J=2.5 Hz, 1H), 4.96 (t, J=2.7 Hz, 1H), 4.72-4.64 (m, 2H), 4.12 (dtd, J=8.1, 6.7, 1.3 Hz, 4H), 3.14 (td, J=6.7, 1.4 Hz, 4H), 1.22 (s, 9H), 1.22 (s, 9H); ¹³C NMR (126 MHZ, CDCl₃) δ 205.9, 205.8, 166.2, 156.3, 155.8 (d, J=7.3 Hz), 139.9, 104.1, 95.8, 95.1, 79.9, 66.6 (d, J=5.8 Hz), 61.9 (d, J=5.2 Hz), 46.69, 28.6 (d, J=7.4 Hz), 27.5; 31P NMR (202 MHZ, CDCl₃) δ -1.99; HRMS (ESI+): Calculated for C₂₃H₂₇N₃O₉PS₂: 594.1703. Found [M+H]⁺: 594.1705.

### Example 6: Preparation of 3′-Deoxy-3′,4′-didehydrocytidine-5′-0-[phenyl(isopropoxy-L-alaninyl) ]-Sp-phosphoramidate (21)

### Example 6.1 2′-O-(tert-Butyldimethylsilyl)-3′-deoxy-3′,4′-didehydrocytidine-5′-0-[phenyl(isopropoxy-L-alaninyl) ]-Sp-phosphoramidate (20)

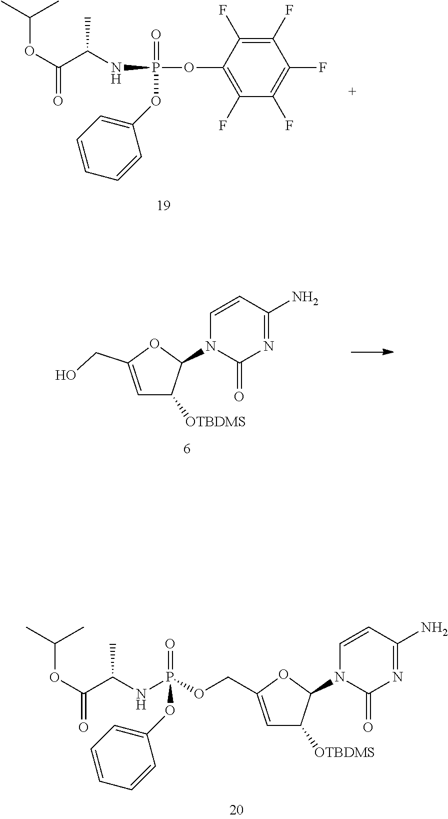 [SMILES: CC(C)OC(=O)[C@H](C)N[P@](=O)(Oc1ccccc1)Oc1c(F)c(F)c(F)c(F)c1F ; CC(C)(C)[Si](C)(C)O[C@@H]1C=C(CO)O[C@H]1n1ccc(N)nc1=O ; CC(C)OC(=O)[C@H](C)N[P@](=O)(OCC1=C[C@@H](O[Si](C)(C)C(C)(C)C)[C@H](n2ccc(N)nc2=O)O1)Oc1ccccc1]

To a solution of alcohol 6 (75 mg, 0.22 mmol) in anhydrous THF (1 mL) at room temperature was added a solution of t-BuMgCl (1.7 mol/L in THF, 0.3 mL, 0.4 mmol) in anhydrous THF (1 mL) dropwise, then the resultant mixture was stirred for 30 min. To the resulting suspension, a solution of pentafluorophenyl reagent 19³³ (160 mg, 0.353 mmol) in anhydrous THF (1 mL) was added dropwise, and the resultant mixture was stirred at room temperature under argon for 20 h. The reaction was quenched by addition of MeOH (2 mL) and concentrated in vacuo. The crude residue was purified by flash column chromatography (silica gel, 0-10% MeOH/CH₂Cl₂, then 10% MeOH/CH₂Cl₂) to afford the title compound 20 as a colourless oil (106 mg, 79%). Stereochemistry at the phosphorus centre was assigned based on the stereochemistry of the pentafluorophenyl reagent.36 1H NMR (500 MHZ, MeOD) δ 7.39-7.15 (m, 6H), 6.34-6.29 (m, 1H), 5.86 (d, J=7.4 Hz, 1H), 5.32-5.27 (m, 1H), 5.02-4.92 (m, 2H), 4.73 (d, J=8.0 Hz, 2H), 3.96-3.86 (m, 1H), 1.35 (d, J=7.1 Hz, 3H), 1.22 (d, J=6.3 Hz, 6H), 0.89 (s, 9H), 0.11 (s, 3H), 0.08 (s, 3H); ¹³C NMR (126 MHZ, MeOD) δ 174.3 (d, J=5.1 Hz), 167.7, 158.0 (d, J=8.2 Hz), 157.6, 152.2 (d, J=7.0 Hz), 141.8, 130.8, 126.2, 121.4 (d, J=4.8 Hz), 104.6, 96.9, 95.7, 81.8, 70.2, 61.9 (d, J=4.9 Hz), 51.7, 26.2, 22.0 (d, J=12.2 Hz), 20.4 (d, J=6.5 Hz)18.9, −4.49, −4.54; 31P NMR (202 MHZ, MeOD) δ 3.6; HRMS (ESI+): Calculated for C₂₇H₄₁N₄NaO₈PSi: 631.2323. Found [M+Na]⁺: 631.2327.

### Example 6.2 3′-Deoxy-3′,4′-didehydrocytidine-5′-O-[phenyl(isopropoxy-L-alaninyl) ]-Sp-phosphoramidate (ddhC-phosphoramidate) (21)

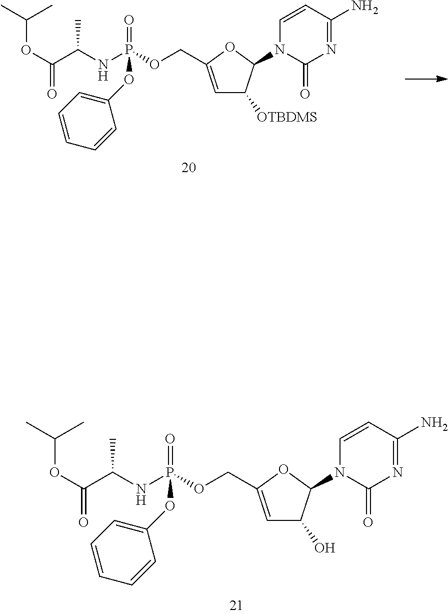 [SMILES: CC(C)OC(=O)[C@H](C)N[P@](=O)(OCC1=C[C@@H](O[Si](C)(C)C(C)(C)C)[C@H](n2ccc(N)nc2=O)O1)Oc1ccccc1 ; CC(C)OC(=O)[C@H](C)N[P@](=O)(OCC1=C[C@@H](O)[C@H](n2ccc(N)nc2=O)O1)Oc1ccccc1]

Compound 20 (78 mg, 0.13 mmol) was dissolved in AcOH-H₂O (1:1, 10 mL) and the resultant solution was stirred at room temperature for 24 h. The reaction mixture was then concentrated in vacuo. The residue obtained was purified by flash column chromatography (silica gel, 5-20% MeOH/CH₂Cl₂) to afford the title compound 21 (56 mg, 88%) as a colourless oil. ¹H NMR (500 MHZ, MeOD) δ 7.39-7.17 (m, 6H), 6.32 (d, J=2.2 Hz, 1H), 5.85 (d, J=7.4 Hz, 1H), 5.36 (d, J=2.6 Hz, 1H), 4.97 (hept, J=6.3 Hz, 1H), 4.83-4.81 (m, 1H), 4.78-4.68 (m, 2H), 3.96-3.86 (m, 1H), 1.35 (d, J=7.1 Hz, 3H), 1.22 (d, J=6.3 Hz, 6H); ¹³C NMR (126 MHZ, MeOD) δ 174.4 (d, J=5.6 Hz), 167.7, 158.1 (d, J=8.4 Hz), 157.8, 152.2 (d, J=7.2 Hz), 141.6, 130.8, 126.2, 121.4 (d, J=4.8 Hz), 104.4, 96.8, 95.6, 80.3, 70.2, 61.9 (d, J=4.7 Hz), 51.7, 21.9 (d, J=10.8 Hz), 20.4 (d, J=6.6 Hz); 31P NMR (202 MHZ, MeOD) δ 3.6; HRMS (ESI+): Calculated for C₂₁H₂₇N₄NaO₈P: 517.1459. Found [M+Na]⁺: 517.1469.

### Example 7: Preparation of 5′-O-acetoyl-3′,4′-didehydro-3′-deoxycytidine (23)

### Example 7.1 5′-Acetoyl-2′-O-tert-butyldimethylsilyl-3′,4′-didehydro-3′-deoxycytidine (22)

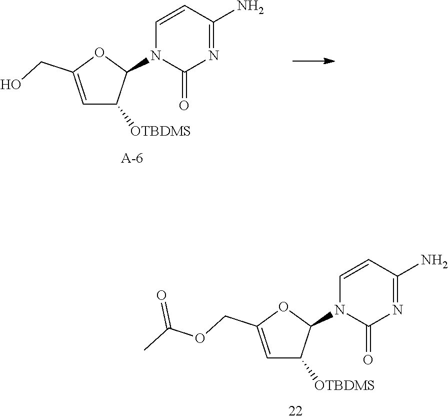

To 2′-O-tert-butyldimethylsilyl-3′,4′-didehydro-3′-deoxycytidine (A-6) (68 mg, 0.20 mmol) and DMAP (5 mg, 0.04 mmol) at room temperature was added DMF (0.9 mL), followed by DBU (0.06 mL, 0.4 mmol), then Ac2O (21 μL, 0.22 mmol). The reaction was stirred for 11 h, after which the reaction mixture was diluted with EtOAc (10 mL) then washed with aq. NaHCO₃ (3×8 mL). The combined aqueous layers were extracted with EtOAc (2 x 5 mL). The combined organic layers were further washed with brine, dried (MgSO₄), filtered, then concentrated in vacuo to give the crude product as a colourless oil, which was then purified by flash column chromatography on silica gel (0-5% MeOH-CH₂Cl₂ over ten column volumes) to afford the title compound (69 mg, 90%) as a white solid. ¹H NMR (500 MHZ, CDCl₃) δ 7.08 (d, J=7.5 Hz, 1H), 6.32 (d, J=2.1 Hz, 1H), 5.82 (d, J=7.4 Hz, 1H), 5.18 (dt, J=2.3, 1.0 Hz, 1H), 4.89 (t, J=2.3 Hz, 1H), 4.75-4.64 (m, 2H), 2.11 (s, 3H), 0.87 (s, 9H), 0.10 (s, 3H), 0.07 (s, 3H); ¹³C NMR (126 MHZ, CDCl₃) δ 170.4, 166.1, 156.7, 155.4, 140.2, 103.1, 95.8, 94.7, 58.7, 26.0, 25.9, 20.8, 18.2, −4.4, −4.6; HRMS (ESI+) Calculated for C17H27N3O₈SiNa: 404.1618. Found [M+Na]⁺: 404.1616.

### Example 7.2 5′-Acetoyl-3′,4′-didehydro-3′-deoxycytidine (23)

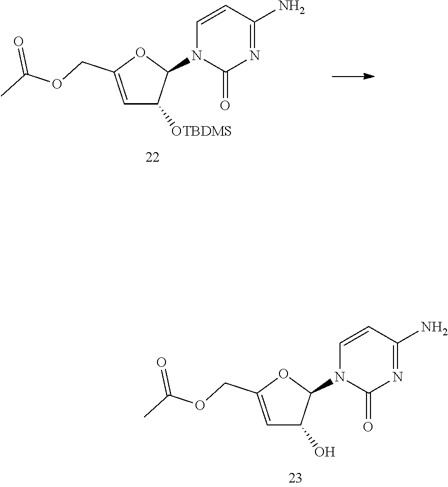 [SMILES: CC(=O)OCC1=C[C@@H](O[Si](C)(C)C(C)(C)C)[C@H](n2ccc(N)nc2=O)O1 ; CC(=O)OCC1=C[C@@H](O)[C@H](n2ccc(N)nc2=O)O1]

To a solution of 5′-acetoyl-2′-O-tert-butyldimethylsilyl-3′,4′-didehydro-3′-deoxycytidine (22) (48 mg, 0.13 mmol) in dry THF (1 mL) at room temperature was added 3HF·Et₃N (25 μL, 0.15 mmol) and the reaction mixture was stirred for 48 h. The reaction mixture was concentrated in vacuo to furnish a colourless oil, which was then purified by flash column chromatography on silica gel (0-10% MeOH-CH₂Cl₂ over ten column volumes) to afford the title compound (30 mg, 89%) as a white solid. ¹H NMR (500 MHZ, DMSO) § 7.28 (s, 1H), 7.22 (s, 1H), 7.21 (d, J=7.4 Hz, 1H), 6.22 (d, J=2.6 Hz, 1H), 5.74 (d, J=7.4 Hz, 1H), 5.65 (d, J=6.2 Hz, 1H), 5.27 (dd, J=2.4, 1.1 Hz, 1H), 4.75-4.71 (m, 1H), 4.71-4.63 (m, 2H), 2.07 (s, 3H); ¹³C NMR (126 MHZ, DMSO) δ 169.8, 165.6, 155.5, 154.6, 140.4, 103.3, 95.0, 93.3, 77.8, 57.9, 20.4; HRMS (ESI+) Calculated for C₁₁H₁₃N₃O₅Na: 290.0753. Found [M+Na]⁺: 290.0758.

### Example 8: Preparation of 3′,4′-didehydro-3′-deoxy-5′-O-pivaloylcytidine (25)

### Example 8.1 2′-O-tert-Butyldimethylsilyl-3′,4′-didehydro-3′-deoxy-5′-pivaloylcytidine (24)

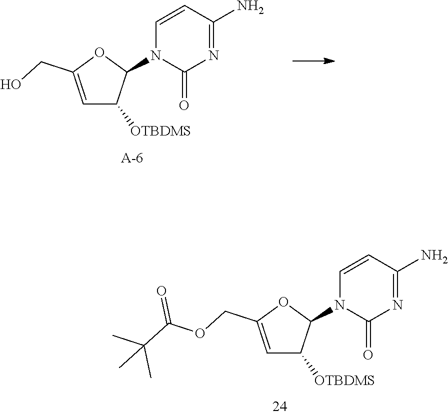

To 2′-O-tert-butyldimethylsilyl-3′,4′-didehydro-3′-deoxycytidine (A-6) (69 mg, 0.20 mmol) and DMAP (5 mg, 0.04 mmol) at room temperature was added DMF (0.9 mL), followed by DBU (0.06 mL, 0.4 mmol), then pivalic anhydride (46 μL, 0.22 mmol). The reaction was stirred for 11 h after which the reaction mixture was diluted with EtOAc (10 mL) and washed with aq. NaHCO₃ (3×8 mL). The collected aqueous layers were extracted with EtOAc (2×5 mL). The combined organic fractions were further washed with brine, dried (MgSO₄), filtered, then concentrated to give the crude product, which was then purified by flash column chromatography on silica gel (0-5% MeOH-CH₂Cl₂ over ten column volumes) to afford the title compound (69 mg, 90%) as a colourless solid. ¹H NMR (500 MHZ, CDCl₃) δ 7.11 (d, J=7.5 Hz, 1H), 6.34 (d, J=1.9 Hz, 1H), 5.78 (d, J=7.4 Hz, 1H), 5.16 (dd, J=2.4, 1.1 Hz, 1H), 4.85 (t, J=2.1 Hz, 1H), 4.75-4.65 (m, 2H), 1.22 (s, 9H), 0.88 (s, 9H), 0.11 (s, 3H), 0.07 (s, 3H); ¹³C NMR (126 MHZ, CDCl₃) δ 177.9, 166.0, 157.1, 155.4, 140.2, 102.9, 95.4, 94.3, 80.6, 58.7, 39.0, 27.3, 25.9, 18.2, −4.3, −4.6. HRMS (ESI+) Calculated for C20H33N3OsSiNa: 446.2087. Found [M+Na]⁺: 446.2082.

### Example 8.2 3′,4′-Didehydro-3′-deoxy-5′-pivaloylcytidine (25)

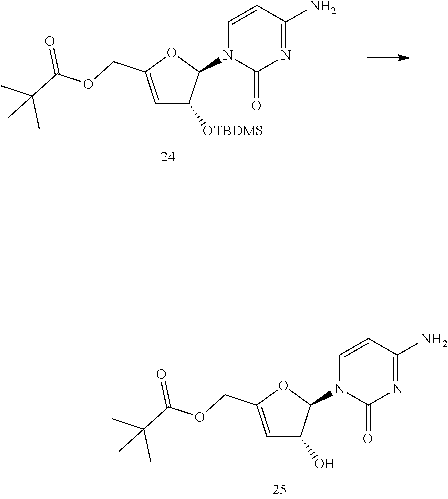 [SMILES: CC(C)(C)C(=O)OCC1=C[C@@H](O[Si](C)(C)C(C)(C)C)[C@H](n2ccc(N)nc2=O)O1 ; CC(C)(C)C(=O)OCC1=C[C@@H](O)[C@H](n2ccc(N)nc2=O)O1]

To a solution of 2′-O-tert-butyldimethylsilyl-3′,4′-didehydro-3′-deoxy-5′-pivaloylcytidine (24) (38 mg, 0.090 mmol) in dry THF (1 mL) at room temperature was added 3HF·Et₃N (18 μL, 0.11 mmol) and the reaction mixture was stirred for 48 h. The reaction mixture was concentrated in vacuo to furnish a yellow oil which was then purified by flash column chromatography on silica gel (0-10% MeOH-CH₂Cl₂ over ten column volumes) to the title compound (24 mg, 87%) as a colourless solid. ¹H NMR (500 MHZ, DMSO) δ 7.26 (s, 1H), 7.23 (s, 1H), 7.18 (d, J=7.4 Hz, 1H), 6.23 (d, J=2.3 Hz, 1H), 5.72 (d, J=7.4 Hz, 1H), 5.66 (d, J=6.2 Hz, 1H), 5.28-5.24 (m, 1H), 4.75-4.65 (m, 3H), 1.15 (s, 9H); ¹³C NMR (126 MHZ, DMSO) δ 176.7, 165.6, 155.8, 154.5, 140.1, 103.0, 94.8, 93.0, 77.8, 58.0, 38.2, 26.7; HRMS (ESI+) Calculated for C₁₄H₁₉N₃O₅Na: 332.1222. Found [M+Na]⁺: 332.1232.

### Example 9: Preparation of 3′,4′-didehydro-3′-deoxy-5′-O-hexanoylcytidine (27)

### Example 9.1 2′-O-tert-Butyldimethylsilyl-3′,4′-didehydro-3′-deoxy-5′-hexanoylcytidine (26)

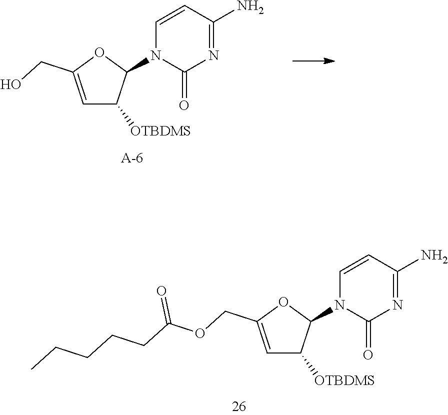

To 2′-O-tert-butyldimethylsilyl-3′,4′-didehydro-3′-deoxycytidine (A-6) (74 mg, 0.22 mmol) and DMAP (5 mg, 0.04 mmol) at room temperature was added DMF (0.9 mL), followed by DBU (0.06 mL, 0.4 mmol), then hexanoic anhydride (57 μL, 0.24 mmol). The reaction was stirred for 11 h after which the reaction mixture was diluted with EtOAc (10 mL), then washed with aq. NaHCO₃ (3×8 mL). The collected aqueous layers were extracted with EtOAc (2×5 mL). The combined organic fractions were further washed with brine, dried over anhydrous MgSO₄, filtered and concentrated in vacuo. Purification by flash column chromatography on silica gel (0-5% MeOH in CH₂Cl₂ over ten column volumes) afforded the title compound (72 mg, 72%) as a colourless solid. ¹H NMR (500 MHZ, CDCl₃) δ 7.08 (d, J=7.4 Hz, 1H), 6.32 (d, J=2.0 Hz, 1H), 5.82 (d, J=7.4 Hz, 1H), 5.19-5.12 (m, 1H), 4.88 (t, J=2.3 Hz, 1H), 4.76-4.64 (m, 2H), 2.35 (t, J=7.5 Hz, 2H), 1.68-1.58 (m, 2H), 1.37-1.23 (m, 4H), 0.88 (m, 3H), 0.87 (s, 9H), 0.10 (s, 3H), 0.07 (s, 3H); ¹³C NMR (126 MHZ, CDCl₃) δ 173.2, 166.1, 156.9, 155.4, 140.2, 102.9, 95.7, 94.6, 80.5, 58.5, 34.1, 31.4, 25.9, 24.7, 22.4, 18.2, 14.0, −4.4, −4.6; HRMS (ESI+) Calculated for C₂₁H₃₅N₃O₅SiNa: 460.2244. Found [M+Na]⁺: 460.2248.

### Example 9.2 3′,4′-Didehydro-3′-deoxy-5′-hexanoylcytidine (27)

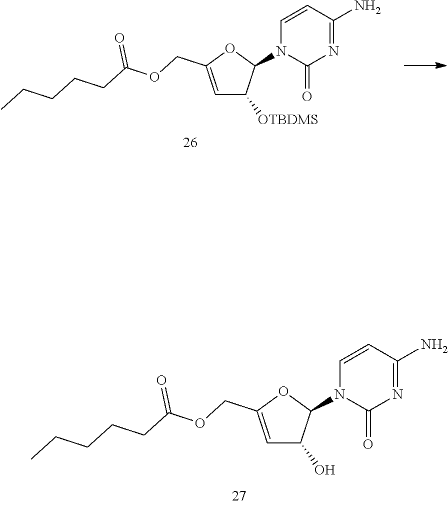 [SMILES: CCCCCC(=O)OCC1=C[C@@H](O[Si](C)(C)C(C)(C)C)[C@H](n2ccc(N)nc2=O)O1 ; CCCCCC(=O)OCC1=C[C@@H](O)[C@H](n2ccc(N)nc2=O)O1]

To a solution of the 2′-O-tert-butyldimethylsilyl-3′,4′-didehydro-3′-deoxy-5′-hexanoylcytidine (26) (43 mg, 98 μmol) in THF (1 mL) at room temperature was added 3HF·Et₃N (20 μL, 0.12 mmol). The reaction mixture was stirred for 48 h, then concentrated in vacuo. Purification by flash column chromatography on silica gel (0-10% MeOH in CH₂Cl₂ over ten column volumes) afforded the title compound (31 mg, 98%) as a colourless solid. ¹H NMR (500 MHZ, CDCl₃) δ 7.21-7.14 (m, 1H), 6.32-6.24 (m, 1H), 5.93-5.84 (m, 1H), 5.26 (t, J=1.9 Hz, 1H), 4.95 (t, J=2.4 Hz, 1H), 4.74-4.64 (m, 2H), 2.34 (t, J=7.3 Hz, 2H), 1.67-1.56 (m, 2H), 1.36-1.23 (m, 4H), 0.91-0.84 (m, 3H); ¹³C NMR (126 MHZ, CDCl₃) δ 173.4, 166.2, 156.6, 156.4, 139.7, 102.7, 96.2, 94.7, 79.7, 58.4, 34.1, 31.4, 24.6, 22.4, 14.0; HRMS (ESI+) Calculated for C₁₅H₂₁N₃O₅Na: 346.1379. Found [M+Na]⁺: 346.1381.

### Example 10: Preparation of 3′,4′-didehydro-3′-deoxy-5′-O-L-valinoylcytidine (30)

### Example 10.1 2′-O-tert-Butyldimethylsilyl-3′,4′-didehydro-3′-deoxy-5′-0-[(N-fluorenylmethoxycarbonyl)-L-valinoyl ]cytidine (28)

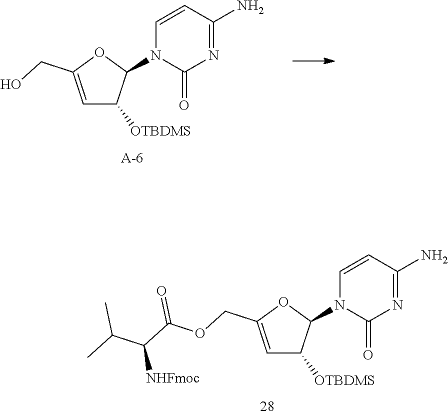

To a solution of 2′-O-tert-butyldimethylsilyl-3′,4′-didehydro-3′-deoxycytidine (A-6) (51.4 mg, 0.151 mmol), N-(fluorenylmethoxycarbonyl)-L-valine (79 mg, 0.23 mmol) and triphenylphosphine (101 mg, 0.377 mmol) in THF (0.8 mL) at 0° C. was added DIAD (60 μL, 0.30 mmol) dropwise. The reaction mixture was allowed to warm to room temperature and stirred for 1 h, then partitioned between EtOAc (20 mL) and water (5 mL). The organic phase was washed with sat aq NaHCO₃ (5 mL), brine (5 mL), dried over anhydrous MgSO₄, filtered and concentrated in vacuo. The crude residue was partially purified by flash column chromatography on silica gel (40-100% EtOAc-Hex) to afford the title compound contaminated with triphenylphosphine oxide (150 mg) as a colourless solid, which was used directly in the next step. A small sample was purified further under the same chromatography conditions for characterisation. ¹H NMR (500 MHZ, CDCl₃) δ 7.78-7.73 (m, 2H), 7.62-7.56 (m, 2H), 7.39 (td, J=7.5, 2.5 Hz, 2H), 7.33-7.28 (m, 2H), 7.05 (d, J=7.5 Hz, 1H), 6.31 (d, J=2.2 Hz, 1H), 5.73 (d, J=7.5 Hz, 1H), 5.35 (d, J=9.1 Hz, 1H), 5.22 (d, J=2.5 Hz, 1H), 4.89 (t, J=2.3 Hz, 1H), 4.78 (s, 2H), 4.45-4.31 (m, 3H), 4.22 (t, J=7.1 Hz, 1H), 2.25-2.12 (m, 1H), 0.98 (d, J=6.8 Hz, 3H), 0.92 (d, J=6.8 Hz, 3H), 0.87 (s, 9H), 0.10 (s, 3H), 0.06 (s, 3H); ¹³C NMR (126 MHz, CDCl₃) d 171.7, 165.9, 156.4, 156.1, 155.3, 144.0, 143.8, 141.5, 140.3, 127.9, 127.2, 125.20, 125.16, 120.2, 103.6, 95.5, 94.7, 80.4, 67.2, 59.3, 59.2, 47.3, 31.3, 25.9, 19.1, 18.2, 17.8, −4.4, −4.6; HRMS (ESI+) Calculated for C₃₅H₄₅N₄O₇Si: 661.3058. Found [M+H]⁺: 661.3066.

### Example 10.2 3′,4′-Didehydro-3′-deoxy-5′-O-[(N-fluorenylmethoxycarbonyl)-L-valinoyl]cytidine (29)

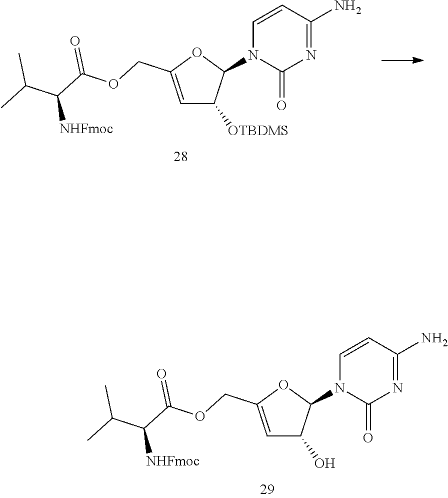

To a solution of the above 2′-O-tert-butyldimethylsilyl-3′,4′-didehydro-3′-deoxy-5′-O-[(N-fluorenylmethoxycarbonyl)-L-valinoyl]cytidine (28) in THF (1.5 mL) was added 3HF·Et₃N (50 μL, 0.30 mmol) and the mixture was stirred at room temperature for 18 h. The reaction mixture was partitioned between EtOAc (20 mL) and water (5 mL), then the organic phase was washed with brine (5 mL), dried over anhydrous MgSO₄, filtered and concentrated in vacuo. Purification by flash column chromatography on silica gel (0-40% MeOH-EtOAc) afforded the title compound (606 mg, 73% yield over two steps) as a colourless solid. ¹H NMR (500 MHZ, DMSO-d6) δ 7.89 (d, J=7.5 Hz, 2H), 7.84 (d, J=8.1 Hz, 1H), 7.73 (t, J=7.3 Hz, 2H), 7.41 (t, J=7.5 Hz, 2H), 7.35-7.21 (m, 4H), 7.18 (d, J=7.4 Hz, 1H), 6.22 (d, J=2.7 Hz, 1H), 5.73 (d, J=7.3 Hz, 1H), 5.68 (d, J=6.1 Hz, 1H), 5.30 (d, J=2.8 Hz, 1H), 4.82-4.68 (m, 3H), 4.33-4.26 (m, 2H), 4.23 (t, J=7.2 Hz, 1H), 4.00-3.92 (m, 1H), 2.05 (p, J=6.6 Hz, 1H), 0.94-0.86 (m, 6H); ¹³C NMR (126 MHZ, DMSO-d6) δ 171.3, 165.7, 156.4, 155.3, 154.6, 143.8, 143.7, 140.7, 140.4, 127.7, 127.1, 125.3, 120.1, 103.5, 95.0, 93.3, 77.8, 65.8, 59.8, 58.3, 46.6, 29.7, 19.0, 18.4; HRMS (ESI+) Calculated for C₂₉H₃₁N₄O₇: 547.2193. Found [M+H]⁺: 547.2194.

### Example 10.3 3′,4′-Didehydro-3′-deoxy-5′-O-L-valinoylcytidine (30)

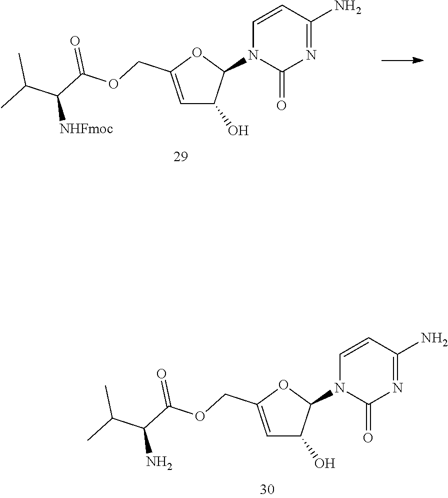

To a solution of 3′,4′-didehydro-3′-deoxy-5′-O-[(N-fluorenylmethoxycarbonyl)-L-valinoyl]cytidine (29) (55 mg, 0.10 mmol) in CH₂Cl₂ (1.0 mL) at δ ° ° C.was added diethylamine (0.2 mL, 2 mmol). The reaction mixture was allowed to warm to room temperature slowly and stirred for 5 h, then concentrated in vacuo. Purification by flash column chromatography on silica gel (0-40% MeOH-EtOAc) afforded the title compound (27.5 mg, 84%) as an off-white solid after lyophilization from MeCN-H₂O. ¹H NMR (500 MHZ, DMSO-d6) δ 7.28 (br s, 1H), 7.23 (br s, 1H), 7.19 (d, J=7.5 Hz, 1H), 6.22 (d, J=2.5 Hz, 1H), 5.72 (d, J=7.4 Hz, 1H), 5.66 (d, J=6.1 Hz, 1H), 5.30 (d, J=2.5 Hz, 1H), 4.82-4.66 (m, 3H), 3.16 (d, J=5.4 Hz, 1H), 1.88-1.79 (m, 1H), 0.87 (d, J=6.9 Hz, 3H), 0.83 (d, J=6.8 Hz, 3H); ¹³C NMR (126 MHZ, DMSO-d6) δ 174.6, 165.6, 155.6, 154.6, 140.3, 103.4, 94.9, 93.2, 77.8, 59.5, 57.9, 31.8, 19.1, 17.4; HRMS (ESI+) Calculated for C₁₄H₂₁N₄O₅: 325.1512. Found [M +H]+: 325.1517.

### Example 11: Preparation of 3′,4′-didehydro-3′-deoxycytidine-5′-0-{phenyl[2-(ethyl)but-1-yloxy-L-alaninyl]}-Sp-phosphoramidate (32)

### Example 11.1 2′-O-tert-Butyldimethylsilyl-3′,4′-didehydro-3′-deoxycytidine-5′-0-{phenyl [2-(ethyl) but-1-yloxy-L-alaninyl] }-Sp-phosphoramidate (31)

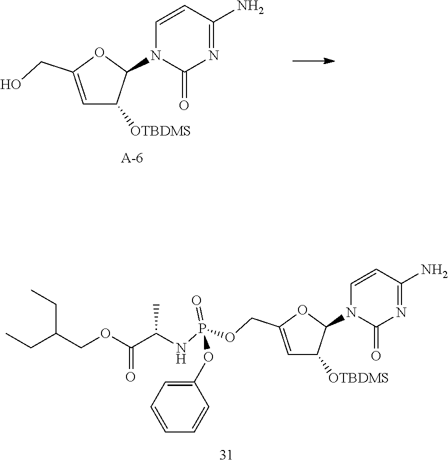

To a solution of 2′-O-tert-butyldimethylsilyl-3′,4′-didehydro-3′-deoxycytidine (A-6) (87 mg, 0.26 mmol) in THF (0.9 mL) at room temperature was added dropwise tert-butylmagnesium chloride (1 M in THF, 0.51 mL, 0.51 mmol). The reaction mixture was stirred for 30 min, then a solution of N-[(S)-(4-nitrophenoxy)phenoxyphosphinyl]-2-ethylbutyl ester L-alanine (182 mg, 0.384 mmol) in THF (0.9 mL) was added dropwise. The reaction mixture was stirred for 12 h, then quenched by addition of sat aq NaHCO₃ (5 mL). The aqueous phase was extracted with EtOAc (3×25 mL), then the combined organic phases were washed with brine, dried over anhydrous MgSO₄, filtered and concentrated in vacuo. Purification by flash column chromatography on silica gel (0-5% MeOH-CH₂Cl₂ over 20 column volumes) afforded the title compound (124 mg, 74%) as a colourless oil. ¹H NMR (500 MHz, CDCl₃) δ 7.31-7.27 (m, 2H), 7.23-7.19 (m, 2H), 7.16-7.09 (m, 2H), 6.08 (d, J=2.4 Hz, 1H), 5.78 (d, J=7.4 Hz, 1H), 5.15-5.10 (m, 1H), 4.98 (t, J=2.5 Hz, 1H), 4.73-4.59 (m, 2H), 4.21 (dd, J=12.1, 9.5 Hz, 1H), 4.12-4.02 (m, 2H), 4.02-3.94 (m, 1H), 1.50 (hept, J=6.2 Hz, 1H), 1.42 (d, J =7.1 Hz, 3H), 1.37-1.28 (m, 4H), 0.87 (t, J=7.6 Hz, 6H), 0.86 (s, 9H), 0.08 (s, 3H), 0.05 (s, 3H); ¹³C NMR (126 MHZ, CDCl₃) δ 174.0 (d, J=6.6 Hz), 166.0, 156.2 (d, J=6.3 Hz), 155.2, 150.8 (d, J=7.1 Hz), 141.4, 129.8, 125.1, 120.4 (d, J=4.6 Hz), 103.8, 96.7, 95.7, 80.3, 67.7, 61.0 (d, J=4.9 Hz), 50.5, 40.4, 25.9, 23.3 (d, J=3.4 Hz), 21.0 (d, J=5.3 Hz), 18.2, 11.1 (d, J=4.9 Hz), -4.4, −4.6; ³¹P NMR (202 MHZ, CDCl₃) δ 2.81; HRMS (ESI+) Calculated for C₃₀H₄₇N₄O₈PSiNa: 673.2798. Found [M+Na]⁺: 673.2808.

### Example 11.2 3′,4′-Didehydro-3′-deoxycytidine-5′-0-{phenyl [2-(ethyl)but-1-yloxy-L-alaninyl] }-Sp-phosphoramidate (32)

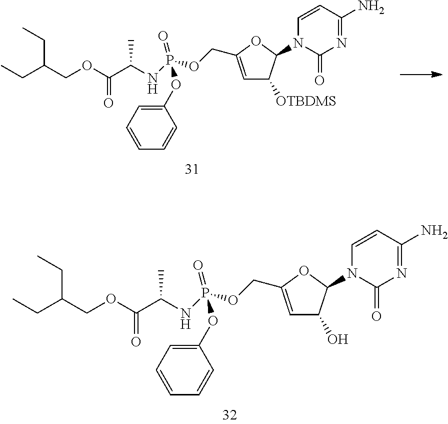 [SMILES: CCC(CC)COC(=O)[C@H](C)N[P@](=O)(OCC1=C[C@@H](O[Si](C)(C)C(C)(C)C)[C@H](n2ccc(N)nc2=O)O1)Oc1ccccc1 ; CCC(CC)COC(=O)[C@H](C)N[P@](=O)(OCC1=C[C@@H](O)[C@H](n2ccc(N)nc2=O)O1)Oc1ccccc1]

2′-O-tert-Butyldimethylsilyl-3′,4′-didehydro-3′-deoxycytidine-5′-O-{phenyl[2-(ethyl)but-1-yloxy-L-alaninyl] }-Sp-phosphoramidite (31) (101 mg, 0.155 mmol) was suspended in AcOH: H₂O (1:1, 13 mL) and stirred at room temperature for 12 h. The reaction mixture was concentrated in vacuo then purified by flash column chromatography on silica gel (0-20% MeOH-CH₂Cl₂ over 24 column volumes) to afford the title compound (52 mg, 62%) as a white solid. ¹H NMR (400 MHZ, MeOD) δ 7.39-7.33 (m, 2H), 7.31 (d, J=7.5 Hz, 1H), 7.28-7.17 (m, 3H), 6.31 (d, J=2.1 Hz, 1H), 5.85 (d, J=7.4 Hz, 1H), 5.38-5.33 (m, 1H), 4.79 (t, J=2.3 Hz, 1H), 4.78-4.68 (m, 2H), 4.10-3.92 (m, 3H), 1.56-1.45 (m, 1H), 1.41-1.31 (m, 7H), 0.89 (t, J=7.5 Hz, 6H); ¹³C NMR (126 MHZ, MeOD) δ 175.0 (d, J=5.2 Hz), 167.7, 158.1 (d, J=8.4 Hz), 157.8, 152.1 (d, J=7.0 Hz), 141.6, 130.8, 126.2, 121.4 (d, J=4.8 Hz), 104.5, 96.8, 95.6, 80.3, 68.2, 61.9 (d, J=4.6 Hz), 51.6, 41.8, 24.3 (d, J=2.7 Hz), 20.5 (d, J=6.8 Hz), 11.3 (d, J=5.8 Hz); ³¹P NMR(162 MHZ, MeOD) δ 3.57; HRMS (ESI+) Calculated for C₂₄H₃₃N₄O₈PNa: 559.1934. Found [M+Na]⁺: 559.1933.

### Example 12: Preparation of3′,4′-didehydro-3′-deoxycytidine-5′-bis(pivaloyloxymethyl)phosphate (35)

### Example 12.1 2′-O-tert-Butyldimethylsilyl-3′,4′-didehydro-3′-deoxy-4-N-(4,4′-dimethoxytrityl)-cytidine (33)

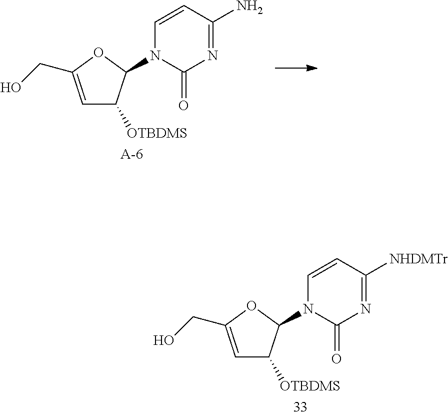

To a solution of 2′-O-tert-butyldimethylsilyl-3′,4′-didehydro-3′-deoxycytidine (A-6) (100 mg, 0.295 mmol) in pyridine (1.5 mL) at room temperature was added chlorotrimethylsilane (80 μL, 0.6 mmol). The reaction mixture was stirred for 45 min, then 4,4′-dimethoxytrityl chloride (126 mg, 0.361 mmol) was added and the mixture left stirring overnight. The reaction was then quenched by addition of NH₄OH (28% w/w in water, 60 μL, 0.4 mmol) and water (1 mL), stirred for 15 min, and diluted with EtOAc (50 mL). The organic layer was washed with 1 M aq HCl (2×20 mL), water (20 mL) and brine (15 mL), dried over anhydrous MgSO₄, filtered and concentrated in vacuo. Purification by flash column chromatography on silica gel (50-100% EtOAc-Hex, then 0-10% MeOH-EtOAc) afforded the title compound (126 mg, 67%) as a pale yellow solid. ¹H NMR (500 MHZ, CDCl₃) δ 7.33-7.24 (m, 3H), 7.23-7.19 (m, 2H), 7.15-7.09 (m, 4H), 6.87 (d, J=7.7 Hz, 1H), 6.85-6.79 (m, 4H), 6.23 (d, J=1.9 Hz, 1H), 5.10 (dd, J=2.5, 1.2 Hz, 1H), 5.02 (d, J=7.6 Hz, 1H), 4.85 (d, J=2.2 Hz, 1H), 4.23 (s, 2H), 3.80 (s, 6H), 0.88 (s, 9H), 0.13 (s, 3H), 0.08 (s, 3H); ¹³C NMR (126 MHZ, CDCl₃) δ 165.7, 160.6, 158.9, 154.8, 144.7, 140.1, 136.4, 130.0, 128.6, 128.5, 127.6, 113.8, 101.3, 95.1, 95.0, 80.5, 70.3, 58.1, 55.4, 25.9, 18.3, −4.3, −4.6; HRMS (ESI+) Calculated for C₃₆H₄₃N₃O₆NaSi: 664.2819. Found [M+Na]⁺: 664.2827.

### Example 12.2 2′-O-tert-Butyldimethylsilyl-3′,4′-didehydro-3′-deoxy-4-N-(4,4′-dimethoxytrityl)cytidine-5′-bis(pivaloyloxymethyl)phosphate (34)

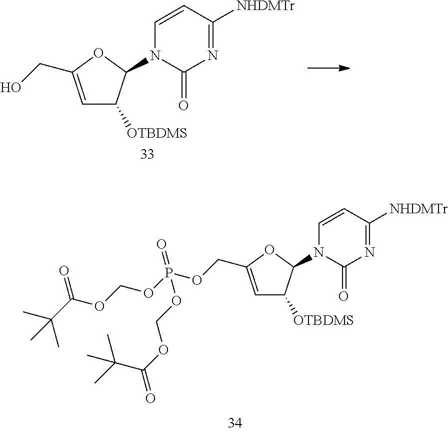

To a solution of oxalyl chloride (0.22 mL, 2.5 mmol) in CH₂Cl₂ (5 mL) at room temperature was added a solution of bis(pivaloyloxymethyl) phosphate (156 mg, 0.500 mmol) and DMF (2 μL, 30 μmol) in CH₂Cl₂ (5 mL). The reaction mixture was stirred for 45 min, then concentrated in vacuo and co-evaporated twice from CH₂Cl₂. The bis(pivaloyloxymethyl) phosphorochloridate (approx. 170 mg, 0.50 mmol) thus obtained was dissolved in THF (1.6 mL), cooled to -78 ºC and treated with Et₃N (0.14 mL, 1.0 mmol), followed by a solution of 2′-O-tert-butyldimethylsilyl-3′,4′-didehydro-3′-deoxy-4-N-(4,4′-dimethoxytrityl)cytidine (33) (106 mg, 0.166 mmol) in THF (1.5 mL) dropwise. The reaction mixture was allowed to warm to room temperature and stirred for 18 h, then diluted with EtOAc (20 mL). The organic phase was washed with sat aq NaHCO₃ (2×10 mL) and brine (5 mL), dried over anhydrous MgSO₄, filtered and concentrated in vacuo. Purification by flash column chromatography on silica gel (20-100% EtOAc-Hex) afforded the title compound (54 mg, 35%) as a colourless foam. ¹H NMR (500 MHZ, CDCl₃) δ 7.34-7.23 (m, 3H), 7.23-7.18 (m, 2H), 7.12 (d, J=8.8 Hz, 4H), 6.86 (d, J=7.7 Hz, 1H), 6.84-6.80 (m, 4H), 6.29 (d, J=2.2 Hz, 1H), 5.63-5.53 (m, 4H), 5.24 (d, J=2.4 Hz, 1H), 5.06 (d, J=7.7 Hz, 1H), 4.86 (d, J=2.5 Hz, 1H), 4.67-4.58 (m, 2H), 3.79 (s, 6H), 1.21 (s, 9H), 1.20 (s, 9H), 0.87 (s, 9H), 0.13 (s, 3H), 0.08 (s, 3H); ¹³C NMR (126 MHz, CDCl₃) δ 5 176.69, 176.67, 165.6, 158.8, 155.5 (d, Jcp =8.4 Hz), 154.7, 144.6, 139.9, 136.39, 136.36, 130.0, 128.6, 128.4, 127.6, 113.7, 104.3, 95.2, 94.9, 82.90 (d, Jcp =4.3 Hz), 82.87 (d, J_{CP}=4.3 Hz), 80.3, 70.3, 62.1 (d, J=4.9 Hz), 55.4, 38.8, 26.9, 25.9, 18.2, −4.4, −4.7; ³¹P NMR (202 MHZ, CDCl₃) δ −4.2; HRMS (ESI+) Calculated for C₄₈H₆₄N₃NaO₁₃SiP: 972.3844. Found [M+Na]⁺: 972.3844.

### Example 12.3 3′,4′-Didehydro-3′-deoxycytidine-5′-bis(pivaloyloxymethyl)phosphate (35)

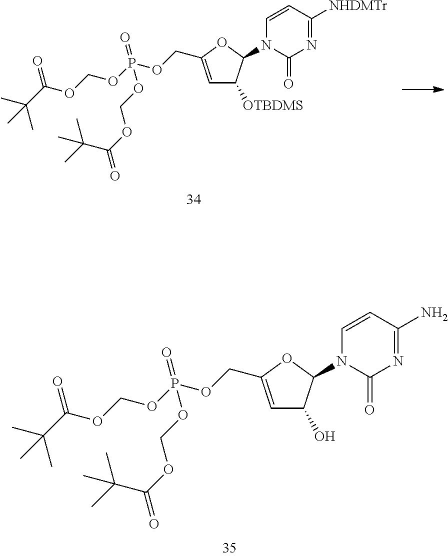

Bis(pivaloyloxymethyl) 2′-O-tert-butyldimethylsilyl-3′,4′-didehydro-3′-deoxy-4-N-(4,4′-dimethoxytrityl)cytidine-5′-phosphate ester (34) (45 mg, 0.047 mmol) was partially dissolved in AcOH-H₂O (1:1, 1.0 mL) and stirred at 50 ºC for 18 h, then concentrated in vacuo. The residue obtained was purified by flash column chromatography on silica gel (0-20% MeOH-CH₂Cl₂) to afford the title compound (10 mg, 40%) as a colourless solid after lyophilization from water. ¹H NMR (500 MHZ, CDCl₃) δ 7.31 (d, J=7.4 Hz, 1H), 6.28 (d, J=2.5 Hz, 1H), 5.91 (d, J=7.4 Hz, 1H), 5.65 (dd, J=13.3, 2.3 Hz, 4H), 5.39 (d, J=2.5 Hz, 1H), 4.97 (t, J=2.6 Hz, 1H), 4.74-4.65 (m, 2H), 1.222 (s, 9H), 1.218 (s, 9H); ¹³C NMR (126 MHZ, CDCl₃) δ 176.9, 165.9, 156.1, 155.3 (d, J_{CP}=7.1 Hz), 140.1, 104.6, 95.7, 95.2, 83.1 (d, J_{CP}=5.1 Hz), 80.0, 62.2 (d, J=4.5 Hz), 38.9, 27.0; 31P NMR (202 MHZ, CDCl₃) δ −4.4; HRMS (ESI+) Calculated for C₂₁H₃₃N₃O₁₁P: 534.1853. Found [M+H]⁺: 534.1865.

### Example 13: Preparation of 3′,4′-didehydro-3′-deoxy-4-N-hydroxycytidine-5′-bis(S-pivaloyl-2-thioethyl)phosphate (38)

### Example 13.1 1-(2′-O-(tert-Butyldimethylsilyl)-3′-iodo-β-D-threo-pentofuranosyl)uracil (A-20)

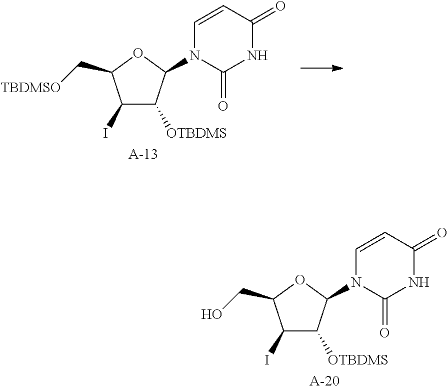

To a solution of 1-(2′,5′-bis-O-(tert-butyldimethylsilyl)-3′-iodo-β-D-threo-pentofuranosyl)-uracil (A-13) (131 mg, 0.225 mmol) in THF (0.5 mL) at 0° C. was added a mixture of TFA-H,O (1:1, 0.14 mL) dropwise over 3 min. The reaction mixture was warmed to room temperature and stirred for 3.5 h. The reaction was neutralised by addition of sat aq NaHCO, (5 mL), then extracted with EtOAc (3×5 mL). The combined organic layers were washed with sat aq NaHCO, (5 mL), brine (5 mL), dried over anhydrous MgSO , filtered and concentrated in vacuo. The crude oil was purified by flash column chromatography (12 g silica gel, 15%-80% EtOAc-Hex) to afford the title compound (90 mg, 85%) as a colourless solid. ¹H NMR (500 MHZ, CDCI) δ 7.78 (d, J=8.2 Hz, 1H), 5.77 (d, J=8.0 Hz, 1H), 5.69 (d, J=3.0 Hz, 1H), 4.71 (t, J=3.5 Hz, 1H), 4.19 (dd, J=5.4, 3.6 Hz, 1H), 4.08 (td, J=5.4, 3.6 Hz, 1H), 4.01 (dd, J=11.8, 5.2 Hz, 1H), 3.85 (dd, J=11.9, 3.9 Hz, 1H), 0.88 (s, 9H), 0.14 (s, 3H), 0.09 (s, 3H); ¹³C NMR (126 MHZ, CDCl₂) δ 163.7, 150.4, 140.9, 102.1, 92.0, 83.4, 80.7, 67.5, 28.5, 25.2, 17.9, −4.4, −4.7; HRMS (ESI+): Calculated for C₁₅H₂₅IN₂O₅SiNa: 491.0475. Found [M+Na]⁺: 491.0475.

### Example 13.2 2′-O-(tert-Butyldimethylsilyl)-3′,4′-didehydro-3′-deoxyuridine (A-21)

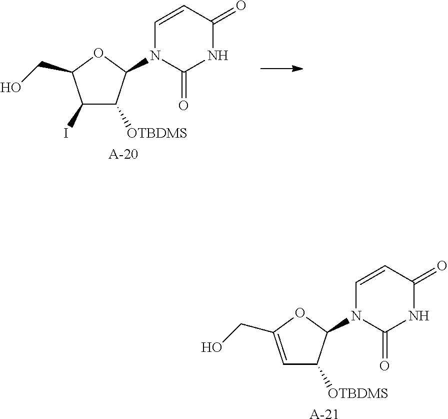

1-(2′-O-(tert-Butyldimethylsilyl)-3′-iodo-β-D-threo-pentofuranosyl)-uracil (A-21) (88 mg, 0.188 mmol) and DABCO (75 mg, 0.66 mmol) were dissolved in PhMe (3.8 mL), then heated to 75° C. and stirred for 18 h. The reaction mixture was cooled to room temperature, then filtered through a pad of Celite®, washing with EtOAc. The filtrate was concentrated in vacuo, then the crude oil obtained was purified by flash column chromatography (silica gel, 50-100% EtOAc-Hex) to afford the title compound (62 mg, 97%) as a colourless foam. ¹H NMR (500 MHZ, CDCI) δ 8.58 (s, 1H), 7.11 (d, J=8.0 Hz, 1H), 6.28 (d, J=2.0 Hz, 1H), 5.80-5.69 (m, 1H), 5.24-5.18 (m, 1H), 4.91 (t, J=2.3 Hz, 1H), 4.29 (d, J=6.3 Hz, 2H), 0.89 (s, 9H), 0.11 (s, 3H), 0.09 (s, 3H); ¹³C NMR (126 MHZ, CDCl₃) δ 162.7, 161.3, 149.8, 139.4, 103.5, 101.2, 93.6, 80.5, 58.0, 25.8, 18.2, −4.5; HRMS (ESI+): Calculated for C₁₅H₂₄N₂O₅SiNa: 363.1352. Found [M+Na]⁺: 363.1350.

### Example 13.3 2′-O-(tert-Butyldimethylsilyl)-3′,4′-didehydro-3′-deoxyuridine-5′-bis(S-pivaloyl-2-thioethyl)phosphate (36)

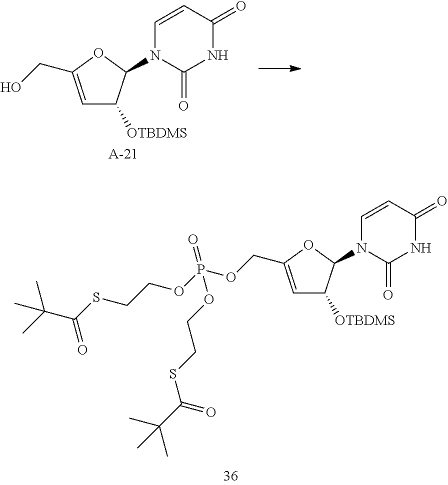

To a solution of 2′-O-(tert-butyldimethylsilyl)-3′,4′-didehydro-3′-deoxyuridine (A-21) (120 mg, 0.353 mmol) in anhydrous MeCN (1.2 mL) at 0° C. was added 1H-tetrazole (0.5 M in MeCN, 1.5 mL, 0.75 mmol), followed by a solution of bis(S-pivaloyl-2-thioethyl)-N,N-diisopropylphosphoramidite (200 mg, 0.441 mmol) in anhydrous MeCN (1.2 mL). The reaction mixture was allowed to warm to room temperature and stirred for 1 h, then cooled back down to 0° C. and tert-butyl hydroperoxide (5.5 M in decane, 130 μL, 0.72 mmol) was added dropwise. The reaction mixture was stirred at room temperature for 1 h, then diluted with EtOAc (20 mL) and washed with 10% aqueous Na₂SO₃, water, and brine. The organic layer was dried over MgSO₄, filtered and concentrated in vacuo. The crude oil was purified by flash column chromatography on silica gel (0-5% MeOH-CH₂Cl₂) to afford the title compound (167 mg, 67%) as a white solid. ¹H NMR (500 MHZ, CDCl₃) δ 7.16 (d, J=8.1 Hz, 1H), 6.32 (d, J=2.2 Hz, 1H), 5.78-5.72 (m, 1H), 5.33 (d, J=2.4 Hz, 1H), 4.94-4.89 (m, 1H), 4.73-4.60 (m, 2H), 4.16-4.07 (m, 4H), 3.16-3.10 (m, 4H), 1.23 (s, 18H), 0.88 (s, 9H), 0.10 (s, 3H), 0.07 (s, 3H); ¹³C NMR (126 MHZ, CDCl₃) δ 205.7, 162.8, 156.6 (d, J=7.2 Hz), 149.8, 139.3, 104.1, 103.6, 93.5, 80.4, 66.6 (d, J=5.7 Hz), 61.6 (d, J=5.0 Hz), 46.7, 28.61 (d, J =7.3 Hz), 28.57 (d, J=6.6 Hz), 27.4, 25.8, 18.2, −4.6; 31P NMR (202 MHZ, CDCl₃) δ −1.8; HRMS (ESI/Q-TOF) Calculated for C₂₉H₄₉N₂NaO₁₀PS₂Si: 731.2228. Found [M+Na]⁺: 731.2242.

### Example 13.4 2′-O-(tert-Butyldimethylsilyl)-3′,4′-didehydro-3′-deoxy-4-N-hydroxycytidine-5′-bis(S-pivaloyl-2-thioethyl) phosphate (37)

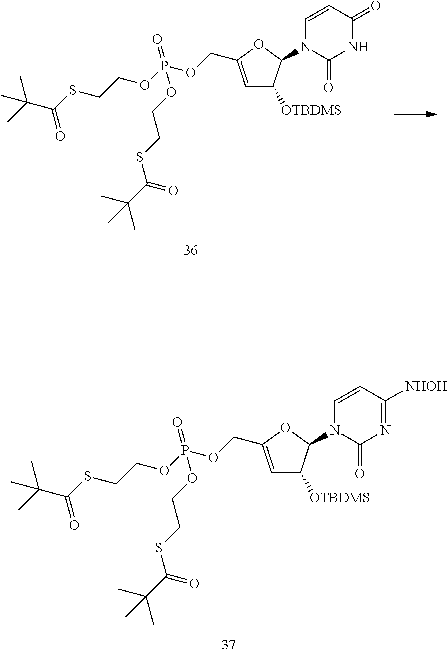

To a solution of 2′-O-(tert-Butyldimethylsilyl)-3′,4′-didehydro-3′-deoxyuridine-5′-[bis(S-pivaloyl-2-thioethyl)phosphate] (36) (130 mg, 0.183 mmol) in anhydrous CH₂Cl₂ (3 mL) at 0° C. was added DMAP (5.0 mg, 41 μmol), followed by DIPEA (160 L, 0.92 mmol). A solution of trityl chloride (145 mg, 0.469 mmol) in anhydrous CH₂Cl₂ (3 mL) was added dropwise and the reaction mixture was stirred at room temperature for 3 h. The reaction mixture was cooled down to 0° C. and DIPEA (130 μL, 0.74 mmol) was added, followed by hydroxylamine hydrochloride (52 mg, 0.74 mmol). The reaction mixture was stirred at room temperature for 2 h, then diluted with water and extracted with CH₂Cl₂. The organic extract was washed with brine, dried over anhydrous MgSO₄, filtered and concentrated in vacuo. The crude oil was purified by flash column chromatography on silica gel (5-20% acetone-CH₂Cl₂) to afford the title compound (22 mg, 17%) as a white solid. ¹H NMR (500 MHZ, CDCl₃) δ 8.60 (s, 1H), 6.46 (d, J=8.2 Hz, 1H), 6.32 (d, J=2.6 Hz, 1H), 5.67 (d, J=8.2 Hz, 1H), 5.28 (d, J=2.3 Hz, 1H), 4.98-4.90 (m, 1H), 4.72-4.56 (m, 2H), 4.17-4.04 (m, 4H), 3.18-3.09 (m, 4H), 1.23 (s, 9H), 1.23 (s, 9H), 0.89 (s, 9H), 0.10 (s, 3H), 0.08 (s, 3H); ¹³C NMR (126 MHz, CDCl₃) δ 205.9, 205.8, 156.8 (d, J=7.4 Hz), 149.0, 144.9, 131.0, 129.8, 129.0, 103.5, 100.1, 93.2, 80.0, 68.3, 66.6 (d, J=4.5 Hz), 61.8 (d, J=4.7 Hz), 46.7, 38.9, 28.7 (d, J=7.2 Hz), 27.5, 25.9, 18.3, −4.5; 31P NMR (202 MHZ, CDCl₃) δ -. 9; HRMS (ESI+): Calculated for C₂₉H₅₀N₃NaO₁₀PS₂Si: 746.2337. Found [M+Na]⁺: 746.2349.

### Example 13.5 3′,4′-Didehydro-3′-deoxy-4-N-hydroxycytidine-5′-bis(S-pivaloyl-2-thioethyl) phosphate (38)

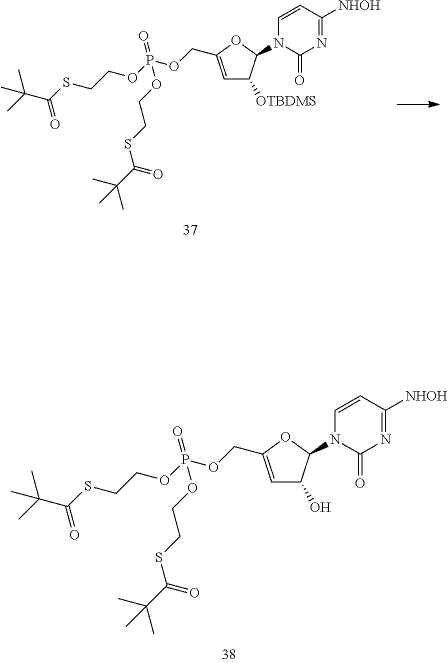

2′-O-(tert-Butyldimethylsilyl)-3′,4′-didehydro-3′-deoxy-4-N-hydroxycytidine-5′-[bis(S-pivaloyl-2-thioethyl)phosphate] (37) (20.0 mg, 27.6 μmol) was stirred with a mixture of acetic acid (2 mL) and water (2 mL) at room temperature for 18 h. The reaction mixture was concentrated in vacuo, then the crude oil was purified by flash column chromatography on silica gel, (0-7% MeOH-CH₂Cl₂) to afford the title compound (8 mg, 48%) as a white solid. ¹H NMR (500 MHZ, CDCl₃) δ 6.48 (d, J=8.2 Hz, 1H), 6.33 (d, J=2.5 Hz, 1H), 5.66 (d, J=8.2 Hz, 1H), 5.40 (d, J=2.4 Hz, 1H), 4.94 (dd, J=2.6, 2.6 Hz, 1H), 4.66 (d, J=9.2 Hz, 2H), 4.12 (td, J=7.1, 7.2 Hz, 4H), 3.14 (t, J=6.7 Hz, 4H), 1.23 (s, 18H); ¹³C NMR (126 MHZ, CDCl₃) δ 206.1, 157.2 (d, J=7.2 Hz), 149.6, 144.9, 129.5, 103.1, 99.9, 93.5, 79.3, 66.6 (d, J=5.8 Hz), 61.7 (d, J=4.7 Hz), 46.7, 28.6 (d, J=7.2 Hz), 27.5; 31P NMR (202 MHZ, CDCl₃) δ -2.0; HRMS (ESI+): Calculated for C₂₃H₃₆N₃NaO₁₀PS₂: 632.1472. Found [M+Na]⁺: 632.1486.

### Example 14: Preparation of 3′,4′-didehydro-2′,3′-dideoxycytidine (A-30) and 3′,4′-didehydro-2′,3′-dideoxy-5′-0-isobutyroylcytidine (39)

### Example 14.1 5′-O-tert-Butyldimethylsilyl-2′-deoxycytidine (A-26)

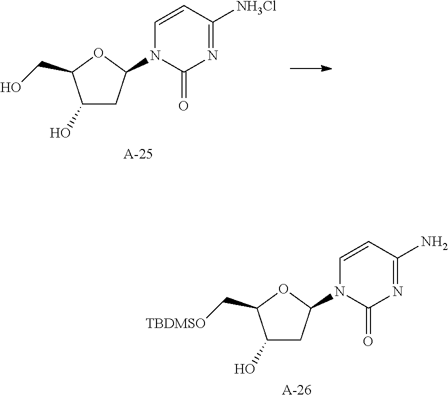

To a solution of 2′-deoxy-cytidine hydrochloride salt (A-25) (3.00 g, 11.4 mmol) and imidazole (1.56 g, 22.7 mmol) in DMF (22 mL) at room temperature was added Et₃N (1.5 mL, 11 mmol), followed by TBDMSCI (1.88 g, 12.1 mmol). The reaction mixture was stirred for 18 h, then concentrated in vacuo, co-evaporating with PhMe. The crude residue was partitioned between CHCl₃ (150 mL) and sat aq NaHCO₃ (50 mL). Upon neutralization of the organic phase, a large amount of the product precipitated from solution - this was collected by vacuum filtration and combined with the organic layer, which were concentrated in vacuo. The residue was re-dissolved in MeOH, then approximately 10 g of silica gel was added, and the slurry was concentrated to dryness in vacuo. Purification by flash column chromatography on silica gel using this dry load (0-20% MeOH-CH₂Cl₂) afforded the title compound (4.14 g) as a solid containing residual solvent. ¹H NMR (500 MHZ, MeOD) δ 8.02 (d, J=7.5 Hz, 1H), 7.68 (d, J=1.1 Hz, 1H), 7.06 (d, J=1.1 Hz, 2H), 6.23 (t, J=6.3 Hz, 1H), 5.85 (d, J=7.5 Hz, 1H), 4.35 (dt, J=6.2, 3.8 Hz, 1H), 3.99-3.82 (m, 3H), 2.39 (ddd, J=13.4, 6.2, 4.0 Hz, 1H), 2.09 (dt, J=13.5, 6.3 Hz, 1H), 0.94 (s, 9H), 0.133 (s, 3H), 0.130 (s, 3H).

### Example 14.2 4-N-3′-O-Dibenzoyl-5′-O-tert-butyldimethylsilyl-2′-deoxycytidine (A-27)

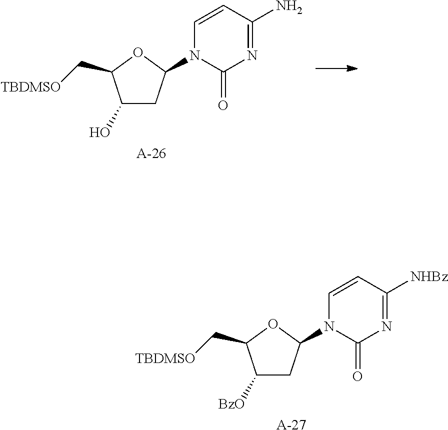

To a solution of 5′-O-tert-butyldimethylsilyl-2′-deoxycytidine (A-26) (3.80 g, 11.1 mmol) and DMAP (70 mg, 0.57 mmol) in pyridine (45 mL) at room temperature was added benzoic anhydride (7.70 g, 33.4 mmol). The reaction mixture was stirred for 18 h, then additional benzoic anhydride (2.57 g, 11.1 mmol) was added and the reaction mixture stirred for a further 24 h. The reaction mixture was concentrated in vacuo, then redissolved in EtOAc (150 mL) and washed with sat aq NaHCO₃ (2×60 mL), water (60 mL) and brine (40 mL). The organic phase was dried over anhydrous MgSO₄, filtered and concentrated in vacuo. Purification by flash column chromatography on silica gel (40-100% EtOAc-Hex) afforded the title compound (5.12 g, 84%) as a colourless solid ¹H NMR (500 MHZ, CDCl₃) δ 8.40 (d, J=7.5 Hz, 1H), 8.13-7.46 (m, 11H), 7.19-7.11 (m, 1H), 6.51 (dd, J=8.1, 5.6 Hz, 1H), 5.53 (d, J=6.3 Hz, 1H), 4.38 (d, J=2.0 Hz, 1H), 4.03 (d, J=2.1 Hz, 2H), 2.94 (dd, J=14.2, 5.6 Hz, 1H), 2.26 (ddd, J=14.2, 8.2, 6.2 Hz, 1H), 0.94 (s, 9H), 0.16 (s, 3H), 0.15 (s, 3H).

### Example 14.3 4-N-3′-O-Dibenzoyl-2′-deoxycytidine (A-28)

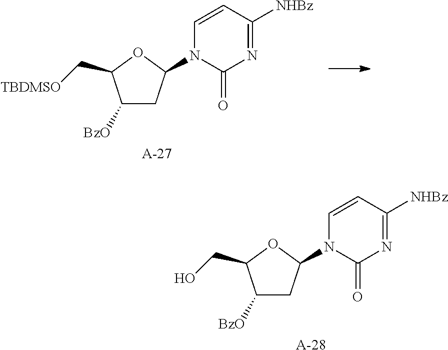

To a solution of 4-N-3′-O-dibenzoyl-5′-O-tert-butyldimethylsilyl-2′-deoxycytidine (A-27) (5.10 g, 9.28 mmol) in THF (25 mL) at 0° C. was added a mixture of TFA-H₂O (1:1, 10 mL). The reaction mixture was allowed to warm to room temperature and stirred for 5 h, then quenched by neutralisation with sat aq NaHCO₃ (100 mL). The precipitate formed was collected by vacuum filtration, then the filtrate was extracted with EtOAc (50 mL). The collected solid and organic phase were combined and concentrated in vacuo to dryness. The resultant solid was suspended in hot MTBE (100 mL), cooled to room temperature, then collected by vacuum filtration to afford the title compound (2.89 g, 72%) as a colourless solid. ¹H NMR (500 MHz, DMSO-d6) d 11.24 (s, 1H), 8.42 (d, J=7.5 Hz, 1H), 8.02 (td, J=7.9, 1.4 Hz, 4H), 7.74-7.68 (m, 1H), 7.66-7.60 (m, 1H), 7.57 (t, J=7.8 Hz, 2H), 7.52 (t, J=7.8 Hz, 2H), 7.40 (d, J=7.2 Hz, 1H), 6.29 (dd, J=8.0, 5.8 Hz, 1H), 5.51 (dt, J=6.3, 2.1 Hz, 1H), 5.25 (t, J=5.3 Hz, 1H), 4.31 (q, J=3.3 Hz, 1H), 3.75 (dd, J=5.3, 3.7 Hz, 2H), 2.70-2.62 (m, 1H), 2.39 (ddd, J=14.2, 8.0, 6.2 Hz, 1H).

### Example 14.4 4-N-Benzoyl-3′,4′-didehydro-2′,3′-dideoxycytidine-5′-aldehyde (A-29)

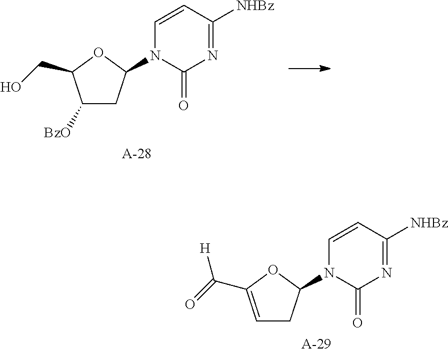

To a suspension of 4-N-3′-O-dibenzoyl-2′-deoxycytidine (A-28) (500 mg, 1.15 mmol) and EDC.HCl (465 mg, 2.30 mmol) in CH₂Cl₂ (4.7 mL) at room temperature was added DMSO (0.4 mL, 6 mmol), followed by a solution of TFA (45 μL, 0.58 mmol) and pyridine (95 L, 1.2 mmol) in CH₂Cl₂ (1.0 mL). The reaction was stirred for 80 min, then diluted with EtOAc (70 mL) and washed sequentially with 1 M aq HCl (20 mL), water (2×15 mL), brine (15 mL), dried over anhydrous MgSO₄, filtered and concentrated in vacuo. The crude intermediate obtained was dissolved in CH₂Cl₂ (8.2 mL), then treated with Et₃N (0.45 mL, 3.2 mmol) and stirred at room temperature for 12 min, then concentrated in vacuo. Insoluble impurities were removed by dissolution of the product in CH₂Cl₂ (40 mL), then filtration of the resulting suspension. The filtrate was concentrated in vacuo, then the residue obtained purified by flash column chromatography on silica gel (0-10% MeOH-CH₂Cl₂) to afford the title compound (168 mg, 47%) as a colourless solid. ¹H NMR (500 MHZ, DMSO-d6) δ 11.29 (s, 1H), 9.49 (s, 1H), 8.03-7.98 (m, 2H), 7.95 (t, J=7.3 Hz, 1H), 7.66-7.58 (m, 1H), 7.55-7.47 (m, 3H), 7.35 (s, 1H), 6.72 (dd, J=9.9, 4.7 Hz, 1H), 6.56 (t, J=3.1 Hz, 1H), 3.47 (ddd, J=20.2, 9.9, 2.9 Hz, 1H), 3.02 (ddd, J=20.2, 4.7, 3.3 Hz, 1H); ¹³C NMR (101 MHZ, DMSO-d6) δ 181.8, 167.6, 163.4, 154.4, 153.9, 145.2, 133.1, 132.8, 128.51, 128.46, 121.8, 97.1, 87.8, 36.6; HRMS (ESI/Q-TOF) Calculated for C₁₇H₁₇N₃NaO₅: 366.1066. Found [M +CH3OH +Na]+: 366.1065.

### Example 14.5 3′,4′-Didehydro-2′,3′-dideoxycytidine (A-30)

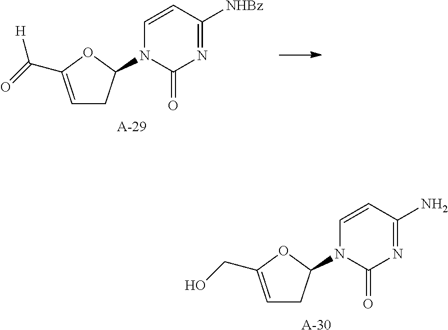

To a solution of 4-N-benzoyl-3′,4′-didehydro-2′,3′-dideoxycytidine-5′-aldehyde (A-29) (155 mg, 0.498 mmol) in MeOH (7 mL) at 0° C. was added NaBH4 (10 mg, 0.51 mmol) and the reaction mixture was stirred at this temperature for 30 min. The reaction was then quenched by addition of acetone (1 mL) and concentrated in vacuo. The residue obtained was suspended in MeOH (5 mL) and treated with MeONa (25% w/w in MeOH, 60 μL, 0.26 mmol), then stirred at room temperature for 2.5 h. The reaction mixture was neutralized by addition of Dowex® 50W X8 H-form resin, then filtered and the resin was washed extensively with MeOH. Silica gel (approx. 300 mg) was added to the filtrate to form a slurry, which was concentrated in vacuo to dryness. The silica dry load was subjected to flash column chromatography on silica gel (0-60% MeOH-CH₂Cl₂) to afford the title compound (48 mg, 46%) as a colourless solid after lyophilization from water. ¹H NMR (500 MHZ, DMSO-d6) δ 7.39 (d, J=7.4 Hz, 1H), 7.21 (brs, 1H), 7.13 (brs, 1H), 6.56 (dd, J=9.4, 3.9 Hz, 1H), 5.74 (d, J=7.4 Hz, 1H), 5.13 (t, J=5.8 Hz, 1H), 4.98 - 4.93 (m, 1H), 4.00 (dd, J=5.7, 1.7 Hz, 2H), 3.14-3.06 (m, 1H), 2.46 (ddt, J=15.1, 3.8, 2.2 Hz, 1H); ¹³C NMR (126 MHZ, DMSO-d6) δ 165.6, 157.1, 154.6, 140.0, 95.1, 94.5, 85.2, 55.9, 36.8; HRMS (ESI+) Calculated for C₉H₁₁N₃NaO₃: 232.0698. Found [M+Na]⁺: 232.0692.

### Example 14.6 3′,4′-Didehydro-2′,3′-dideoxy-5′-O-isobutyroylcytidine (39)

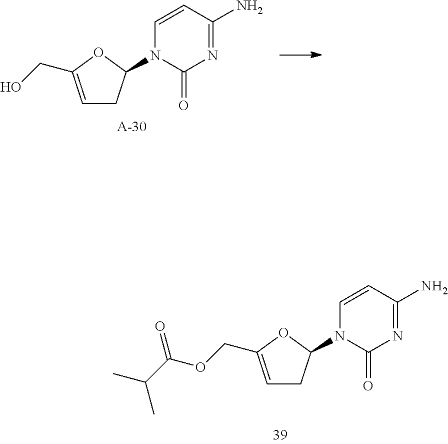

To a solution of 3′,4′-didehydro-2′,3′-dideoxycytidine (A-30) (39 mg, 0.19 mmol) and DMAP (4.5 mg, 36 μmol) in DMF (1.0 mL) at room temperature was added DBU (60 UL, 0.40 mmol) followed by isobutyric anhydride (36 μL, 0.21 mmol). The reaction mixture was stirred for 18 h, then concentrated in vacuo, co-evaporating with PhMe to remove traces of DMF. The residue obtained was purified by flash column chromatography on silica gel (2-15% MeOH-CH₂Cl₂) to afford the title compound (35 mg, 67%) as a colourless solid after lyophilization from water. ¹H NMR (500 MHZ, CDCl₃) δ 7.29 (d, J=7.5 Hz, 1H), 6.68 (dd, J =9.4, 3.9 Hz, 1H), 5.88 (d, J=7.5 Hz, 1H), 5.07-5.04 (m, 1H), 4.65 (s, 2H), 3.28-3.18 (m, 1H), 2.62-2.51 (m, 2H), 1.16 (d, J=7.0 Hz, 6H); ¹³C NMR (126 MHZ, CDCl₃) δ 176.5, 166.2, 155.7, 152.2, 139.7, 99.3, 95.8, 86.3, 58.2, 37.5, 34.0, 19.0; HRMS (ESI+) Calculated for C₁₃H₁₇N₃NaO₄: 302.1117. Found [M+Na]⁺: 302.1122.

### Example 15—Antiviral Materials and Methods

### Example 15.1

### Cell Culture and Virus Strains

Human foreskin fibroblast (HFF) cells prepared from human foreskin tissue were obtained from the University of Alabama at Birmingham tissue procurement facility with approval from its IRB. The tissue was incubated at 4° C. for 4 h in Clinical Medium consisting of minimum essential media (MEM) with Earl's salts supplemented with 10% fetal bovine serum (FBS) (Hyclone, Inc. Logan UT), L-glutamine, fungizone, and vancomycin. Tissue was then placed in phosphate buffered saline (PBS), minced, rinsed to remove the red blood cells, and resuspended in trypsin/EDTA solution. The tissue suspension was incubated at 37° C. and gently agitated to disperse the cells, which were collected by centrifugation. Cells were resuspended in 4 ml Clinical Medium and placed in a 25 cm² flask and incubated at 37° C. in a humidified CO₂ incubator for 24 h. The media was then replaced with fresh Clinical Medium and the cell growth was monitored daily until a confluent monolayer had formed. The HFF cells were then expanded through serial passages in standard growth medium of MEM with Earl's salts supplemented with 10% FBS, L-glutamine, penicillin, and gentamycin. The cells were passaged routinely and used for assays at or below passage ^{10.37,38}

Akata cells were obtained from Louisiana State University, Baton Rouge, LA. The HCMV strain AD169 was obtained from the American Type Culture Collection (ATCC, Manassas, VA). Akata cells latently infected with EBV were obtained from Louisiana State University, Baton Rouge, LA.

### Example 15.2

### Antiviral ASSAYS

Each experiment that evaluates the antiviral activity of compounds includes both positive and negative control compounds to ensure the performance of each assay. Concurrent assessment of cytotoxicity was also performed for each study in the same cell line and with the same compound exposure.

### Example 15.3

### CPE (Cytopathic Effect) Assays for HCMV

Assays were performed in monolayers as described.37 Cells were seeded in 384 well plates and incubated for 24 h to allow the formation of confluent monolayers. Dilutions of test drug were prepared directly in the plates and the monolayers infected at a predetermined MOI based on virus used. After incubation, cytopathology was determined by the addition of CellTiter-Glo (CTG) reagent. Concentrations of test compound sufficient to reduce CPE by 50% (EC₅₀) or decrease cell viability by 50% (CC₅₀) were interpolated using standard methods in Microsoft Excel. Toxicity was measured using CTG as above.

### Example 15.4

### Assays for EBV

Assays for EBV were performed by reported methods.³⁷ Akata cells were induced to undergo a lytic infection with 50 μg/ml of a goat anti-human IgG antibody. Experimental compounds were diluted within plates. The cells were added and incubated for 72 h. Similar plates were initiated without virus induction/addition and used for measuring cytotoxicity by the addition of CTG. For all assays, the replication of the virus was assessed by the quantification of viral DNA. For EBV, primers 5′-CCC AGG AGT CCC AGT AGT CA-3′ and 5′-CAG TTC CTC GCCTTAGGTTG-3 amplified a fragment corresponding to coordinates 96802-97234 in EBV genome (AJ507799). Compound concentrations sufficient to reduce genome copy number by 50% were calculated from experimental data as well as compound cytotoxicity.

### Example 15.5

### Assays for HIV

### Cell Culture and Treatment Strategy

Leukopaks from healthy donors were obtained from the New York Blood Center and processed by Ficoll gradient centrifugation to isolate peripheral blood mononuclear cells (PBMC). These PBMC were cultured adherently into monocyte derived macrophages (MDM), according to published protocols, on 60 mm dishes for 6 days with 10-20 ng/ml macrophage colony stimulating factor (M-CSF). MDM were pre-treated with ddhC, compound 8, compound 21, emtricitabine (Cat. No .: 10071), or DMSO vehicle at indicated concentrations for 24 hours (NIH AIDS Reagent Program, Bethesda, MD). Cells were left uninfected or infected for 24 h in the presence of one of the compounds listed above with 20 ng/mL HIVADA, a macrophage-tropic HIV strain originally derived from a person with HIV (NIH). Media was changed daily with the mentioned compounds added each time, and supernatants were collected on days 2, 3, 4, 5, 6, and 7 of infection. Each treatment condition was analyzed in duplicate plates per experiment.

At the end of each experiment, cells were imaged by light microscopy. After aspirating off media, cells were washed with PBS, and lysates were harvested in extraction buffer containing 0.1% Formic acid, 50% ACN, 40% MeOH, 10% H₂O.

### HIV Quantification

Supernatant samples were analyzed for the viral capsid protein, HIV p24, by a sensitive p24 alphaLISA according to the manufacturer's protocol (Perkin-Elmer, Waltham, MA). Treatment conditions were analyzed in duplicate plates, and each supernatant was analyzed in duplicate on a 96-well alphaLISA plate. These were then fit to a sigmoid standard curve to extrapolate HIV p24 levels in pg/mL within the assay limits of detection.

### Toxicity Assays

Drug toxicity was analyzed using an LDH assay kit according to manufacturer's instructions (Abcam #ab65393, Toronto, Canada). Briefly, supernatants from duplicate plates per treatment condition were added to a 96-well plate. Each supernatant was run in triplicate. Provided assay buffer was added to culture supernatants after mixing with a substrate solution that turns orange upon lactate exposure. After incubation for 30-45 min, colorimetric signal was quantified an OD of 450 nm. LDH was a positive control as well as cells lysed with 10% lysis buffer in media representing the “high control” to which % cytotoxicity was measured. The reference sample was either control uninfected cells treated with DMSO or negative control HIV-infected cells treated with DMSO. Culture media alone was used as a technical negative control. This background signal that was subtracted from all test wells.

### Statistical Analyses

Quantitative data were analyzed in Prism software v.9.0.1 (GraphPad Software Inc., San Diego, CA). Shapiro-Wilk tests with p=0.05 as the cutoff were used to determine normal distribution of the data. For analyses of two groups, appropriate unpaired or paired student's t tests were used for normally distributed data. Unmatched or matched-pairs signed rank tests were used when not normally distributed. When more than two groups were analyzed, one-way ANOVA was used. This was followed by a Dunnett's test or a Turkey's test to determine differences between specific groups. One-sample t tests or Wilcoxon signed rank tests were used to test fold changes for significance. The negative control was set to 1.0 for each experiment. Values of p<0.05 were considered statistically significant.

### Example 16: Anti-Viral Activity

Compounds of the invention were assayed against a wide range of viruses as detailed in Example 15 above. Activity was identified against human Cytomegalovirus (HCMV) and Epstein-Barr virus (EBV) as shown in Table 1. Activity was also identified for some compounds of the invention against EBV compared to ddhC and cidofovir as shown in Table 2. Table 3 shows that the 2′-deoxy compound 39 was also found to be active against human betaherpesvirus 6B (HHV-6B). Activity against HIV is shown in FIGS. 1-13.

TABLE 1
Antiviral activities compared to ddhC and assay standards

| | HCMV | | | EBV | | |
|---|---|---|---|---|---|---|
| | (AD169 strain in HFF cells) | | | (Akata/Akata) | | |
| | EC₅₀ | CC₅₀ | SI₅₀ | EC₅₀ | CC₅₀ | SI₅₀ |
| Compound | (μM) | (μM) | (μM) | (μM) | (μM) | (μM) |
| Control = Ganciclovir | 0.55 | >150 | >272 | — | — | — |
| Control = Cidofovir | — | — | — | 8.25 | >100 | >12 |
| 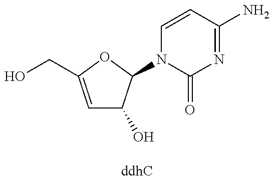 [SMILES: Nc1ccn([C@@H]2OC(CO)=C[C@H]2O)c(=O)n1] | >50 | >50 | n.d. | 38.7 | >50 | >1 |
| 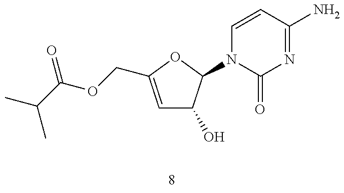 [SMILES: CC(C)C(=O)OCC1=C[C@@H](O)[C@H](n2ccc(N)nc2=O)O1] | 0.02 | >50 | >2381 | 7.6 | >50 | >7 |
| 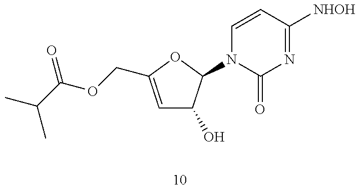 | 0.03 | >50 | >1786 | 29.9 | >50 | >2 |
| 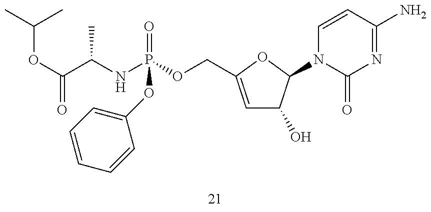 [SMILES: CC(C)OC(=O)[C@H](C)N[P@](=O)(OCC1=C[C@@H](O)[C@H](n2ccc(N)nc2=O)O1)Oc1ccccc1] | n.d. | n.d. | n.d. | 23.7 | >50 | >2 |

n.d. = not determined

TABLE 2
Epstein-Barr Virus antiviral activities compared to ddhC and
cidofovir (CDV) control
EBV (Akata/Akata) - CC₅₀ > 50 μM (primary) or >75 μM (secondary)

| | | | Normalised | | |
|---|---|---|---|---|---|
| | | CDV | value = | | |
| | Result for | control | (Compound | | |
| | compound | result | EC50/CDV | | Assay |
| Compound | (EC₅₀/UM) | (EC₅₀/UM) | control EC₅₀) | SI₅₀ | order |
| 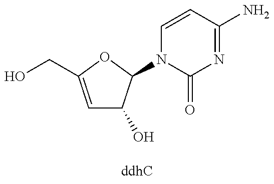 [SMILES: Nc1ccn([C@@H]2OC(CO)=C[C@H]2O)c(=O)n1] | 38.69 | 8.25 | 4.7 | >1.3 | Primary |
| 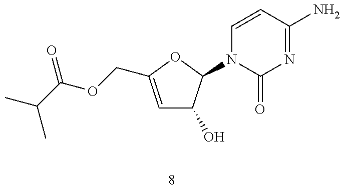 [SMILES: CC(C)C(=O)OCC1=C[C@@H](O)[C@H](n2ccc(N)nc2=O)O1] | 7.59 | 8.25 | 0.92 | >6.6 | Primary |
| 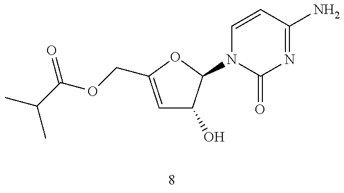 [SMILES: CC(C)C(=O)OCC1=C[C@@H](O)[C@H](n2ccc(N)nc2=O)O1] | 16.15 | 7.56 | 2.14 | >4.6 | Secondary |
| 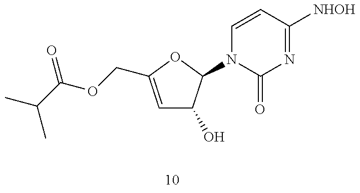 | 29.93 | 8.25 | 3.63 | >1.7 | Primary |
| 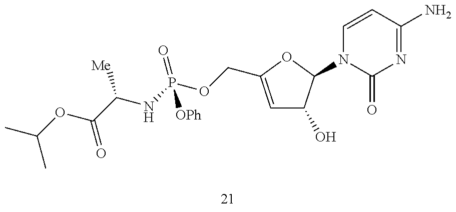 [SMILES: CC(C)OC(=O)[C@H](C)N[P@](=O)(OCC1=C[C@@H](O)[C@H](n2ccc(N)nc2=O)O1)Oc1ccccc1] | 23.75 | 8.25 | 2.88 | >2.1 | Primary |
| 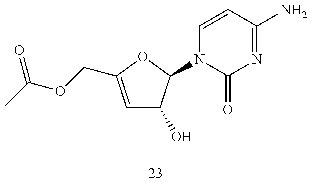 [SMILES: CC(=O)OCC1=C[C@@H](O)[C@H](n2ccc(N)nc2=O)O1] | 8.96 | 3.31 | 2.71 | >5.6 | Primary |
| 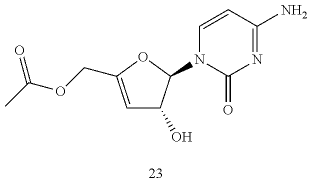 [SMILES: CC(=O)OCC1=C[C@@H](O)[C@H](n2ccc(N)nc2=O)O1] | 7.96 | 1.93 | 3.08 | >9.4 | Secondary |
| 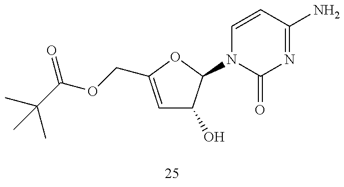 [SMILES: CC(C)(C)C(=O)OCC1=C[C@@H](O)[C@H](n2ccc(N)nc2=O)O1] | 15.87 | 5.95 | 2.67 | >4.7 | Secondary |
| 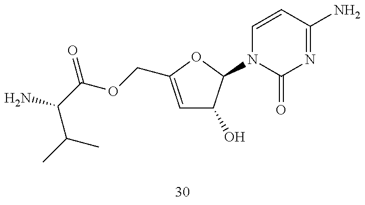 [SMILES: CC(C)[C@H](N)C(=O)OCC1=C[C@@H](O)[C@H](n2ccc(N)nc2=O)O1] | 18.47 | 5.95 | 3.1 | >4.1 | Secondary |
| 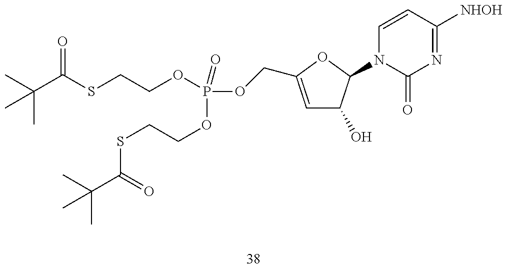 | 16.56 | 5.95 | 2.78 | >4.2 | Secondary |
| 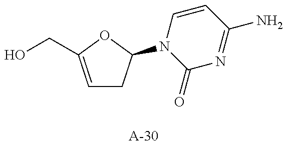 [SMILES: Nc1ccn([C@H]2CC=C(CO)O2)c(=O)n1 ; "A-30"] | 29.64 | 2.58 | 11.49 | >1.7 | Primary |
| 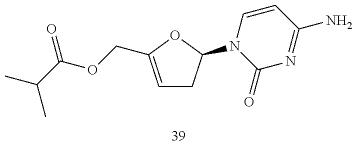 [SMILES: CC(C)C(=O)OCC1=CC[C@H](n2ccc(N)nc2=O)O1 ; "39"] | 6.31 | 2.58 | 2.44 | >7.9 | Primary |
| 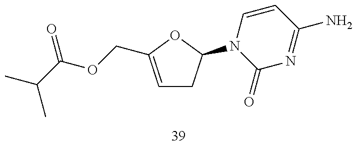 [SMILES: CC(C)C(=O)OCC1=CC[C@H](n2ccc(N)nc2=O)O1 ; "39"] | 7.09 | 1.93 | 3.67 | >10.6 | Secondary |
| 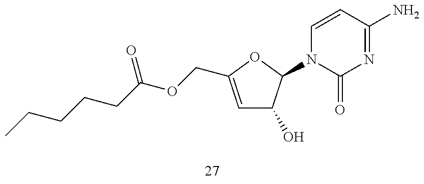 [SMILES: CCCCCC(=O)OCC1=C[C@@H](O)[C@H](n2ccc(N)nc2=O)O1] | >50 | 2.58 | 0 | 0 | Primary |
| 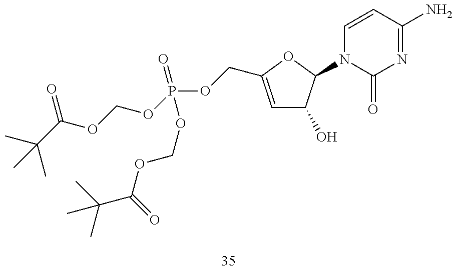 [SMILES: CC(C)(C)C(=O)OCOP(=O)(OCOC(=O)C(C)(C)C)OCC1=C[C@@H](O)[C@H](n2ccc(N)nc2=O)O1] | >50 | 2.58 | 0 | 0 | Primary |
| 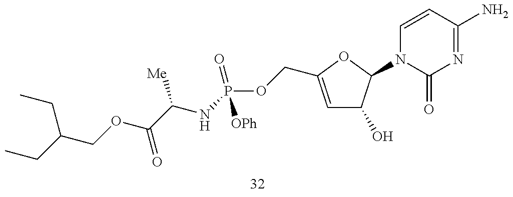 [SMILES: CCC(CC)COC(=O)[C@H](C)N[P@](=O)(OCC1=C[C@@H](O)[C@H](n2ccc(N)nc2=O)O1)Oc1ccccc1] | >50 | 1.15 | 0 | 0 | Primary |

TABLE 3
Activity of 2'-deoxy compound 39 against human
betaherpesvirus 6B (HHV-6B)
HHV-6B (Z29; MOLT-3) - CC₅₀ > 50 μM (primary) or >75 μM (secondary)

| | | | Normalised | | |
|---|---|---|---|---|---|
| | | CDV | value = | | |
| | Result for | control | (Compound | | |
| | compound | result | EC₅₀/CDV | | Assay |
| Compound | (EC₅₀/UM) | (EC₅₀/UM) | control EC₅₀) | SI₅₀ | order |
| 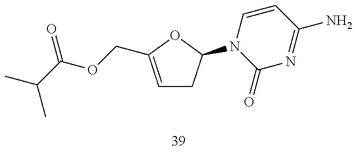 [SMILES: CC(C)C(=O)OCC1=CC[C@H](n2ccc(N)nc2=O)O1] | 5.54 | 3.42 | 1.62 | >9 | Primary |
| 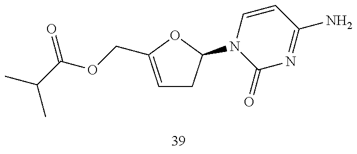 [SMILES: CC(C)C(=O)OCC1=CC[C@H](n2ccc(N)nc2=O)O1] | 16.67 | 4.26 | 3.91 | >4.5 | Secondary |

Although the invention has been described by way of example, it should be appreciated that variations and modifications may be made without departing from the scope of the invention as defined in the claims. Furthermore, where known equivalents exist to specific features, such equivalents are incorporated as if specifically referred in this specification.

### REFERENCES

1. Crumpacker, C. S., Cytomegalovirus (CMV). In Mandell, Douglas, and Bennett's Principles and Practice of Infectious Disease, 8th ed ed.; Bennett, J. E.; Dolin, R.; Blaser, M. J., Eds. Elsevier Saunders: Philadelphia, PA, 2015.
2. Öberg, B., Antiviral effects of phosphonoformate (PFA, foscarnet sodium). Pharmacology & therapeutics 1989, 40 (2), 213-285.
3. Biron, K. K.; Stanat, S. C.; Sorrell, J. B.; Fyfe, J. A.; Keller, P. M.; Lambe, C. U.; Nelson, D. J., Metabolic activation of the nucleoside analog 9-[(2-hydroxy-1-(hydroxymethyl) ethoxy] methyl) guanine in human diploid fibroblasts infected with human cytomegalovirus. Proceedings of the National Academy of Sciences 1985, 82 (8), 2473-2477.
4. Xiong, X.; Smith, J. J.; Chen, M. S., Effect of incorporation of cidofovir into DNA by human cytomegalovirus DNA polymerase on DNA elongation. Antimicrobial agents and chemotherapy 1997, 41 (3), 594-599.
5. Chou, S., Cytomegalovirus UL97 mutations in the era of ganciclovir and maribavir. Reviews in medical virology 2008, 18 (4), 233-246.
6. Lacy, S. A.; Hitchcock, M. J.; Lee, W. A.; Tellier, P.; Cundy, K. C., Effect of oral probenecid coadministration on the chronic toxicity and pharmacokinetics of intravenous cidofovir in cynomolgus monkeys. Toxicological Sciences 1998, 44 (2), 97-106.
7. Marty, F. M.; Winston, D. J.; Chemaly, R. F.; Mullane, K. M.; Shore, T. B.; Papanicolaou, G. A.; Chittick, G.; Brundage, T. M.; Wilson, C.; Morrison, M. E., A randomized, double-blind, placebo-controlled phase 3 trial of oral brincidofovir for cytomegalovirus prophylaxis in allogeneic hematopoietic cell transplantation. Biology of Blood and Marrow Transplantation 2019, 25 (2), 369-381.
8. Chou, S., Rapid in vitro evolution of human cytomegalovirus UL56 mutations that confer letermovir resistance. Antimicrobial agents and chemotherapy 2015, 59 (10), 6588.
9. Acosta, E.; Bowlin, T.; Brooks, J.; Chiang, L.; Hussein, I.; Kimberlin, D.; Kauvar, L. M.; Leavitt, R.; Prichard, M.; Whitley, R., Advances in the development of therapeutics for cytomegalovirus infections. The Journal of infectious diseases 2020, 221 (Supplement_1), S32-S44.
10. Jangra, S.; Yuen, K .-S.; Botelho, M. G.; Jin, D .-Y., Epstein-Barr virus and innate immunity: Friends or foes? Microorganisms 2019, 7 (6), 183.
11. Andrei, G.; Trompet, E.; Snoeck, R., Novel therapeutics for Epstein-Barr virus. Molecules 2019, 24 (5), 997.
12. Hocker, B.; Böhm, S.; Fickenscher, H.; Küsters, U.; Schnitzler, P.; Pohl, M.; John, U.; Kemper, M. J.; Fehrenbach, H.; Wigger, M., (Val-) Ganciclovir prophylaxis reduces Epstein-Barr virus primary infection in pediatric renal transplantation. Transplant International 2012, 25 (7), 723-731.
13. Yager, J. E.; Magaret, A. S.; Kuntz, S. R.; Selke, S.; Huang, M .-L.; Corey, L.; Casper, C.; Wald, A., Valganciclovir for the suppression of Epstein-Barr virus replication. The Journal of infectious diseases 2017, 216 (2), 198-202.
14. Funch, D. P.; Walker, A. M.; Schneider, G.; Ziyadeh, N. J.; Pescovitz, M. D., Ganciclovir and acyclovir reduce the risk of post-transplant lymphoproliferative disorder in renal transplant recipients. American Journal of Transplantation 2005, 5 (12), 2894-2900.
15. Snoeck, R.; De Clercq, E., Role of cidofovir in the treatment of DNA virus infections, other than CMV infections, in immunocompromised patients. Current opinion in investigational drugs (London, Englandl 2000) 2002, 3 (11), 1561-1566.
16. Coen, N.; Duraffour, S.; Haraguchi, K.; Balzarini, J.; van den Oord, J. J.; Snoeck, R.; Andrei, G., Antiherpesvirus activities of two novel 4′-thiothymidine derivatives, KAY-2-41 and KAH-39-149, are dependent on viral and cellular thymidine kinases. Antimicrobial agents and chemotherapy 2014, 58 (8), 4328-4340.
17. Chin, K .-C.; Cresswell, P., Viperin (cig5), an IFN-inducible antiviral protein directly induced by human cytomegalovirus. Proceedings of the National Academy of Sciences 2001, 98 (26), 15125-15130.
18. Helbig, K. J.; Beard, M. R., The role of viperin in the innate antiviral response. Journal of molecular biology 2014, 426 (6), 1210-9.
19. Gizzi, A. S.; Grove, T. L.; Arnold, J. J.; Jose, J.; Jangra, R. K.; Garforth, S. J.; Du, Q.; Cahill, S. M.; Dulyaninova, N. G.; Love, J. D.; Chandran, K.; Bresnick, A. R.; Cameron, C. E.; Almo, S. C., A naturally occurring antiviral ribonucleotide encoded by the human genome. Nature 2018, 558 (7711), 610-614.
20. Gizzi, A. S.; Grove, T. L.; Arnold, J. J.; Jose, J.; Jangra, R. K.; Garforth, S. J.; Du, Q.; Cahill, S. M.; Dulyaninova, N. G.; Love, J. D.; Chandran, K.; Bresnick, A. R.; Cameron, C. E.; Almo, S. C., Author Correction: A naturally occurring antiviral ribonucleotide encoded by the human genome. Nature 2020, 583 (7814), E15-E15.
21. Ghosh, S.; Marsh, E. N. G., Viperin: An ancient radical SAM enzyme finds its place in modern cellular metabolism and innate immunity. The Journal of biological chemistry 2020, 295 (33), 11513-11528.
22. Rivera-Serrano, E. E.; Gizzi, A. S.; Arnold, J. J.; Grove, T. L.; Almo, S. C.; Cameron, C. E., Viperin Reveals Its True Function. Annual review of virology 2020, 7 (1), 421-446.
23. Kumamoto, H.; Shindoh, S.; Tanaka, H.; Itoh, Y.; Haraguchi, K.; Gen, E.; Kittaka,

A.; Miyasaka, T.; Kondo, M.; Nakamura, K. T., An Intramolecular Anionic Migration of a Stannyl Group from the 6-Position of 1-(2-Deoxy-d-erythro-pent-1-enofuranosyl) uracil to the 2′-Position: Synthesis of 2′-Substituted 1′, 2′-Unsaturated Uridines. Tetrahedron 2000, 56 (30), 5363-5371.
24. Navacchia, M. L.; Montevecchi, P. C., Sulfanyl radical promoted C4′-C5′ bond scission of 5′-oxo-3′, 4′-didehydro-2′, 3′-dideoxynucleosides. Org Biomol Chem 2006, 4 (20), 3754-3756.
25. Brown, J. A.; Suo, Z., Unlocking the sugar “steric gate” of DNA polymerases. Biochemistry 2011, 50 (7), 1135-1142.
26. Guo, C.; Hainan, W. 3′-Deoxy-3′,4′-didehydrogenized Nucleoside Compound and Application Thereof. CN 108640959 B, 2018/07/06, 2018.
27. Almo, S.; Grove, T.; Gizzi, A.; Cameron, C.; Arnold, J. Broad Spectrum Viral Inhibitor. WO 2019/040418 A1, 2018/08/21, 2019.
28. Passow, K.T; et al., J. Med. Chem. 2021, 64, 20, 15429-15439.
29. WO 2020/202142 30. Schalke, P. M.; Hall, C. D., Mechanism of the reaction of cytosine with hydroxylamines. Journal of the Chemical Society, Chemical Communications 1976, (11), 391-392.
31. Miller, P. S. Formation Of Oligonucleotide Triplexes With Selectively Modified Cytosines. US 5407801 A, 1993/08/03, 1995.
32. Zhang, J.; Chang, H. M.; Kane, R. R., Reductive monoalkylation of aromatic amines via amidine intermediates. Synlett 2001, (5), 643-645.
33. Adamska, E.; Barciszewski, J.; Markiewicz, W. T., Convenient and Efficient Syntheses of N 6- and N 4- Substituted Adenines and Cytosines and their 2′-Deoxyribosides. Nucleosides, Nucleotides & Nucleic Acids 2012, 31 (12), 861-871.
34. Petrová, M., et al., Tetrahedron Lett. 2010, 51, 6874-6876.
35. Ogilvie, K. K.; Beaucage, S. L.; Schifman, A. L.; Theriault, N. Y.; Sadana, K. L., The synthesis of oligoribonucleotides. II. The use of silyl protecting groups in nucleoside and nucleotide chemistry. VII. Canadian Journal of Chemistry 1978, 56 (21), 2768-2780.
36. Ross, B. S.; Ganapati Reddy, P.; Zhang, H .-R.; Rachakonda, S.; Sofia, M. J., Synthesis of Diastereomerically Pure Nucleotide Phosphoramidates. The Journal of Organic Chemistry 2011, 76 (20), 8311-8319.
37. Hartline, C. B.; Keith, K. A.; Eagar, J.; Harden, E. A.; Bowlin, T. L.; Prichard, M. N., A standardized approach to the evaluation of antivirals against DNA viruses: Orthopox-, adeno-, and herpesviruses. Antiviral Research 2018, 159, 104-112.
38. Prichard, M. N.; Keith, K. A.; Quenelle, D. C.; Kern, E. R., Activity and mechanism of action of N-methanocarbathymidine against herpesvirus and orthopoxvirus infections. Antimicrobial agents and chemotherapy 2006, 50 (4), 1336-1341.

1. A compound of the Formula (I):
wherein: 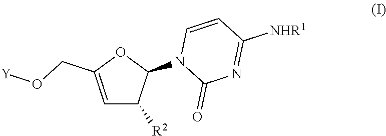
R¹ is H, OH, a C₁₋₆ alkyl group or a cyclopropyl group;
R² is H or OH;
Y is R³, R⁴CO, PO(OR⁵)₂, PO(OR⁶)(OH), or PO(OR⁷)(X);
R³ is a pivaloyl-, isobutyroyl- or isopropyloxycarbonyloxy-(C1-3 alkyl)methyl group;
R⁴ is C₁₋₂₀ alkyl, C₆₋₁₂ aryl, C₁₋₆ alkyl-C6-aryl, 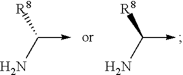
R⁵ is a S-pivaloyl-2-thioethyl, pivaloyloxymethyl or isopropyloxycarbonyloxymethyl group;
R⁶ is a 3-(C₁₂₋₁₈ alkoxy)propyl group;
R⁷ is C₆₋₁₂ aryl;
X is 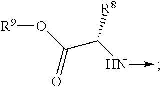
R⁸ is the side group of a natural amino acid; and
R⁹ is C₁₋₅ alkyl or C₆₋₁₂ aryl;
or a pharmaceutically acceptable salt thereof.

2. A compound as claimed in claim 1, wherein R¹ is H.

3. A compound as claimed in claim 2, wherein R¹ is OH.

4. A compound as claimed in claim 1, wherein R² is OH.

5. A compound as claimed in claim 1, wherein R¹ is H and R² is OH.

6. A compound as claimed in claim 1, wherein Y is R^{3.}

7. A compound as claimed in claim 1, wherein Y is R⁴CO or PO(OR⁵)₂.

8. A compound as claimed in claim 6, wherein Y contains an isopropyl group or a t-butyl group.

9. A compound as claimed in claim 1, wherein Y is PO(OR⁵)₂, PO(OR⁶)(OH), or PO(OR⁷)(X).

10. A compound as claimed in claim 1, wherein R¹ is OH, a C₁₋₆ alkyl group or a cyclopropyl group, Y is R³, R⁴CO, PO(OR⁶)(OH), or PO(OR⁷)(X), and R⁵ is a pivaloyloxymethyl or isopropyloxycarbonyloxymethyl group

11. A compound as claimed in claim 1 selected from the group comprising:

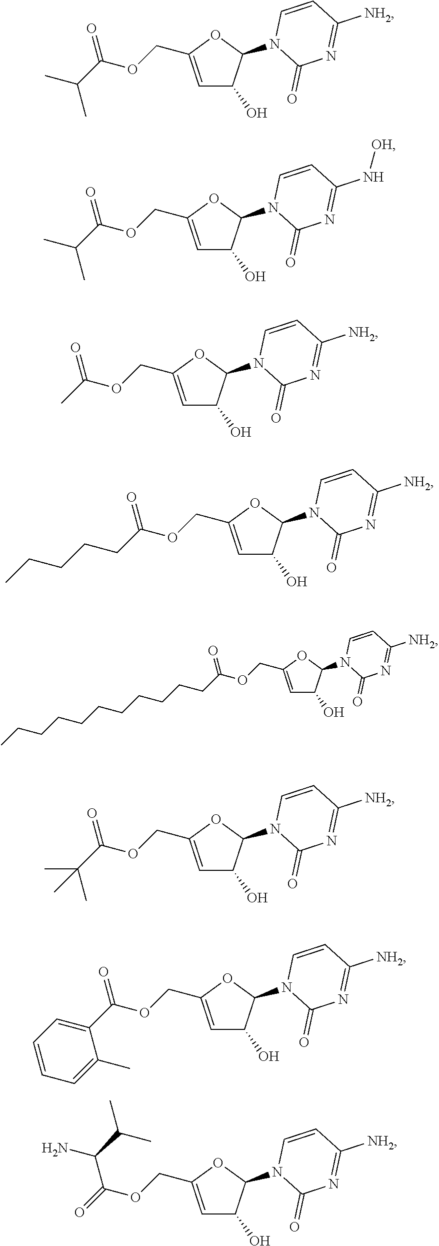

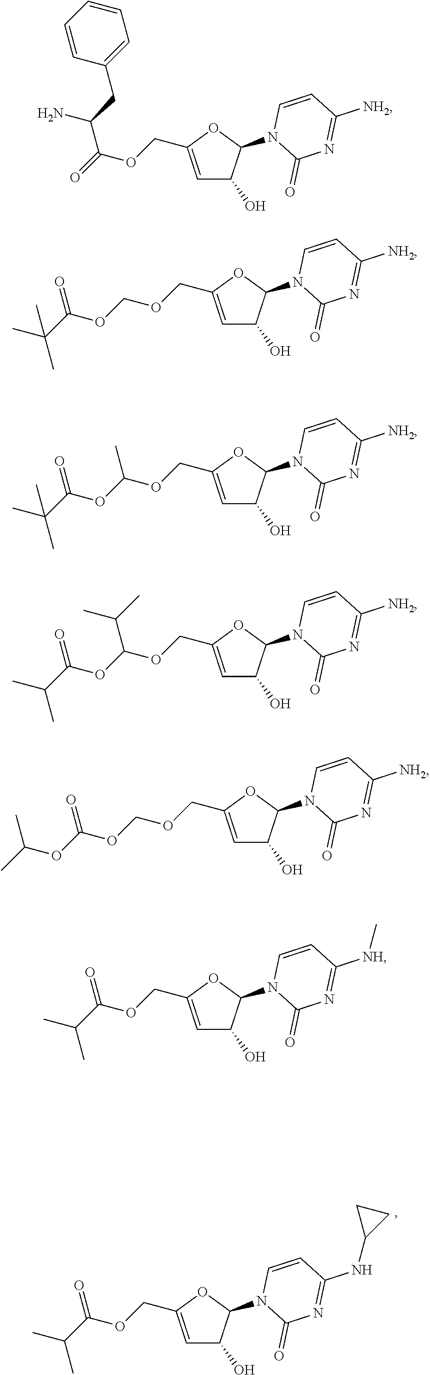

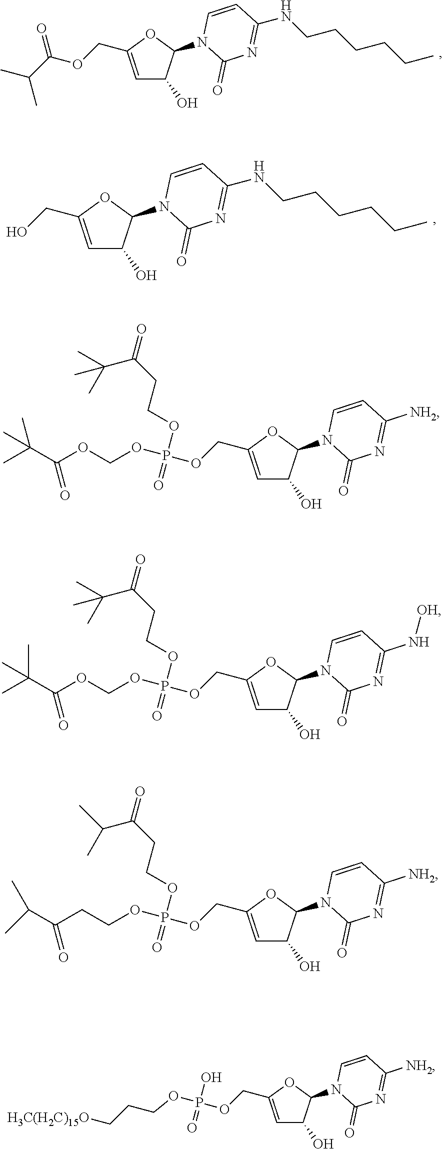

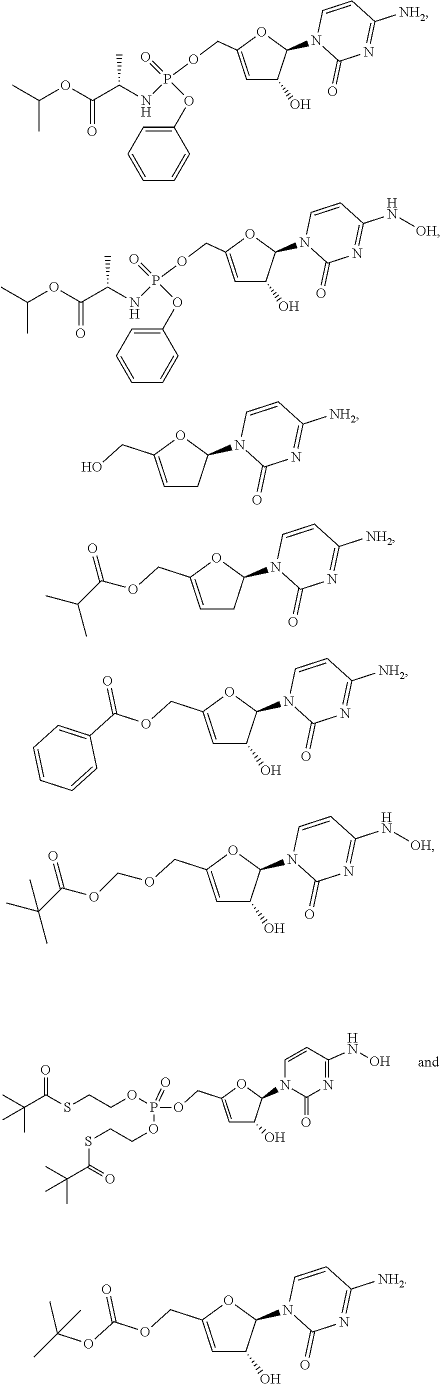

12. A pharmaceutical composition comprising a compound as claimed any claim 1 and a pharmaceutically acceptable carrier.

13. A method of treating or preventing an infection caused by Human Cytomegalovirus (HCMV), Epstein-Barr virus (EBV), or Human Immunodeficiency Virus (HIV) comprising administering to a human in need an effective amount of a compound of claim 1
